(19) United States
(12) Patent Application Publication
(10) Pub. No.: US 2024/0172949 A1
(43) Pub. Date: May 30, 2024

(54) SYSTEMS AND METHODS FOR MEASURING INTRACRANIAL PRESSURE

(71) Applicants: Nelson L. JUMBE, Mountain View, CA (US); Andreas SCHUH, Millbrae, CA (US); Michael MORIMOTO, Dublin, CA (US); Peter REXELIUS, San Francisco, CA (US); Steve KRAWCZYK, Palo Alto, CA (US); Andrew URAZAKI, Las Vegas, NV (US)

(72) Inventors: Nelson L. JUMBE, Mountain View, CA (US); Andreas SCHUH, Millbrae, CA (US); Michael MORIMOTO, Dublin, CA (US); Peter REXELIUS, San Francisco, CA (US); Steve KRAWCZYK, Palo Alto, CA (US); Andrew URAZAKI, Las Vegas, NV (US)

(21) Appl. No.: 18/552,024

(22) PCT Filed: Mar. 24, 2022
(86) PCT No.: PCT/US2022/021797

§ 371 Date: Sep. 22, 2023

Related U.S. Application Data
(60) Provisional application No. 63/165,610, filed on Mar. 24, 2021.
(60) Provisional application No. 63/165,618, filed on Mar. 24, 2021.

(51) Int. Cl. A61B 5/03; A61B 5/00; A61B 5/053; A61B 7/00

(52) U.S. Cl. CPC A61B 5/031; A61B 5/053; A61B 5/6803; A61B 7/001; A61B 2562/0204

(57) ABSTRACT

There is disclosed a system for monitoring, non-invasively, intracranial pressure of a subject. The system includes a vibroacoustic sensor and an electric potential sensor. The vibroacoustic sensor is configured to detect vibroacoustic signals associated with intracranial pressure of the subject, within a bandwidth ranging from about 0.01 Hz to about 20 kHz. The electric potential sensor is configured to detect electric potential signals reflective of baseline time-based events in the subject for identifying baseline time-based intracranial pressure changes from the detected vibroacoustic signals. The vibroacoustic sensor is housed in a wearable device. The wearable device is configured to be non-invasively coupled to the subject's head.

### CROSS-REFERENCE TO RELATED APPLICATION

This application claims the benefit of U.S. Provisional Application No. 63/165,618, filed on Mar. 24, 2021, and U.S. Provisional Application No. 63/165,610, filed on Mar. 24, 2021, each of which is incorporated by reference herein in its entirety.

### BACKGROUND

The natural resonance of a subject's skull—the unique frequency at which the bones in a person's skull vibrates affects how subject hears and experiences sound. Inaudible low frequency vibrations (<20 Hz, infrasound) are often not considered to be sound at all. Humans can hear very low-frequency sounds at levels above 88-100 dB down to a few cycles per second, but cannot get any tonal information out of the sounds below about 20 Hz—it mostly just feels like beating pressure waves. And like any other sound, if presented at levels above 140 dB, it is going to cause pain. But the primary effects of infrasound are not on a subject's ears but on the rest of their body.

The low frequency of infrasonic sound and its corresponding long wavelength makes it much more capable of bending around or penetrating a subject's body, creating an oscillating pressure system. Depending on the frequency, different parts of the body will resonate, which can have very unusual non-auditory effects. For example, one of the effects that occurs at relatively safe sound levels (<100 dB) occurs at 19 Hz. If a subject is positioned in front of a high-quality subwoofer playing a 19 Hz sound, their eyes will twitch. If the volume is turned up approaching 110 dB, the subject may even start seeing colored lights at the periphery of their vision or ghostly gray regions in the center. This is because 19 Hz is the resonant frequency of the human eyeball. The low-frequency pulsations start distorting the eyeball's shape and pushing on the retina, activating the rods and cones by pressure rather than light.

Almost any part of the human body, based on its volume and makeup, will vibrate at specific frequencies with enough power. Human eyeballs are fluid-filled ovoids, lungs are gas-filled membranes, and the human abdomen contains a variety of liquid-, solid-, and gas-filled pockets. All these structures have limits to how much they can stretch when subjected to force, so if enough power is provided behind a vibration, they will stretch and shrink in time with the low-frequency vibrations of the air molecules around them.

Because humans do not hear infrasonic frequencies very well, subjects are often unaware of exactly how loud sounds are. At 130 dB, the inner ear will start undergoing direct pressure distortions unrelated to normal hearing, which can affect the ability to understand speech. At about 150 dB, people may start complaining about nausea and whole-body vibrations, usually in the chest and abdomen. By the time 166 dB is reached, people may start noticing problems breathing, as the low-frequency pulses start impacting the lungs, reaching a critical point at about 177 dB, when infrasound from 0.5 to 8 Hz can actually drive sonically induced artificial respiration at an abnormal rhythm. In addition, vibrations through a substrate such as the ground can be passed throughout a subject's body via the skeleton, which in turn can cause the subject's whole body to vibrate at 4-8 Hz vertically and 1-2 Hz side to side. The effects of this type of whole-body vibration can cause many problems, ranging from bone and joint damage with short exposure to nausea and visual damage with chronic exposure. The commonality of infrasonic vibration, especially in the realm of heavy equipment operation, has led federal and international health and safety organizations to create guidelines to limit people's exposure to this type of infrasonic stimulus.

The ability to understand spoken language is a defining human capacity. But despite decades of research, there is still no well-specified account of how sound entering the ear is neurally interpreted as a sequence of meaningful words. A fundamental concern in the human sciences is to relate the study of the neurobiological systems supporting complex human cognitive functions to the development of computational systems capable of emulating or even surpassing these capacities. Spoken language comprehension is a salient domain that depends on the capacity to recognize fluent speech, decoding word identities and their meanings from a stream of rapidly varying auditory input.

In humans, the language vocalization process is learned subconsciously and very quickly by newborns and depends on a highly dynamic set of electrophysiological processes in speech- and language-related brain areas. These processes extract salient phonetic cues which are mapped onto abstract word identities as a basis for linguistic interpretation. But the exact nature of these processes, their computational content, and the organization of the neural systems that support them, are far from being understood. The rapid, parallel development of Automatic Speech Recognition (ASR) systems, with near-human levels of performance, means that computationally specific solutions to the speech recognition problem are now emerging, built primarily for the goal of optimizing accuracy, with little reference to potential neurobiological constraints and/or physiobiological underpinnings.

With advancements in human-computer interfaces, communication with machines is more intuitive than ever. These natural user interfaces, however, rely on a person's ability to control voluntary movements. These user interfaces do not provide a solution for subjects who are immobilized or situationally impaired and cannot type, gesticulate, tap, or speak? A non-speech or movement based solution would be applicable to many uses, such as, but not limited to facilitating communications in noisy environments, detection of deceptive intent, clandestine operations, brain-machine interfaces and psychotherapy.

Existing technology requires the use of invasive sensors in order to achieve useful sensitivity and specificity, such as implantable electrodes. Moreover, because the invasive neural sensors harvest a superposition of electrical fields representing a myriad of neural functions, it is difficult or impossible to disambiguate the signals into intelligible data that accurately represents the desired psychological actions and states.

It is therefore desired to provide methods and systems which overcomes the above-noted shortcomings.

### SUMMARY

Broadly, there are provided devices, systems and methods of the present technology, configured to detect data associated with a brain and/or a skull of a subject. The data may include passive vibroacoustic data, active vibroacoustic data, pressure fluctuations simultaneous with/without electric potential data or electroencephalogram (EEG) data. Data relating to the subject's heartbeat, breath and/or blood flow may also be simultaneously detected. Data relating to an environment of the subject may also be simultaneously detected.

According to certain embodiments, the detected data can be processed to: (1) determine and/or monitor an intracranial pressure of the subject, (2) determine and/or monitor an intent of the subject which may be a thought, a command, a word, an image, etc, and/or (3) determine a state or condition of the subject.

The present technology may further comprise causing the control of a machine, a software and/or other electrical systems using one or more of the determined intracranial pressure, intent, registration of perception and state or condition. The determined intracranial pressure, intent and state or condition may permit providing a treatment to the subject to maintain or change the determined state or condition.

Advantageously, according to embodiments of the present technology, the devices and systems of the present technology include one or more sensors which are non-invasive. Such sensors may be embodied in one or more wearable devices. The sensors can pick up non-audible frequencies.

From one aspect, there is provided a system for monitoring, non-invasively, intracranial pressure of a subject, the system comprising: a vibroacoustic sensor configured to detect vibroacoustic signals associated with intracranial pressure of the subject, the vibroacoustic signals being within a bandwidth ranging from about 0.01 Hz to about 20 kHz (or in inaudible range); and an electric potential sensor configured to detect electric potential signals reflective of baseline time-based events in the subject for identifying baseline time-based intracranial pressure changes from the detected vibroacoustic signals, wherein the at least one vibroacoustic sensor is housed in a wearable device which is configured to be non-invasively coupled to a head of the subject.

In certain embodiments, the vibroacoustic sensor and the electric potential sensor are configured to obtain the vibroacoustic signals and the electric potential signals in a time-locked manner.

In certain embodiments, the baseline time-based events of the subject comprise heartbeats and/or breaths. Such time-based events cause pulsatile intracranial pressure changes which are termed the “baseline time-based intracranial pressure changes”. As the vibroacoustic and the electric potential signals are time-locked, the electric potential signals can therefore be used to identify the baseline time-based intracranial pressure changes from the vibroacoustic data.

This can then enable the disambiguation of the baseline time-based intracranial pressure changes from the vibroacoustic data, to determine, if any intracranial pressure changes based on other events. Any changes of the intracranial pressure, additional to the pulsatile intracranial pressure from the time-based events of the subject, are referred to herein as an “intracranial pressure changes”. The intracranial pressure changes may be defined for example by a magnitude, a frequency pattern and/or an aperiodic pattern.

In certain embodiments, the intracranial pressure changes may be compared to a threshold magnitude, a frequency and/or an aperiodic pattern of intracranial pressure changes to determine an occurrence of an intracranial pressure event. Such intracranial pressure events may thus be detected and/or monitored using embodiments of the present technology.

Intracranial pressure events of the subject may be related to conditions associated with the subject or may be contextual related.

For intracranial pressure events related to conditions, the intracranial pressure event can be compared to biomarkers of various conditions to identify if the subject has an onset of a given condition, a precursor to a given condition, or an increase/decrease in the condition. The condition can be an event such as a fall or an impact of the subject. The condition can be the presence or absence of a disease. The condition can be a progression of a disease state such as a tumor, a hemorrhage, etc).

For contextual-related intracranial pressure changes (such as atmospheric conditions, external events, etc) those time-based changes can then be compared to biomarkers.

In certain embodiments, the system includes a plurality of the vibroacoustic sensors configured to be positioned at different locations on the head of the subject.

The vibroacoustic sensor and/or the plurality of vibroacoustic sensors may be positioned at a base of the skull, such as at the cisterna magna. Another vibroacoustic sensor may be positioned proximate a temple of the subject.

In certain embodiments, the vibroacoustic sensor comprises at least one voice coil sensor.

In certain embodiments, the electric potential sensor is housed in the wearable device.

The electric potential sensor may be co-located with the vibroacoustic sensor. Alternatively, the electric potential sensor may be positioned on the subject but not in the wearable device. The electric potential sensor may be housed in another wearable device, such as a patch.

Alternatively, the electric potential sensor may be positioned remote from the subject and configured to detect the electric potential signals remotely.

In certain embodiments, the wearable device comprises an earpiece positionable in or over the ear of the subject, and the vibroacoustic sensor comprises a voice coil sensor in the earpiece.

In certain embodiments, the system further comprises a speaker configured to emit a signal, the speaker housed in the earpiece and separated from the voice coil sensor by a dampener.

In certain embodiments, the dampener may enable control of interaction between the sensor and the speaker. When the sensor comprises a voice coil including a sensing magnet, and the speaker includes a voice coil with a speaker magnet, an interaction may be desired between the sensing magnet and the speaker magnet, for example a harmonic relationship between active and passive sensing e.g. relaxing soundscapes, audio stimuli.

In certain embodiments, the signal is a predetermined vibroacoustic signal pattern retrieved from a sound library. The predetermined vibroacoustic signal pattern may be a sweep-frequency signal pattern.

In certain embodiments, the system is configured such that one or both of the vibroacoustic and electric potential sensors measure respective one or both of the vibroacoustic and electric potential signals of the subject responsive to the signal being provided to the subject.

In certain embodiments, the wearable device comprises two earpieces, each earpiece positionable in or over a respective ear of the subject, and the vibroacoustic sensor comprises at least one voice coil sensor in each ear piece, whereby the vibroacoustic signals detected in each earpiece can identify differences associated with left and right brain hemispheres of the subject. In certain embodiments, there may be provided at least one electric potential sensor in each earpiece. In certain embodiments, a speaker of one earpiece is configured to emit a signal and the vibroacoustic or electric potential sensor of the other earpiece is configured to detect signals from the subject responsive to the emitted signal. This could tap into left and right hemisphere responses of the subject's brain.

In certain embodiments, the wearable device comprises two earpieces, each earpiece positionable in or over a respective ear of the subject, and the vibroacoustic sensor comprises at least one voice coil sensor housed in one ear piece, and a speaker configured to emit a signal housed in the other ear piece.

The emitted signal may be audible to the subject or within an inaudible frequency range for the subject.

The configuration of having two earpieces allows for the benefits of two simultaneous sampling of signals which can be used for noise averaging, and planar locational sensing of specific tissues of interest. In embodiments containing three or more sensors, the noise averaging can be further enhanced and 3D locational sensing of specific tissues of interest is achievable.)

In certain embodiments, the signal is a predetermined vibroacoustic signal pattern retrieved from a sound library, the speaker being configured to emit the predetermined signal pattern.

In certain embodiments, the system is configured such that one or both of the vibroacoustic and electric potential sensors measure one or both of the respective vibroacoustic and electric potential signals responsive to the signal being provided to the subject.

In certain embodiments, the wearable device comprises a patch configured to be non-invasively coupled to a skin of the subject.

In certain embodiments, the system further comprises a patch configured to be non-invasively coupled to a skin of the subject, the patch including the electric potential sensor or another electric potential sensor.

In certain embodiments, the system further comprises: a patch configured to be non-invasively coupled to a skin of the subject, the patch including another vibroacoustic sensor.

In certain embodiments, the system further comprises a patch configured to be non-invasively coupled to a skin of the subject, the patch including another vibroacoustic sensor and the electric potential sensor and/or another electric potential sensor. The patch may be configured to be attached to the skin proximate a carotid artery of the subject.

In certain embodiments, the system further comprises a remote device for providing a signal to the subject, the signal being one or more of a vibroacoustic signal, a sound signal, a haptic signal, and a visual signal.

In certain embodiments, the signal is a predetermined vibroacoustic signal pattern retrieved from a sound library, the remote device being configured to emit the predetermined vibroacoustic signal pattern.

In certain embodiments, the system is configured such that one or both of the vibroacoustic and electric potential sensors measure one or both of the respective vibroacoustic and electric potential signals from the subject responsive to the signal being provided to the subject by the remote device.

In certain embodiments, the remote device includes another electric potential sensor for remotely detecting an electric potential associated with the subject.

In certain embodiments, the system further comprises one or more sensors selected from: an infrared thermographic camera for detecting temperature changes associated with nasal and/or oral airflow (e.g. breath); a machine vision camera for detecting one or more of: facial movement of the subject, chest movement of the subject, eye tracking of the subject and iris color scanning of the subject; and a sensor for detecting volatile organic compounds emanating from the subject.

In certain embodiments, the system further comprises: an augmented/virtual reality head-piece wearable by the subject.

In certain embodiments, the vibroacoustic sensor has a vibroacoustic sensor sampling rate for capturing the vibroacoustic signals and the electric potential sensor has an electric potential sensor sampling rate for capturing the electric potential signals, each of the vibroacoustic sensor sampling rate and the electric potential sensor sampling rate being determined to optimize the battery life of the respective vibroacoustic sensor and the electric potential sensor.

In certain embodiments, the vibroacoustic sensor has a vibroacoustic sensor sampling rate for capturing the vibroacoustic signals and the electric potential sensor has an electric potential sensor sampling rate for capturing the electric potential signals, and the respective sampling rates of the vibroacoustic sensor and the electric potential sensor can be switched between a relatively high sampling rate and a relatively low sampling rate to optimize resolution and optimize battery life respectively.

The higher sampling rate may allow for higher sensitivity and lower specificity for high severity of a diagnosis, thereby allowing detection with less false-negatives by the machine learning algorithm.

The lower sampling rate may allow for greater differentiation of longitudinal therapeutic effect as it can be tuned for lower sensitivity and higher specificity.

From another aspect, there is provided a method for monitoring, non-invasively, intracranial pressure of a subject, the method executable by a processor of an electronic device, the method comprising: obtaining, from a vibroacoustic sensor, vibroacoustic data within a bandwidth ranging from about 0.01 Hz to about 20 kHz, the vibroacoustic data associated with intracranial pressure of the subject over at least one heart cycle of the subject; obtaining, an electric potential sensor, electric potential data associated with the subject over the at least one heart cycle of the subject; wherein the vibroacoustic data is used to determine an intracranial pressure of the subject, and the electric potential data is used to determine baseline time-based events in the subject for identifying baseline time-based intracranial pressure changes from the detected vibroacoustic signals.

In certain embodiments, the method further comprises: storing, in a memory of the electronic device, the obtained vibroacoustic data and the electric potential data.

In certain embodiments, the method further comprises: sending, by a communication module of the electronic device, the obtained vibroacoustic data and the electric potential data to a processor of a computer system.

In certain embodiments, the method further comprises: obtaining the vibroacoustic data at a vibroacoustic data sampling rate, the vibroacoustic sampling rate having been determined based on optimizing a battery life of the vibroacoustic sensor; and obtaining the electric potential data at an electric potential data sampling rate, the electric potential rate having been determined based on optimizing a battery life of the electric potential sensor.

In certain embodiments, the method further comprises obtaining the vibroacoustic data at a vibroacoustic data sampling rate; obtaining the electric potential data at an electric potential data sampling rate, each of the vibroacoustic sensor sampling rate and the electric potential sensor sampling rates being optimized for time-locking of the captured signals.

In certain embodiments, the method further comprises obtaining the vibroacoustic data at a vibroacoustic data sampling rate, obtaining the electric potential data at an electric potential data sampling rate, switching the respective sampling rates of the vibroacoustic sensor and the electric potential sensor between a relatively high sampling rate and a relatively low sampling rate to optimize data resolution and optimize battery life, respectively.

In certain embodiments, the intracranial pressure is determined by applying a trained machine learning algorithm to the received vibroacoustic data and the electric potential data.

From a further aspect, there is provided a method for monitoring an intracranial pressure of a subject, the method executable by a processor of a computer system, the method comprising: receiving vibroacoustic data from a vibroacoustic sensor configured to non-invasively detect vibroacoustic signals associated with the subject within a bandwidth ranging from about 0.01 Hz to about 20 kHz, the vibroacoustic data having been collected from the subject over at least one heart cycle of the subject; receiving electric potential data from an electric potential sensor, the electric potential data having been collected non-invasively from the subject over the at least one heart cycle of the subject; determining, using the received vibroacoustic data, intracranial pressure of the subject; and determining, using the received electric potential data, baseline time-based events in the subject, and identifying baseline time-based intracranial pressure changes from the detected vibroacoustic signals.

In certain embodiments, the method further comprises identifying, from the determined intracranial pressure any intracranial pressure changes relative to the baseline time-based intracranial pressure changes.

In certain embodiments, the method further comprises comparing the intracranial pressure changes to a biomarker of a condition to determine a presence of the condition in the subject.

In certain embodiments, the method further comprises quantifying a magnitude, a frequency pattern and/or an aperiodic pattern.

of the intracranial pressure changes to determine a condition of the subject.

In certain embodiments, the determining the intracranial pressure and/or the baseline time-based intracranial pressure changes comprises: applying a trained machine learning algorithm to the received vibroacoustic data and the electric potential data.

In certain embodiments, the method further comprises receiving, and applying the trained machine learning algorithm, to one or more of: temperature data of the subject; movement data of a body part of the subject (such as the chest); and a volatile organic compound data from the subject, to determine one or both of the intracranial pressure of the subject and the baseline time-based intracranial pressure changes. The temperature data may include temperature, or changes in temperature, of air flowing through a nose or a mouth of the subject.

In certain embodiments, the method further comprises identifying, from the determined intracranial pressure any intracranial pressure changes relative to the baseline time-based intracranial pressure changes, and determining presence of a condition in the subject by applying a trained machine learning algorithm to the intracranial pressure changes.

In certain embodiments, the method further comprises receiving, and applying the trained machine learning algorithm, to one or more of: temperature data of the subject; movement data of a body part of the subject; and a volatile organic compound data from the subject, to determine the presence of the condition. The temperature data may include temperature, or changes in temperature, of air flowing through a nose or a mouth of the subject.

In certain embodiments, the method further comprises determining or applying a treatment for the determined condition.

From a yet further aspect, there is provided a method for monitoring an intracranial pressure of a subject, the method executable by a processor of a computer system, the method comprising: receiving vibroacoustic data from a vibroacoustic sensor configured to non-invasively detect vibroacoustic signals associated with the subject within a bandwidth ranging from about 0.01 Hz to about 20 kHz, the vibroacoustic data having been collected from the subject over at least one heart cycle of the subject; receiving electric potential data from an electric potential sensor, the electric potential data having been collected non-invasively from the subject over the at least one heart cycle of the subject; determining, using the received vibroacoustic data, intracranial pressure of the subject; and determining, using the received electric potential data, baseline time-based events in the subject and portions of the vibroacoustic data corresponding to the baseline time-based events, determining occurrence of a change in the intracranial pressure due to a condition not related to the baseline time-based event by identifying portions of the vibroacoustic data not related to the baseline time-based events.

In certain embodiments, the method further comprises determining or applying a treatment for the determined condition.

From another aspect, there is provided a device comprising: a housing configured to be worn on a head, face, torso or neck of a subject: at least one sensor, housed in the housing, for detecting a vibroacoustic signal associated with the subject; and at least one stimulator, housed in the housing, for providing a vibroacoustic signal to the subject. A stimulator is any sensor or device that can emit a signal that can stimulate the subject.

In certain embodiments, the device further comprises at least one bioelectric sensor, housed in the housing, for detecting a bioelectric signal associated with the subject.

In certain embodiments, the housing is configured as a curved band that can be positioned at least partially around the head, face or neck of the subject.

In certain embodiments, the curved band has two free ends, one or both of the at least one sensor and the at least one stimulator being positioned in at least one of the two free ends.

In certain embodiments, the at least one sensor comprises two voice coil sensors spaced from one another in the housing.

In certain embodiments, the housing is sized and shaped to be positioned on the subject such that the vibroacoustic signal is provided to one or more of: an ear of the subject, a skull of the subject, the spine of the subject, the torso of a subject, a vagal nerve of the subject, a carotid artery of the subject.

From another aspect, there is provided a system comprising: a processor of a computer system, a device as described herein, wherein the processor is communicatively couplable to the at least one sensor and/or the at least one stimulator and is configured to control one or both of the at least one sensor and/or the at least one stimulator.

In certain embodiments, the processor is configured to determine the vibroacoustic signal to be provided by the stimulator to the subject.

In certain embodiments, the determining the vibroacoustic signal to be provided by the stimulator to the subject is based on a frequency-response function of the subject associated with one or more of damping, resonant and reflective responses of the subject to given frequencies.

In certain embodiments, the processor is configured to cause the at least one stimulator to apply vibroacoustic signals to the subject having different frequencies/intensities/durations/directions, and to measure a response of the subject to the different frequencies, optionally the response being one or more of: a bioelectric signal of a brain of the subject, a direct user input of the subject, a detected vibroacoustic signal of the subject.

In certain embodiments, the processor is configured to correlate the response of the subject with the different frequencies/intensities/durations/directions in order to compile a subject-specific library of signals.

In certain embodiments, the determining the vibroacoustic signal to be provided by the stimulator to the subject comprises the processor correlating a response of the subject to different frequencies/intensities/durations/directions of applied vibroacoustic signals.

In certain embodiments, the processor is configured to cause the stimulator to generate vibroacoustic signals comprising a sweep-frequency stimulation with a bandwidth of about 0.01 Hz to 80 kHz.

In certain embodiments, the processor is configured to cause the stimulator to generate vibroacoustic signals comprising a binaural audio.

In certain embodiments, the binaural beat comprises a lower frequency signal and a higher frequency signal, the lower frequency signal and the lower frequency signal alternatingly applied to the right and left ear, of the subject, with the frequency of the alternation between the respective signals being applied to the left and right ears being from about 0.001 Hz to 0.005 Hz, about 0.005 to 0.01 Hz, about 0.01 Hz to 0.05 Hz, about 0.05 Hz to 0.1 Hz, about 0.1 Hz to 0.5 Hz, about 0.5 Hz to 1 Hz, about 1 Hz to 5 Hz, about 5 Hz to 50 Hz, about 50 Hz to 200 Hz, about 200 Hz to 500 Hz, or about 500 Hz to 1000 Hz.

In certain embodiments, the processor is configured to retrieve vibroacoustic signals to be applied to the subject from a binaural sound library.

In certain embodiments, the system further comprises an electronic device associated with the subject, communicatively couplable to the processor of the computer system, the processor and/or the electronic device configured to provide an input or an output to the electronic device and/or the processor respectively.

From another aspect, there is provided device or plurality of devices configured to be coupled to a head, torso, face or neck of a subject, or to be positioned proximate the head, torso, face or neck of the subject, the device comprising: at least one vibroacoustic sensor for detecting a vibroacoustic signal associated with the subject; and at least one bioelectric sensor for detecting a bioelectric signal associated with the subject.

In certain embodiments, the device further comprises one or both of: an infrared thermographic camera for detecting temperature changes associated with the subject; a machine vision camera; and augmented reality/Virtual reality device or systems for environment and context manipulation.

In certain embodiments, the device is configured such that it can be positioned at least partially around the head, face or neck of the subject.

In certain embodiments, the at least one vibroacoustic sensor comprises at least one voice coil sensor.

In certain embodiments, either one or both of the at least one bioelectric sensor and the at least one vibroacoustic sensor is configured to detect pressure changes in the cranium.

In certain embodiments, the device is configured as one or more of a head set, earplug, head band, mask, eyewear, scarf, headwear.

From another aspect, there is provided a system comprising: a processor of a computer system, a device as described herein, wherein the processor is communicatively couplable to the at least one vibroacoustic sensor and/or the at least one bioelectric sensor, and is configured to: receive data from the at least one vibroacoustic sensor and/or the at least one bioelectric sensor; process data from the at least one vibroacoustic sensor and/or the at least one bioelectric sensor; control the at least one vibroacoustic sensor and/or the at least one bioelectric sensor; and/or provide an output related to the received data and/or the processed data; train a machine learning algorithm based at least in part on the received data and/or the processed data.

In certain embodiments, the processor is configured to train a machine learning algorithm based on intracranial pressure changes, electric potential changes and vibroacoustic changes of the subject.

In certain embodiments, the processor is configured to determine an intent of the subject based on the received data, and optionally wherein the intent is a word, a thought, a command, and optionally wherein the intent is determined without a direct input from the subject such as a vocalization, a gesticulation, or a written version of the intent.

In certain embodiments, the system further comprises an electronic device associated with the subject, communicatively couplable to the processor of the computer system, the processor and/or the electronic device configured to provide an input or an output to the electronic device and/or the processor respectively.

From a further aspect, there is provided a method, executable by a processor of a computer system, the method comprising: obtaining a data set including measured data associated with the subject, the data relating to one or more of vibroacoustic signals of the subject, bioelectric signals of the subject, temperature of the subject (e.g. a temperature of nasal or oral airflow), a flow rate of a breath of the subject, the data set including labels associated with an intent of the subject; training a machine learning algorithm on the data set and the labels, wherein the trained machine learning algorithm can predict a given intent of the subject, without an express expression of the intent by the subject, by applying the trained machine learning algorithm to detected signals of the subject, the detected signals comprising one or more of: vibroacoustic signals, bioelectric signals, temperature, a flow rate of a breath of the subject.

In certain embodiments, the intent is a word, a thought, a command, and optionally wherein the intent is determined without a direct input from the subject such as a vocalization, a gesticulation, or a written version of the intent.

From a yet further aspect, there is provided a method executable by a processor of a computer system, the method comprising: obtaining data associated with the subject, the data relating to one or more of vibroacoustic signals of the subject, bioelectric signals of the subject, temperature of the subject, and a flow rate of a breath of the subject; applying a trained machine learning algorithm to the data to predict a given intent of the subject without an express expression of the intent by the subject.

In certain embodiments, the intent is a word, a thought, a command, and optionally wherein the intent is determined without a direct input from the subject such as a vocalization, a gesticulation, or a written version of the intent.

In certain aspects, there is provided systems and methods for one or more of diagnosing, screening or treating for certain conditions such as a viral infection, carotid and coronary artery disease, and heart failure.

In certain aspects, there is provided systems and methods for one or more of promoting well being, reducing anxiety, inducing relaxation, stimulating creativity, managing pain, and increasing concentration.

In certain aspects, there is provided systems and methods for one or more of traumatic brain injury detection, vagal nerve observe, orient, decide and act (OODA) loop stimulation, gastric and bladder OODA loop stimulation, and placenta and uterus OODA loop stimulation.

In the context of the present specification, unless expressly provided otherwise, by subject is meant an animal.

In the context of the present specification, unless expressly provided otherwise, by animal is meant an individual animal that is a mammal, bird, or fish. Specifically, mammal refers to a vertebrate animal that is human and non-human, which are members of the taxonomic class Mammalia. Non-exclusive examples of non-human mammals include companion animals and livestock. Animals in the context of the present disclosure are understood to include vertebrates. The term vertebrate in this context is understood to comprise, for example fishes, amphibians, reptiles, birds, and mammals including humans. As used herein, the term “animal” may refer to a mammal and a non-mammal, such as a bird or fish. In the case of a mammal, it may be a human or non-human mammal. Non-human mammals include, but are not limited to, livestock animals and companion animals. As used herein, the term “plant” may refer to woody plants, such as trees, shrubs and other plants that produce wood as its structural tissue and thus has a hard stem. Other plants may include, but are not limited to food crops such as grasses, legumes, tubers, leafy vegetables, brassica, root vegetables, gourd, fungi, pods and other seed, fruit, flower, bulb, stem, leaf and nut bearing crops.

In the context of the present specification, unless expressly provided otherwise, the terms “audible” and “inaudible” relate to sounds within the audible and inaudible range, respectively, of the average human ear.

### BRIEF DESCRIPTION OF THE DRAWINGS

For a better understanding of the present technology, as well as other aspects and further features thereof, reference is made to the following description which is to be used in conjunction with the accompanying drawings, where:

FIGS. 1A, 1B and 1C show perspective, exploded and cross-sectional views, respectively, of a voice coil sensor for use in systems, methods and/or devices in accordance with various embodiments of the present technology.

FIGS. 2 and 3 show inner components of other voice coil sensors for use in systems, methods and/or devices in accordance with various embodiments of the present technology.

FIGS. 4A and 4B show plan and cross-sectional views of a piezoelectric sensor for use in systems, methods and/or devices in accordance with various embodiments of the present technology.

FIG. 5 shows a side view of a foldable sensor device for use in systems, methods and/or devices in accordance with various embodiments of the present technology.

FIGS. 6-9 show different wearable devices including one or more sensors in accordance with various embodiments of the present technology.

FIGS. 10 and 11 show wearable devices including one or more sensors and an augmented/virtual reality headpiece in accordance with various embodiments of the present technology.

FIGS. 12 and 13 show wearable devices, in the form of an earpiece, and including one or more sensors in accordance with various embodiments of the present technology.

FIG. 14 shows a wearable device, in the form of an eye-and-head piece and including one or more sensors in accordance with various embodiments of the present technology.

FIG. 15 shows a wearable device, in the form of a head piece, and including one or more sensors in accordance with various embodiments of the present technology.

FIGS. 16 and 17 show wearable devices, in the form of a face mask, and including one or more sensors in accordance with various embodiments of the present technology.

FIGS. 18A, 18B, 19, and 20 show wearable devices, in the form of an ear-head piece, and including one or more sensors in accordance with various embodiments of the present technology.

FIG. 21 shows a wearable device, in the form of an earpiece, and including one or more sensors and a speaker in accordance with various embodiments of the present technology.

FIG. 22 shows a system including a plurality of wearable devices in accordance with various embodiments of the present technology.

FIG. 23 is a flow diagram of a method for monitoring intracranial pressure in accordance with various embodiments of the present technology;

FIG. 24 is a flow diagram of a method for applying vibroacoustic signals and recording a response in accordance with various embodiments of the present technology;

FIG. 25 is a flow diagram of a method for determining an intracranial pressure in accordance with various embodiments of the present technology; and

FIG. 26 is a block diagram of an example computing environment in accordance with various embodiments of the present technology.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified embodiments of the present technology. As persons skilled in the art would understand, various embodiments of the present technology may be of greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and embodiments of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry and/or illustrative systems embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor,” may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some embodiments of the present technology, the processor may be a general purpose processor, such as a central processing unit (CPU) or a processor dedicated to a specific purpose, such as a digital signal processor (DSP). Moreover, explicit use of the term a “processor” should not be construed to refer exclusively to hardware capable of executing software, and may implicitly include, without limitation, application specific integrated circuit (ASIC), field programmable gate array (FPGA), read-only memory (ROM) for storing software, random access memory (RAM), and non-volatile storage. Some or all of the functions described herein may be performed by a cloud-based system. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown. Moreover, it should be understood that one or more modules may include for example, but without being limitative, computer program logic, computer program instructions, software, stack, firmware, hardware circuitry, or a combination thereof.

### Systems

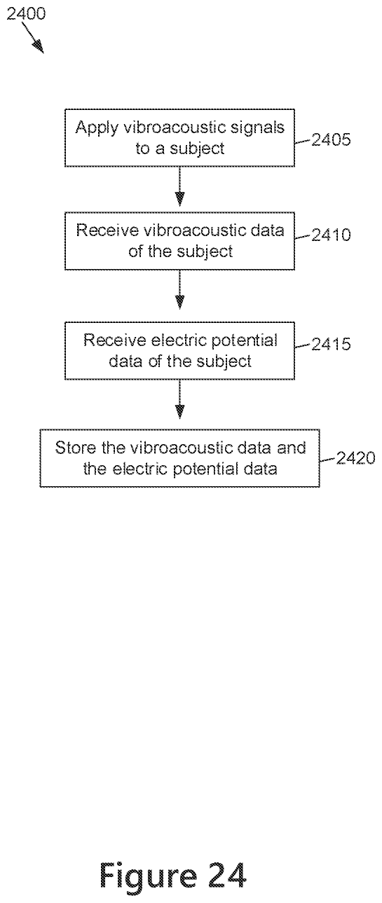

In certain aspects, and referring to FIG. 24, systems of the present technology comprise one or more sensors which may be embodied one or more devices.

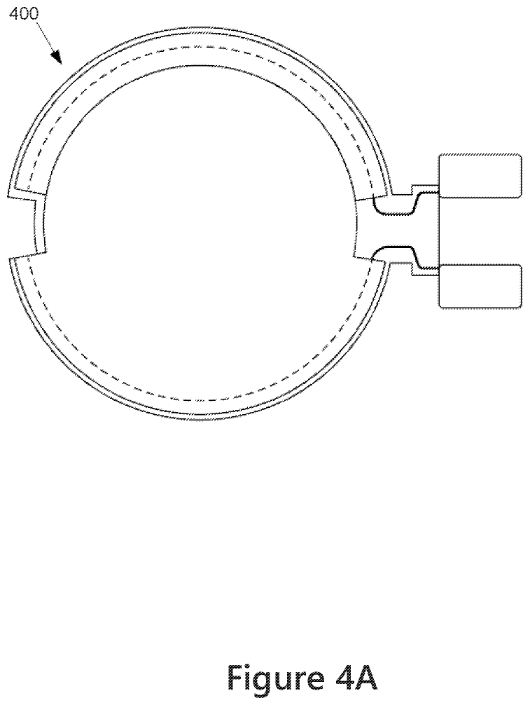

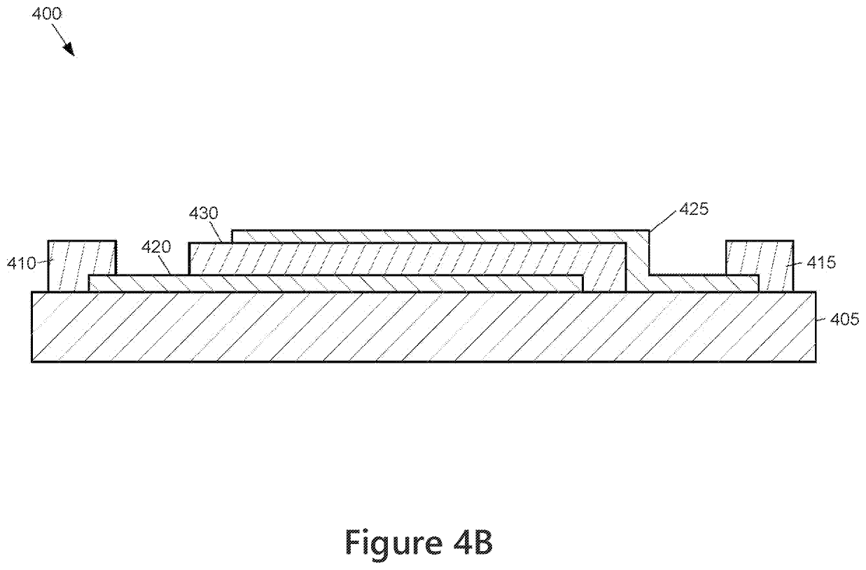

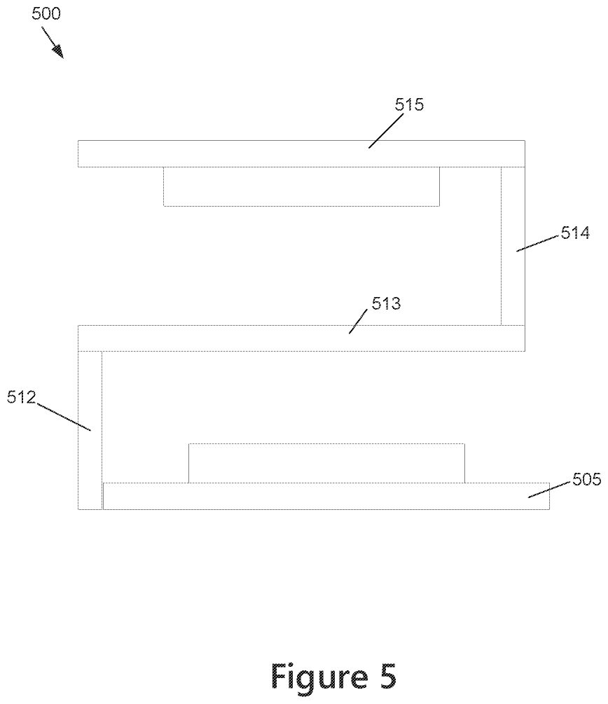

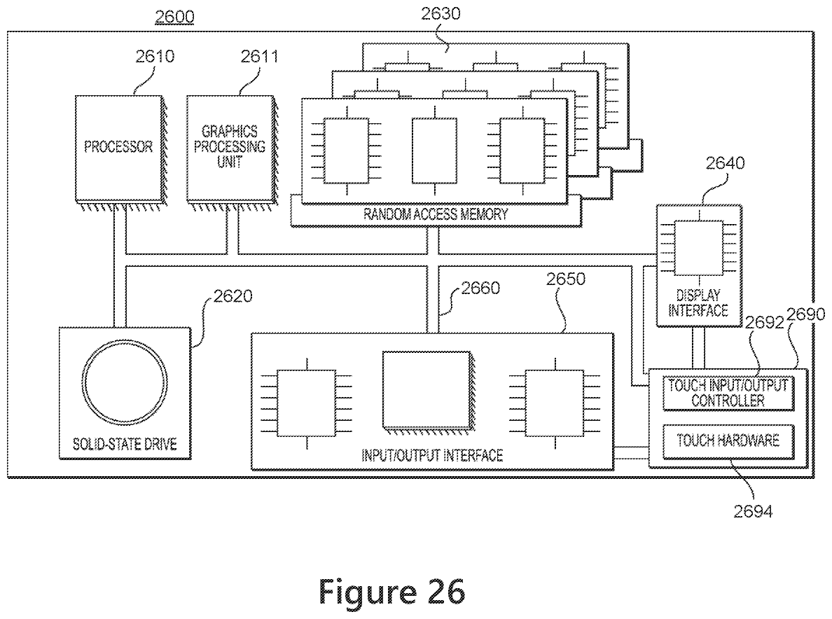

FIG. 24 illustrates a system 2440 for implementing and/or executing any of the devices and/or methods described herein such as for example determining an intent and/or an intracranial pressure of a user 2410 in an environment 2420. In certain embodiments, the system comprises a first wearable device 2411, including one or more sensors in a sensor device 2412, a second wearable device 2413, including one or more sensors in a sensor device 2414, and/or a third device 2415 including one or more sensors in a sensor device 2416. The first wearable device 2411, second wearable device 2413, and/or third device 2415 are communicatively coupled to a processor 2910 of a computing environment 2600 (further illustrated in FIG. 26) via a network 2430. The sensor devices 2412, 2414, and/or 2416 may comprise a multi-layer sensor device 2412, such as one of the devices illustrated in FIGS. 4A, 4B, and 5, and/or any other type of sensor device.

The first wearable device 2411 and/or second wearable device 2413 may be worn by the user 2410. For example the first wearable device 2411 may be a watch and the second wearable device 2413 may be a flexible patch. The sensor devices 2412 and 2414 may record data about the user 2410 and/or the environment 2420 surrounding the user 2410. The third device 2415 may be positioned within the environment 2420. For example the third device 2415 may be attached to a building, tower, and/or other structure. The sensor device 2416 may contain sensors that measure the environment 2420 surrounding the user 2410. The first wearable device 2411, second wearable device 2413, and/or third device 2415 may simultaneously record data about the user 2410 and/or environment 2420. Timestamped data may be collected from each of the first wearable device 2411, second wearable device 2413, and/or third device 2415. A location of each of the first wearable device 2411, second wearable device 2413, and/or third device 2415 may be determined. A distance between each of the first wearable device 2411, second wearable device 2413, and/or third device 2415 may be determined, such as by measuring the time-of-flight of communications transmitted between the devices.

In other embodiments (not shown), there may be provided the first wearable device 2411 including the one or more sensors in the sensor device 2412, without provision of the second wearable device 2413 and the third device 2415. The computing environment 2600 may be a standalone device (as illustrated) and/or integrated within the first wearable device 2411, second wearable device 2413, and/or third device 2415. The computing environment 2600 may be integrated in an intelligence coordinator device (e.g. a microcontroller). The intelligence coordinator device may gather data from multiple sensor devices and/or other devices. Individual devices may send alerts to the intelligence coordinator device, such as after detecting an anomalous event.

In yet other embodiments (not shown), there may be provided additional wearable devices or other devices with sensors communicatively coupled to the processor 2610 of the computing environment 2600 via the network 2430.

The environment 2420 may include the user 2410 and/or other users (not illustrated). Data about the other users and/or about the environment may be collected, such as by wearable devices being worn by the other users. Data collected about the other users in the environment 2420 may be collected by the computing environment 2600 and processed as environmental data corresponding to the user 2400. In other words, the data collected from the other users in the environment 2420 may be used as data describing the environment 2420. In certain embodiments, the system includes a wearable device which is configured to be non-invasively coupled to a head of a subject. The device may have any suitable form factor permitting its positioning proximate to or on the head of the subject or a portion of the head of the subject. Example configurations of embodiments of the device comprise: an earpiece which can be positioned over or at least partially within the ear, an eye-piece which can be positioned over at least one eye, or a head-piece which at least covers a part of the subject's head or neck. The wearable device for the head may also have a band-aid or patch configuration.

In certain embodiments, the system further includes a wearable device which is configured to be coupled to a part of the body of the subject other than the head, such as chest, back, wrist, arm, hand, ankle, leg, or foot of a subject. The device may have a band-aid form factor or be configured as a watch or wrist.

The system may include different numbers and combinations of the wearable devices for the head and wearable devices for body parts other than the head. For example, in certain embodiments, the system may include one wearable device in the form of a headpiece and a plurality of band-aid or patch configured devices attachable to the neck over the carotid artery and to the chest, for example. The sensors used in the wearable device(s) may include sensors for detecting and/or monitoring one or more of: acoustic signals from the subject, electric potential perturbations associated with movements of the subject or the subject's body parts, volatile organic compounds inhaled or exhaled by the subject, images of the subject and a temperature of the subject.

In certain embodiments, the system of the present technology comprises one or more remote devices configured for use remote from the subject. The remote device may be configured to emit a signal to the subject such as a sound, an image, and/or a haptic signal. The remote device may have a tablet form and include a display and/or a microphone for emitting the signal to the subject. The remote device may include one or more sensors for remotely capturing data from the subject such as acoustic data, temperature, images, and/or electric potential perturbations.

In certain embodiments, the system may include a computer system including a processor for receiving, sending and/or processing data to and/or from any one or more of the devices and/or systems.

### Sensors

Example sensors for inclusion in the systems, devices and methods of the present technology are described below.

### Vibroacoustic Sensors

Vibroacoustic sensor technologies provided by embodiments of the devices, methods and systems of the present technology were specifically engineered to capture a broad range of physiologically-relevant vibrations, including those that are inaudible and audible to the human ear. Example vibroacoustic sensors are described in U.S. Pat. No. 11,240,579 granted Feb. 1, 2022, WO 2021/224888 published Nov. 11, 2021 and PCT/US21/46566 filed Aug. 18, 2021, the contents of which of each are herein incorporated by reference in their entirety.

The human ear can hear sound waves that have a frequency of about 20-20,000 hertz (Hz, cycles/second). Ultrasound refers to waves that have a frequency higher than about 20,000 Hz and are therefore outside the human hearing range. Infrasound refers to waves that have a frequency less that than about 20 Hz and are therefore also outside the human hearing range.

The threshold of human audibility decreases sharply as vibrational frequency falls below about 500 Hz. However, in a healthy subject at rest, most cardiac, respiratory, digestive, and movement-related information is inaudible to humans, as this information occurs at frequencies below those associated with speech. Thus, the majority of bodily vibrations are neither detected nor included in conventional diagnostic medical practices due to the low frequency band of these vibrations, and the limited bandwidth limits of conventional instruments (e.g., conventional stethoscopes). Variations of the system described herein are capable of detecting, amplifying and analyzing a broad spectrum of infrasound, ultrasound, and far-ultrasound vibroacoustic frequencies, and are thus advantageous for a more comprehensive, holistic picture of subject health and condition.

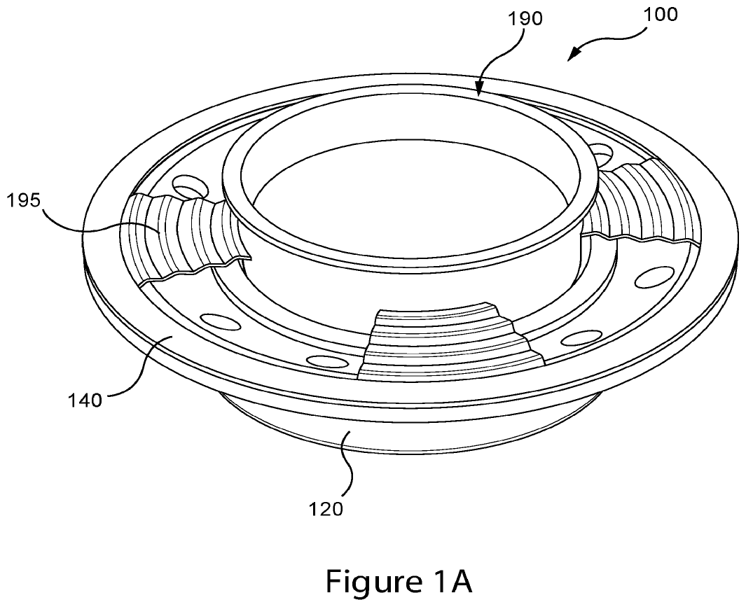

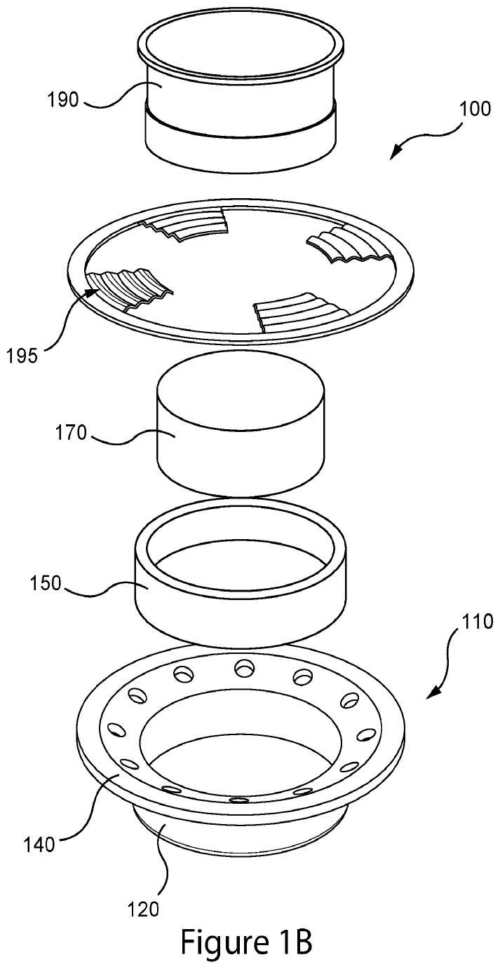

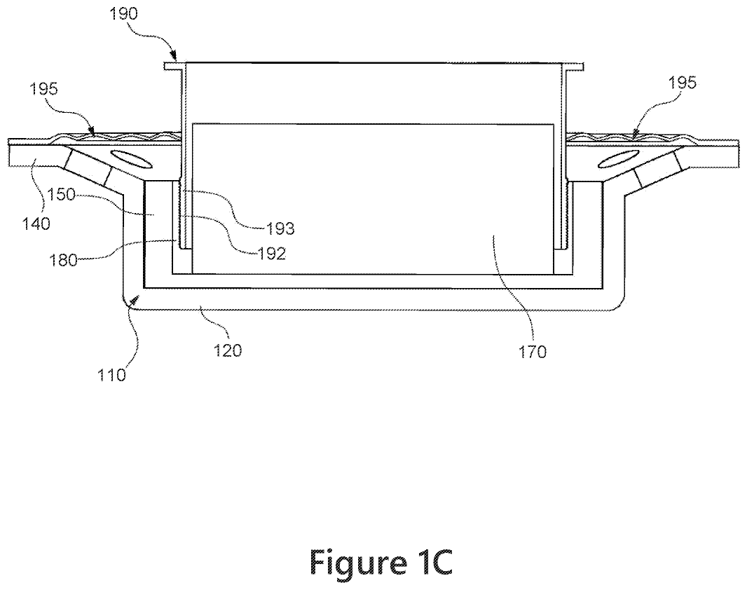

In certain embodiments, the vibroacoustic sensor is of a voice coil transducer type. For example, and referring to FIGS. 1A, 1B and 1C, a voice coil transducer 100 is shown which comprises a frame 110 (also referred to as a surround pot) having a cylindrical body portion 120 with a bore 130, and a flange 140 extending radially outwardly from the cylindrical body portion. The frame may be made of steel. An iron core 150 such as soft iron or other magnetic material is attached to the cylindrical body portion and lines the bore of the cylindrical body portion. The iron core extends around the bore of the cylindrical body portion as well as across an end of the cylindrical body portion. The iron core has an open end. A magnet 170 is positioned in the bore and is surrounded by, and spaced from, the iron core to define a magnet gap 180. A voice coil 190, comprising one or more layers of wire windings 192 supported by a coil holder 193, is suspended and centered in relation to the magnet gap by one or more spiders 195. The wire windings may be made of a conductive material such as copper or aluminum. A periphery of the spider is attached to the frame, and a center portion is attached to the voice coil. The voice coil at least partially extends into the magnet gap through the open end of the iron core. The one or more spiders 195 allow for relative movement between the voice coil and the magnet whilst minimizing or avoiding torsion and in-plane movements. A diaphragm may be provided which may be attached to the voice coil transducer. In steady state, when no pressure is being applied to the diaphragm, the voice coil may be positioned such that it is not fully received in the magnet gap (off-center in respect to optimal placement within the magnet gap). In use, the voice coil can be pushed into the magnet gap to center it when pressure is applied to the diaphragm under normal use. A dust cap may be provided over the open end to prevent foreign object access. An outer cover (not shown) may be provided on top of the diaphragm to seal any openings between the diaphragm and the housing. The outer cover may be made of an elastomeric material such as rubber.

In use, the voice coil transducer can be used to detect acoustic signals of the subject by either coupling the diaphragm to skin, such as in the ear, face, neck or scalp of the subject; clothing of the subject; hair of the subject; or by positioning the subject and the diaphragm proximate to one another. Movements induced in the acoustic waves will cause the diaphragm to move, in turn inducing movement of the voice coil within the magnet gap, resulting in an induced electrical signal.

In certain variations of the voice coil transducer, the configuration of the transducer is arranged to pick up more orthogonal signals than in-plane signals, thereby improving sensitivity. For example, the one or more spiders 195 are designed to have out-of-plane compliance and be stiff in-plane. The same is true of the diaphragm whose material and stiffness properties can be selected to improve out-of-plane compliance. The diaphragm may have a convex configuration (e.g., dome shaped) to further help in rejecting non-orthogonal signals by deflecting them away. Furthermore, signal processing may further derive any non-orthogonal signals e.g., by using a 3 axis accelerometer. This either to further reject non-orthogonal signals or even to particularly allow non-orthogonal signals through the sensor to derive the angle of origin of the incoming acoustic wave.

The sensitivity and different noise/signal ratios challenges can be adapted certain variables can be modulated to optimize the voice coil transducer for the specific intended use: magnet strength, magnet volume, voice coil height, wire thickness, number of windings, number of winding layers, winding material (e.g., copper vs aluminum), and spider configuration.

In certain variations, the voice coil is configured to have an impedance of more than about 10 Ohms, more than about 20 Ohms, more than about 30 Ohms, more than about 40 Ohms, more than about 50 Ohms, more than about 60 Ohms, more than about 70 Ohms, more than about 80 Ohms, more than about 90 Ohms, more than about 100 Ohms, more than about 110 Ohms, more than about 120 Ohms, more than about 130 Ohms, more than about 150 Ohms, or about 150 Ohms. This is higher than a conventional heavy magnet voice coil transducer which has an impedance of about 4-8 Ohms. This is achieved by modulating one or more of the number of windings, wire diameter, and winding layers in the voice coil. Many permutations of these parameters are possible, and have been tested by the developers, as set out in Example 6. In one such variation, the voice coil comprises fine wire and was configured to have an impedance of about 150 Ohms, and associated lowered power requirement, by increasing the wire windings.

Developers also discovered that adaptation of the configuration of the spider contributed to increasing sensitivity and signal/noise ratio increases. More specifically, it was determined via experiment and simulation that making the spider more compliant such as by incorporating apertures in the spider, increased sensitivity. Apertures also allow for free air flow.

In certain variations, a single voice coil transducer of the current technology can provide a microphonic frequency response of less than about 1 Hz to over about 150 kHz or about 0.01 Hz to about 160 kHz

Furthermore, the use of such a voice coil transducer also enables a size to be kept to a practical minimum which is useful for wearable and head-piece applications.

In certain variations of the present technology, the voice coil transducer comprises a single layer of spider. In certain other variations of the present technology, the voice coil transducer comprises a double layer of the spider. Multiple spider layers comprising three, four or five layers, without limitation, are also possible.

Instead of a one-piece corrugated continuous configuration as is known in conventional spiders of conventional voice coils, in certain variations of the current technology, the spider has a discontinuous surface. The spider may comprise at least two deflecting structures which are spaced from one another, permitting air flow therebetween. In certain configurations, the deflecting structures comprises two or more arms extending radially, and spaced from one another, from a central portion of the spider, such as four arms extending radially from the central portion. The four arms increase in width as they extend outwardly. Each of the arms has a corrugated configuration. An aperture between each of the arms is larger than an area of each deflecting arm.

Other variants of the spider for the voice coil transducer comprise a deflecting structure comprising one or more arms extending from a central portion and defining apertures therebetween. The one or more arms may be straight or curved. The one or more arms may have a width which varies along its length, or which is constant along its length. The one or more arms may be configured to extend from the central portion in a spiral manner to a perimeter 840 of the spider. A solid ring may be provided at the perimeter of the spider. In certain variations, there may be provided a single arm configured to extend as a spiral from the central portion of the spider to the perimeter of the spider. In these cases, turns of the spiral arms define the apertures. The spider may be defined as comprising a segmented form including portions that are solid (the arm(s)) and portions which are the aperture(s) defined therebetween. The arms may be the same or different. In variants where more than one layer of the spider is provided in the voice coil transducer, the spiders of each layer may be the same or different.

The configuration chosen for a given use of the device will depend on the amount of compliance required for that given use. For example, a voice coil configuration of low compliance may be chosen for contact applications than non-contact applications. For contact applications, spider may be coupled to the voice coil in such a way as to off-set the voice coil from the magnet gap when there is no pressure applied to the diaphragm, and when the expected pressure is applied to the diaphragm, the voice coil will be pushed into the magnet gap for optimum positioning and acoustic signal detection.

In certain variations, a compliance of the diaphragm may range from about 0.4 to 3.2 mm/N. The compliance range may be described as low, medium and high, as follows: 0.4 mm/N: low compliance->fs around 80-100 Hz; 1.3 mm/N: medium compliance->fs around 130 Hz; and 3.2 mm/N: high compliance->fs around 170 Hz.

In some variations, two or more voice coil sensors may be included in the device which may enable triangulation of faint body sounds detected by the voice coil sensors, and/or to better enable cancellation and/or filtering of noise such as environmental disturbances. Sensor fusion data of two or more voice coil sensors can be used to produce low resolution sound intensity images.

In some variations, the voice coil transducer may be optimized for vibroacoustic detection, such as by using non-conventional voice coil materials and/or winding techniques. For example, in some variations, the voice coil material may include aluminum instead of conventional copper. Although aluminum has a lower specific conductance, overall sensitivity of the voice coil transducer may be improved with the use of aluminum due to the lower mass of aluminum. Additionally, or alternatively, the voice coil may include more than two layers or levels of winding (e.g., three, four, five, or more layers or levels), in order to improve sensitivity. In certain variants, the wire windings may comprise silver, gold or alloys for desired properties. Any suitable material may be used for the wire windings for the desired function. In certain other variants, the windings may be printed, using for example conductive inks onto the diaphragm.

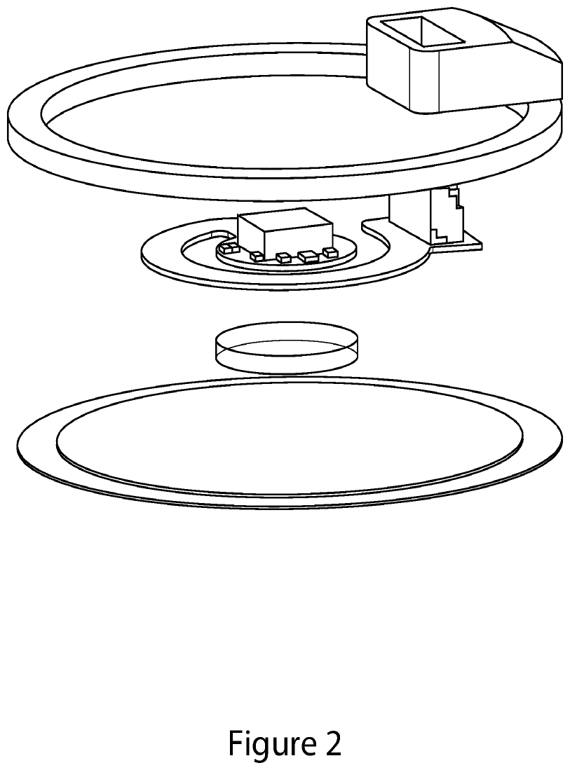

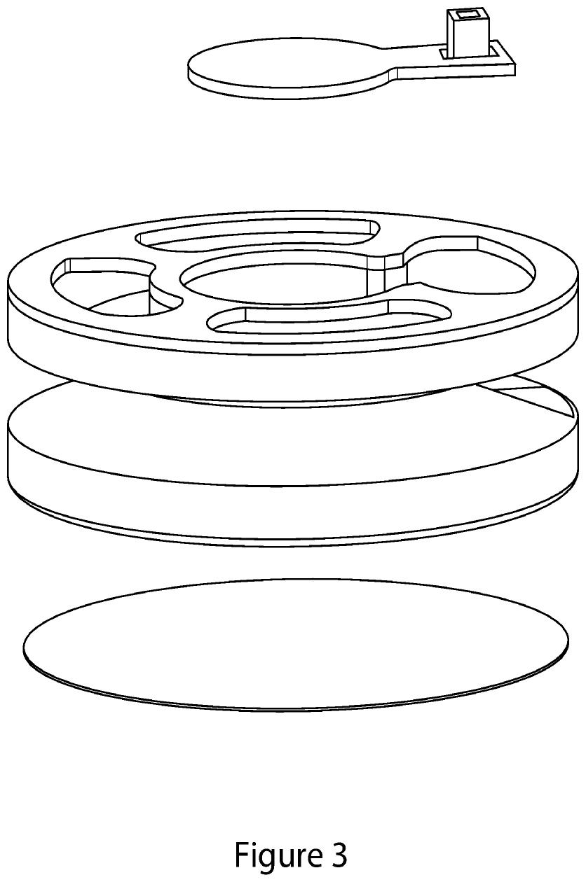

FIGS. 2 and 3 show alternative embodiments of the voice coil transducer of FIGS. 1A, 1B and 1C.

### Electric Potential Sensor

Electric potential sensors that can be used with the current technology are not particularly limited. In certain embodiments, the electric potential sensor is an active ultrahigh impedance capacitively coupled sensor.

An example electric potential sensor for use in the present technology comprises one or more Electric Potential Integrated Circuit (EPIC) sensors that allow non-contact, at a distance and through-clothing measurements. Certain EPIC sensors used within present systems and devices may include one or more as described in: U.S. Pat. Nos. 8,923,956; 8,860,401; 8,264,246; 8,264,247; 8,054,061; 7,885,700; the contents of which are herein incorporated by reference.

An example EPIC sensor comprises layers of an electrode, a guard and a ground. A circuit is positioned on top of the ground. The electrode may have an optional resist layer.

Electric Potential sensors (EPS) can pick up subtle movement of nearby objects due to the disturbance of static electric fields they cause. An EPS close to the diaphragm of a voice coil transducer is hence able to sense the motion of the vibrating diaphragm. In contrast to the voice coil-based sensor, the electric potential sensor may not add significant mass or additional spring constant and hence can maintain the original compliance of the diaphragm thereby avoiding a potential reduction in sensitivity.

In certain embodiments, the absence of 1/f noise makes the EPIC sensor ideal for use with signal frequencies of −10 Hz or less. EPS can be used to measure standard electrocardiogram (ECG), electroencephalogram (EEC), electromyogram (EMG), galvanic skin response, or impedance cardiography.

The electric potential sensor can be used to detect chest movement and nostril movement of the subject to determine breath rates, for example, as well as facial muscle motion.

### Piezoelectric Sensor

In certain embodiments, the systems, devices and methods of the present technology include one or more sensors with a piezoelectric component which can be used to detect acoustic signals and/or electric potential signals. An example piezoelectric-based transducer has been described and illustrated in PCT/US21/59193 filed Nov. 12, 2021, the contents of which are herein incorporated by reference.

Referring to FIGS. 4A and 4B, the piezoelectric transducer 400 comprises a substrate layer 405, a first electrode layer 420 on the substrate layer, a first piezoelectric layer 430 on the first electrode layer, a second electrode layer 425 on the first piezoelectric layer, a first electrical connector 410 connected to the first electrode and a second electrical connector 415 connected to the second electrode, one or both of the first electrical connector and the second electrical connector being connectable to an electronics circuit or to a ground. The electronics circuit may be any suitable electronics circuit for collecting signals from the first and second electrodes.

The transducer can function as an electrical potential sensor when the first piezoelectric layer is not polarized. In this arrangement, in which the piezoelectric layer 430 is not polarized, the piezoelectric layer 430 may act as an insulator between the two electrode layers 420 and 425. The transducer can function as an acoustic sensor when the first piezoelectric layer is polarized. The transducer 400 acting as an acoustic sensor may operate via a piezoresistive and/or optical force modality. The transducer 400 can be used to detect a pressure wave generated by blood flow in the carotid artery, for confirming a heartrate of the user.

The substrate can be flexible and/or elastic. During use of the transducer 400, the substrate layer may be placed against the user's skin, held close to the skin or be incorporated in a piece of clothing, headwear, footwear, eyewear, accessory, blanket, band-aid, bandage or the like. Accordingly, the substrate layer 405 may be made of a biocompatible material that will not irritate or otherwise damage the user's skin.

The transducer 400 may be printed on the substrate layer 405, such as using a screen-printing and/or ink-jet printing process. Using a screen-printing and/or ink-jet printing process may optimize and/or increase flexibility, performance, and product reliability. The first electrode layer 420 may be formed on the substrate layer 405 such as by printing. The piezoelectric layer 430 may be formed on the first electrode layer 420 such as by printing. The second electrode layer 425 may be formed on the piezoelectric layer 430 such as by printing.

The first electrode layer 420 and/or second electrode layer 425 may have a thickness of about 100 to about 600 nm. The first electrode layer 420 and second electrode layer 425 may have a same thickness, such as about 400 nm. In other embodiments, the first electrode layer 420 and the second electrode layer 425 may have a different thickness. The piezoelectric layer 430 is positioned between the first electrode layer 420 and the second electrode layer 425. The piezoelectric layer 430 is in contact with the first electrode layer 420 and the second electrode layer 425. The piezoelectric layer 430 may have a thickness of about 4 to about 10 μm. A variation of the thickness of the piezoelectric layer (in other words a surface roughness) may be less than about 2000 nm, or less than about 1000 nm.

### Acoustocardiography (ACG) Sensor

In certain embodiments, the systems, devices and methods of the present technology include one or more acoustic cardiography (ACG) sensors for detecting vibrations of the heart as the blood moves through the various chambers, valves, and large vessels. The ACG sensor can record these vibrations at four locations of the heart and provides a “graph signature.” While the opening and closing of the heart valves contributes to the graph, so does the contraction and strength of the heart muscle. As a result, a dynamic picture is presented of the heart in motion. The ACG is not the same as an ECG, which is a common diagnostic test. The electrocardiograph (ECG) records the electrical impulses as it moves through the nerves of the heart tissue as they appear on the skin. The ECG primarily indicates if the nervous tissue network of the heart is affected by any trauma, damage (for example from a prior heart attack or infection), severe nutritional imbalances, stress from excessive pressure. Only the effect on the nervous system is detected. It will not tell how well the muscle or valves are functioning, etc. In addition, the ECG is primarily used to diagnose a disease. The ACG sensor not only looks at electrical function but also looks at heart muscle function, which serves as a window of the metabolism of the entire nervous system and the muscles. Using the heart allows a “real-time” look at the nerves and muscles working together. As a result of this interface, unique and objective insights into health of the heart and the entire person can better be seen.

### Passive Acoustocerebrography (ACG) Sensor

In certain embodiments, the systems, devices and methods of the present technology include one or more passive acoustocerebrography sensors for detecting blood circulation in brain tissue. This blood circulation is influenced by blood circulating in the brain's vascular system. With each heartbeat, blood circulates in the skull, following a recurring pattern according to the oscillation produced. This oscillation's effect, in turn, depends on the brain's size, form, structure and its vascular system. Thus, every heartbeat stimulates minuscule motion in the brain tissue as well as cerebrospinal fluid and therefore produces small changes in intracranial pressure. These changes can be monitored and measured in the skull. The one or more passive acoustocerebrography sensors may include passive sensors like accelerometers to identify these signals correctly. Sometimes highly sensitive microphones can be used.

### Active Acoustocerebrography (ACG) Sensor

In certain embodiments, the systems, devices and methods of the present technology include one or more active acoustocerebrography sensors. Active ACG sensors can be used to detect a multi-frequency ultrasonic signal for classifying adverse changes at the cellular or molecular level. In addition to all of the advantages that passive ACG sensor provide, the active ACG sensor can also conduct a spectral analysis of the acoustic signals received. These spectrum analyses not only display changes in the brain's vascular system, but also those in its cellular and molecular structures. The active ACG sensor can also be used to perform a Transcranial Doppler test, and optionally in color. These ultrasonic procedures can measure blood flow velocity within the brain's blood vessels. They can diagnose embolisms, stenoses and vascular constrictions, for example, in the aftermath of a subarachnoid hemorrhage.

### Ballistocardiography (BCG) Sensor

In certain embodiments, the systems, devices and methods of the present technology include one or more ballistocardiograph sensor (BCG) for detecting ballistic forces generated by the heart. The downward movement of blood through the descending aorta produces an upward recoil, moving the body upward with each heartbeat. As different parts of the aorta expand and contract, the body continues to move downward and upward in a repeating pattern. Ballistocardiography is a technique for producing a graphical representation of repetitive motions of the human body arising from the sudden ejection of blood into the great vessels with each heartbeat. It is a vital sign in the 1-20 Hz frequency range which is caused by the mechanical movement of the heart and can be recorded by noninvasive methods from the surface of the body. Main heart malfunctions can be identified by observing and analyzing the BCG signal. BCG can also be monitored using a camera-based system in a non-contact manner. One example of the use of a BCG is a ballistocardiographic scale, which measures the recoil of the person's body who is on the scale. A BCG scale is able to show a person's heart rate as well as their weight.

### Electromyography (EMG) Sensor

In certain embodiments, the systems, devices and methods of the present technology include one or more Electromyography (EMG) sensor for detecting electrical activity produced by skeletal muscles. The EMG sensor may include an electromyograph to produce a record called an electromyogram. An electromyograph detects the electric potential generated by muscle cells when these cells are electrically or neurologically activated. The signals can be analyzed to detect medical abnormalities, activation level, or recruitment order, or to analyze the biomechanics of human or animal movement. EMG can also be used in gesture recognition.

### Electrooculography (EOG) Sensor

In some variations, the systems, devices and methods of the present technology include one or more electrooculography (EOG) sensors for measuring the corneo-retinal standing potential that exists between the front and the back of the human eye. The resulting signal is called the electrooculogram. Primary applications are in ophthalmological diagnosis and in recording eye movements. Unlike the electroretinogram, the EOG does not measure response to individual visual stimuli. To measure eye movement, pairs of electrodes are typically placed either above and below the eye or to the left and right of the eye. If the eye moves from center position toward one of the two electrodes, this electrode “sees” the positive side of the retina and the opposite electrode “sees” the negative side of the retina. Consequently, a potential difference occurs between the electrodes. Assuming that the resting potential is constant, the recorded potential is a measure of the eye's position.

### Electroolfactography (EOG) Sensor

In certain embodiments, the systems, devices and methods of the present technology include one or more Electro-olfactography or electroolfactography (EOG) sensors for detecting a sense of smell of the subject. The EOG sensor can detect changing electrical potentials of the olfactory epithelium, in a way similar to how other forms of electrography (such as ECG, EEG, and EMG) measure and record other bioelectric activity. Electro-olfactography is closely related to electroantennography, the electrography of insect antennae olfaction.

### Electroencephalography (EEG) Sensor

In certain embodiments, the systems, devices and methods of the present technology include one or more electroencephalography (EEG) sensors for electrophysiological detection of electrical activity of the brain to “listen” to the brain and capture subtle pressure and pressure gradient changes related to the speech processing circuitry. EEG is typically noninvasive, with the electrodes placed along the scalp, although invasive electrodes are sometimes used, as in electrocorticography. EEG measures voltage fluctuations resulting from ionic current within the neurons of the brain. Clinically, EEG refers to the recording of the brain's spontaneous electrical activity over a period of time, as recorded from multiple electrodes placed on the scalp. Diagnostic applications generally focus either on event-related potentials or on the spectral content of EEG. The former investigates potential fluctuations time locked to an event, such as ‘stimulus onset’ or ‘button press’. The latter analyses the type of neural oscillations (popularly called “brain waves”) that can be observed in EEG signals in the frequency domain. EEG can be used to diagnose epilepsy, which causes abnormalities in EEG readings. It can also used to diagnose sleep disorders, depth of anesthesia, coma, encephalopathies, and brain death. EEG, as well as magnetic resonance imaging (MRI) and computed tomography (CT) can be used to diagnose tumors, stroke and other focal brain disorders. Advantageously, EEG is a mobile technique available and offers millisecond-range temporal resolution which is not possible with CT, PET or MRI. Derivatives of the EEG technique include evoked potentials (EP), which involves averaging the EEG activity time-locked to the presentation of a stimulus of some sort (visual, somatosensory, or auditory). Event-related potentials (ERPs) refer to averaged EEG responses that are time-locked to more complex processing of stimuli.

### Ultra-Wideband (UWB) Sensor

In certain embodiments, the systems, devices and methods of the present technology include one or more ultra-wideband sensors (also known as UWB, ultra-wide band and ultraband). UWB is a radio technology that can use a very low energy level for short-range, high-bandwidth communications over a large portion of the radio spectrum. UWB has traditional applications in non-cooperative radar imaging. Most recent applications target sensor data collection, precision locating and tracking applications. A significant difference between conventional radio transmissions and UWB is that conventional systems transmit information by varying the power level, frequency, and/or phase of a sinusoidal wave. UWB transmissions transmit information by generating radio energy at specific time intervals and occupying a large bandwidth, thus enabling pulse-position or time modulation. The information can also be modulated on UWB signals (pulses) by encoding the polarity of the pulse, its amplitude and/or by using orthogonal pulses. UWB pulses can be sent sporadically at relatively low pulse rates to support time or position modulation, but can also be sent at rates up to the inverse of the UWB pulse bandwidth. Pulse-UWB systems have been demonstrated at channel pulse rates in excess of 1.3 gigapulses per second using a continuous stream of UWB pulses (Continuous Pulse UWB or C-UWB), supporting forward error correction encoded data rates in excess of 675 Mbit/s.

A valuable aspect of UWB technology is the ability for a UWB radio system to determine the “time of flight” of the transmission at various frequencies. This helps overcome multipath propagation, as at least some of the frequencies have a line-of-sight trajectory. With a cooperative symmetric two-way metering technique, distances can be measured to high resolution and accuracy by compensating for local clock drift and stochastic inaccuracy.

Another feature of pulse-based UWB is that the pulses are very short (less than 60 cm for a 500 MHz-wide pulse, and less than 23 cm for a 1.3 GHz-bandwidth pulse)—so most signal reflections do not overlap the original pulse, and there is no multipath fading of narrowband signals. However, there is still multipath propagation and inter-pulse interference to fast-pulse systems, which must be mitigated by coding techniques.

Ultra-wideband is also used in “see-through-the-wall” precision radar-imaging technology, precision locating and tracking (using distance measurements between radios), and precision time-of-arrival-based localization approaches. It is efficient, with a spatial capacity of about 1013 bit/s/m². UWB radar has been proposed as the active sensor component in an Automatic Target Recognition application, designed to detect humans or objects that have fallen onto subway tracks.

Ultra-wideband pulse Doppler radars can also be used to monitor vital signs of the human body, such as heart rate and respiration signals as well as human gait analysis and fall detection. Advantageously, UWB has less power consumption and a high-resolution range profile compared to continuous-wave radar systems. However, its low signal-to-noise ratio has made it vulnerable to errors.

In the USA, ultra-wideband refers to radio technology with a bandwidth exceeding the lesser of 500 MHz or 20% of the arithmetic center frequency, according to the U.S. Federal Communications Commission (FCC). A Feb. 14, 2002 FCC Report and Order authorized the unlicensed use of UWB in the frequency range from 3.1 to 10.6 GHz. The FCC power spectral density emission limit for UWB transmitters is −41.3 dBm/MHz. This limit also applies to unintentional emitters in the UWB band (the “Part 15” limit). However, the emission limit for UWB emitters may be significantly lower (as low as −75 dBm/MHz) in other segments of the spectrum. Deliberations in the International Telecommunication Union Radiocommunication Sector (ITU-R) resulted in a Report and Recommendation on UWB in November 2005. UK regulator Ofcom announced a similar decision on 9 Aug. 2007. More than four dozen devices have been certified under the FCC UWB rules, the vast majority of which are radar, imaging or locating systems.

### Seismocardiography (SCG) Sensor

In certain embodiments, the systems, devices and methods of the present technology include one or more seismocardiography (SCG) sensors for non-invasive measurement of cardiac vibrations transmitted to the chest wall by the heart during its movement. SCG can be used to observe changes in the SCG signal due to ischemia, cardiac stress monitoring, and assessing the timing of different events in the cardiac cycle. Using these events, assessing, for example, myocardial contractility might be possible. SCG has also been proposed to be capable of providing enough information to compute heart rate variability estimates. A more complex application of cardiac cycle timings and SCG waveform amplitudes is the computing of respiratory information from the SCG.

### Intracardiac Electrogram (IGM) Sensor

In certain embodiments, the systems, devices and methods of the present technology include one or more intracardiac electrogram (IGM) sensors for non-invasive measurement of cardiac electrical activity generated by the heart during its movement. It provides a record of changes in the electric potentials of specific cardiac loci as measured by electrodes placed within the heart via cardiac catheters; it is used for loci that cannot be assessed by body surface electrodes, such as the bundle of His or other regions within the cardiac conducting system.

### Pulse Plethysmograph (PPG) Sensor

In certain embodiments, the systems, devices and methods of the present technology include one or more pulse plethysmograph (PPG) sensors for non-invasive measurement of the dynamics of blood vessel engorgement. The sensor may use a single wavelength of light, or multiple wavelengths of light, including far infrared, near infrared, visible or UV. For UV light, the wavelengths used are between about 315 nm and 400 nm and the sensor is intended to deliver less than 8 milliwatt-hours per square centimeter per day to the subject during its operation.

### Galvanic Skin Response (GSR) Sensor

In certain embodiments, the systems, devices and methods of the present technology include one or more galvanic skin response (GSR) sensors. These sensors may utilize either wet (gel), dry, or non-contact electrodes as described herein.

### Volatile Organic Compounds (VOC) Sensors

In certain embodiments, the systems, devices and methods of the present technology include one or more volatile organic compounds (VOC) sensors for detecting VOC or semi-VOCs in exhaled breath of the subject. The potential of exhaled breath analysis is huge, with applications in many fields including, but not limited to, the diagnosis and monitoring of disease. Certain VOCs are linked to biological processes in the human body. For instance, dimethylsulfide is exhaled as a result of fetor hepaticus and acetone is excreted via the lungs during ketoacidosis in diabetes. Typically, VOC Excretion or Semi-VOC excretion can be measured using plasmon surface resonance, mass spectroscopy, enzymatic based, semiconductor based or imprinted polymer-based detectors.

### Vocal Tone Inflection (VTI) Sensors

In certain embodiments, the systems, devices and methods of the present technology include one or more vocal tone inflection (VTI) sensors. VTI analysis can be indicative of an array of mental and physical conditions that make the subject slur words, elongate sounds, or speak in a more nasal tone. They may even make the subject's voice creak or jitter so briefly that it's not detectable to the human ear. Furthermore, vocal tone changes can also be indicative of upper or lower respiratory conditions, as well as cardiovascular conditions.

### Capacitive Sensor

In certain embodiments, the systems, devices and methods of the present technology include one or more capacitive/non-contact sensors. Such sensors may include non-contact electrodes. These electrodes were developed since the absence of impedance adaptation substances could make the skin-electrode contact instable over time. This difficulty was addressed by avoiding physical contact with the scalp through non-conductive materials (i.e., a small dielectric between the skin and the electrode itself): despite the extraordinary increase of electrode impedance (>200 MOhm), in this way it will be quantifiable and stable over time.

A particular type of dry electrode, is known as a capacitive or insulated electrode. These electrodes require no ohmic contact with the body since it acts as a simple capacitor placed in series with the skin, so that the signal is capacitively coupled. The received signal can be connected to an operational amplifier and then to standard instrumentation.

The use of a dielectric material in good contact to the skin results in a fairly large coupling capacitance, ranging from 300 pF to several nano-farads. As a result, a system with reduced noise and appropriate frequency response is readily achievable using standard high-impedance FET (field-effect transistor) amplifiers.

While wet and dry electrodes require physical contact with the skin to function, capacitive electrodes can be used without contact, through an insulating layer such as hair, clothing or air. These contactless electrodes have been described generally as simple capacitive electrodes, but in reality there is also a small resistive element, since the insulation also has a non-negligible resistance.

The capacitive sensors can be used to measure heart signals, such as heart rate, in subjects via either direct skin contact or through one and two layers of clothing with no dielectric gel and no grounding electrode, and to monitor respiratory rate. High impedance electric potential sensors can also be used to measure breathing and heart signals.

### Capacitive Plates Sensor

In certain embodiments, the systems, devices and methods of the present technology include one or more capacitive plate sensors. The resistive properties of the human body may also be interrogated using the changes in dielectric properties of the human body that come with difference in hydration, electrolyte, and perspiration levels. The system or device may comprise two parallel capacitive plates which are positionable on either side of the body or body part to be interrogated. A specific time varying potential can be applied to the plates, and the instantaneous current required to maintain the specific potential is measured and used as input into the machine learning system to correlate the physiological states to the data. As the dielectric properties of the body or body part changes with resistance, the changes are reflected in the current required to maintain the potential profile.

### Machine Vision Sensor Module

In certain embodiments, the systems, devices and methods of the present technology include one or more one or more machine vision sensor modules comprising one or more optical sensors such as cameras for capturing the motion of the subject, or parts of the subject, as they stand or move (e.g. walking, running, playing a sport, balancing etc.). In this manner, physiological states that affect kinesthetic movements such as balance and gait patterns, tremors, swaying or favoring a body part can be detected and correlated with the other data obtained from the other sensors in the apparatus such as center of mass positioning. Machine vision allows skin motion amplification to accurately measure physiological parameters such as blood pressure, heart rate, and respiratory rate. For example, heart/breath rate, heart/breath rate variability, and lengths of heart/breath beats can be estimated from measurements of subtle head motions caused in reaction to blood being pumped into the head, from hemoglobin information via observed skin color, and from periodicities observed in the light reflected from skin close to the arteries or facial regions. Aspects of pulmonary health can be assessed from movement patterns of chest, nostrils and ribs.

A wide range of motion analysis systems allow movement to be captured in a variety of settings, which can broadly be categorized into direct (devices affixed to the body, e.g. accelerometry) and indirect (vision-based, e.g. video or optoelectronic) techniques. Direct methods allow kinematic information to be captured in diverse environments. For example, inertial sensors have been used as tools to provide insight into the execution of various movements (walking gait, discus, dressage and swimming). Sensor drift, which influences the accuracy of inertial sensor data, can be reduced during processing; however, this is yet to be fully resolved and capture periods remain limited. Additionally, it has been recognized that motion analysis systems for biomechanical applications should fulfil the following criteria: they should be capable of collecting accurate kinematic information, ideally in a timely manner, without encumbering the performer or influencing their natural movement. As such, indirect techniques can be distinguished as more appropriate in many settings compared with direct methods, as data are captured remotely from the participant imparting minimal interference to their movement. Indirect methods were also the only possible approach for biomechanical analyses previously conducted during sports competition. Over the past few decades, the indirect, vision-based methods available to biomechanists have dramatically progressed towards more accurate, automated systems. However, there is yet to be a tool developed which entirely satisfies the aforementioned important attributes of motion analysis systems. Thus, these analyses may be used in coaching and physical therapy in dancing, running, tennis, golf, archery, shooting biomechanics and other sporting and physical activities. Other uses include ergonomic training for occupations that subject persons to the dangers of repetitive stress disorders and other physical stressors related to motion and posture. The data can also be used in the design of furniture, self-training, tools, and equipment design.

The machine vision module may include one or more digital camera sensors for imaging one or more of pupil dilation, scleral erythema, changes in skin color, flushing, and/or erratic movements of a subject, for example. Other optical sensors may be used that operate with coherent light, or use a time of flight operation. In certain variants, the machine vision module comprises a 3D camera such Astra Embedded S by Orrbec.

### Thermal Sensor

In certain embodiments, the systems, devices and methods of the present technology include one or more thermal sensors including an infrared sensor, a thermometer, or the like. The thermal sensors may be incorporated in the wearable device or the remote device. The thermal sensor may be used to perform temperature measurements of one or more of a lacrimal lake and/or an exterior of tear ducts of the subject. The thermal sensor may be configured to detect temperature and temperature changes of air flow through the nose and/or the mouth of the subject. In some variations, the thermal sensors may comprise a thermopile on a gimbal, such as but not limited to a thermopile comprising an integrated infrared thermometer, 3V, single sensor (not array), gradient compensated, medical+−0.2 to +−0.3 degree kelvin/Centigrade, 5 degree viewing angle (Field of view—FOV).

### Photoacoustic Spectroscopy Vibrometry

In a separate sensor-fusion embodiment vibroacoustic, electric potential, photoacoustic/photothermal spectroscopy combined with an intensity-modulated quantum cascade laser (QCL), and a laser Doppler vibrometer (LDV) based on the Mach-Zehnder interferometer subsystems may be integrated and time-synchronized for non-contact detection of the biofield vibration signal resulting from the photocoustic/photothermal effect. The photo-vibrational spectrum obtained by scanning the QCL's wavelength in mid-Infra Red (MIR) range, coincides well with the corresponding spectrum obtained using typical FTIR equipment. The experiment demonstrated that the LDV is a capable sensor for applications in photoacoustic/photothermal spectroscopy, with potential to enable the detection of vital signs in open environment at safe standoff distance.

### Sensor Fusion

The fusion of data from any one or more sensors and using any combination of sensors can provide unique insights into subject's intracranial pressure, breath, facial micro-movement, nostril movement, heartbeat, blood flow, ventricular ejection fraction, and gut activity. Sensor and data fusion experiment results show that skin motion amplification detection efficiency, either with direct contact or through clothing, is better than that by short time Fourier transform and radar networking technology previously used in dynamic tracking and monitoring of the human body. For example, electric potential and vibroacoustic data within the broad frequency ranges described herein result in superbly accurate motion amplification so heart, lung and gut activity cycles can be detected from a distance.

### Foldable Sensor Device

In certain embodiments, the systems, devices and methods of the present technology include a sensor device including a plurality of sensors and having a foldable configuration (“foldable sensor device”) for forming a multi-layered structure from a planar structure (FIG. 5). In certain embodiments, the foldable sensor device includes a plurality of substrates for housing a plurality of sensors, the plurality of substrates being substantially co-planar in an unfolded configuration and stacked in a folded configuration. In the stacked configuration, the substrates may be substantially parallel or orthogonal to one another thereby defining a multi-layered structure. An example foldable sensor device has been described and illustrated in PCT/US21/63151 filed Dec. 13, 2021, the contents of which are herein incorporated by reference.

FIG. 5 illustrates a folded configuration of a foldable sensor device 500. In this folded configuration, a substrate 505 and substrate 515 may be stacked one above each other. In an unfolded configuration (not illustrated), the substrate 505, join member 512, substrate 513, join member 514, and/or substrate 515 are co-planar.

The arrangement of the sensors on the substrates 505, 513, and 515 may be such that in use, when the foldable sensor device 500 is positioned on or near the body of the subject, some of the sensors of the foldable sensor device 500 may face the user's body and/or some of the sensors of the foldable sensor device 500 may face outwardly towards the environment, away from the user's body. The sensors facing towards the user's body may capture physiological data of the user. The sensors facing away from the user's body may capture environmental data describing the environment surrounding the user. Both types of data, physiological and environmental, may be captured simultaneously by the foldable sensor device 500. The data capture may be continuous or intermittent.

In other embodiments (not shown), at least a portion of at least one sensor may be formed within the body of the substrate 505, substrate 513, and/or substrate 515. Such sensor or sensor portion may include filtering elements, such as a copper plate, light filter, and/or layer of piezoelectric material that reacts to being bent. The layer of piezoelectric material may function as a vibroacoustic sensor.

In certain embodiments, the sensors facing the user's body include a vibroacoustic sensor, a PPG/SpO2 sensor, and an electric potential sensor. The sensors facing away from the user's body include a pressure sensor, a temperature sensor, a humidity sensor, a light sensor, and an inertial measurement unit (IMU). In other embodiments, any other combination of sensors for detecting physiological and/or environmental signals may be used in the foldable sensor device 500. The types of sensors that can be used with the present technology is not particularly limited, and certain example sensors are described herein.

The substrate 505, join member 512, substrate 513, join member 514, and/or substrate 515 may include other electronic components, such as communication components including an antennae, power sources including a battery, storage devices including flash memory which may be removable, processors, a Universal Serial Bus (USB) port or other data transmission port, shielding components, grounding components and/or a signal amplifying component. One or more batteries may be included in the foldable sensor device 500. The batteries may be attached to the substrate 505, the substrate 513 and/or substrate 515. When the foldable sensor device 500 is folded, the batteries may be sandwiched between the substrate 505 and substrate 515. The batteries provide power to the sensors and/or other electronic components of the foldable sensor device 500. The antenna may be incorporated in the join members 512 and/or 514.

The foldable sensor device 500 may include a storage unit for storing data collected by the sensors. The storage unit may be communicatively coupled to the sensors to receive the data captured by the sensors. The storage unit may be accessed by a processor of the foldable sensor device 500. The data stored on the storage unit may be accessed via the USB port of the foldable sensor device 500 and/or via a wireless communication protocol, such as Wi-Fi or Bluetooth. The storage device may be removable, such as a removable flash memory device.

The foldable sensor device 500 may have various shapes beyond the illustrated embodiment. For example the foldable sensor device 500 may be derived from a polyhedron which is flattened (unfolded configuration) then folded (folded configuration). The arrangement of the sensors on the faces of the substrates may differ from that as illustrated.

There are a number of advantages with such a sensor-based foldable device, not least relating to manufacturing ease and improved functionality. With regard to manufacturing ease, sensors and other electronic components can be connected together on a planar configuration. Subsequent folding can create a stacked multi-layered configuration which has a smaller footprint than the unfolded configuration. Smaller footprints are advantageous for many uses, and particularly for wearable applications in which discreteness is preferred. Furthermore, such a multi-layered sensor device can be useful for positioning sensors on different planes thereof and therefore at different proximities to a target. Additionally, sensors can be pointed in different directions. For example, sensors for detecting signals associated with a subject wearing the device may be pointed towards the subject and closer to the subject and sensors for detecting environmental parameters may be pointed towards the environment (away from the subject) and further from the subject. The sensors may include an acoustic sensor and/or an electric potential sensor or a contextual sensor for detecting signals from an environment of the subject.

In certain embodiments, the foldable sensor device comprises: a first substrate having a first sensor; a second substrate having a second sensor; and a first join member connecting the first substrate and the second substrate such that the first substrate and the second substrate are foldable relative to each other to form a folded configuration having multiple layers with the first substrate stacked relative to the second substrate. The first sensor may be positioned on a first surface of the first substrate and the second surface may be positioned on a second surface of the second substrate, the first surface and the second surface being co-planar when in an unfolded configuration and stacked one above each other when in a folded configuration, with the first surface facing away from the second surface. The first sensor may be configured to detect signals from a user of the foldable sensor device and the second sensor may be configured to detect signals from an environment of the user, and wherein the first sensor faces away from the second sensor.

The foldable sensor may include an enclosure housing the first substrate, the second substrate and the first join member when the first substrate and the second substrate are in the folded configuration. There may also be provided a retaining member for retaining the first substrate and the second substrate in the folded configuration. The enclosure may have a configuration which is wearable by the user against or proximate a body part of the user and which is selected from one or more of: a strap, a band aid, a patch, a watch, a bandage, an item of jewelry, a head piece, an eye piece, an ear piece, a mouth piece, a collar, an item of clothing, a belt, a support, bedding, a blanket, a pillow, a cushion, a support surface of a seat, and a head-rest.

The first and second sensors may comprise any of the sensors described herein. In certain embodiments, the first sensor comprises one or more of a vibroacoustic sensor, a PPG/SpO2 sensor, and an electric potential sensor. The second sensor may comprise one or more of a pressure sensor, a temperature sensor, a humidity sensor, a light sensor, and an IMU.

One or both of the first sensor and the second sensor may be configured to be communicatively connected to a processor of the foldable sensor device and/or a remote processor. The processor is configured to trigger, based on a data collection protocol, one or both of the first sensor and the second sensor to one or more of: start collecting data, stop collecting data, start storing the collected data and stop storing the collected data. The trigger event may comprise one or more of an intensity of a detected activity, an intensity of a detected signal compared to a threshold intensity, and a frequency of a detected signal compared to a threshold frequency.

The first sensor and the second sensor are connected to a power source and wherein the data collection protocol is based on a consideration of balancing battery life with collection of pertinent data or storage of pertinent data. The data collection protocol may be based on a predetermined time interval and/or a trigger event.

The vibroacoustic sensor has a vibroacoustic sensor sampling rate for capturing the vibroacoustic signals and the electric potential sensor has an electric potential sensor sampling rate for capturing the electric potential signals, each of the vibroacoustic sensor sampling rate and the electric potential sensor sampling rate being determined to optimize a battery life of the respective vibroacoustic sensor and the electric potential sensor.

### Devices

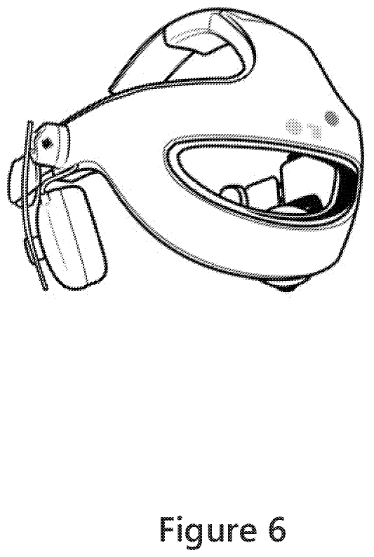

Any one or more of the sensors described herein, and optionally any portion of the computer system can be embodied in a device having a suitable configuration for an intended use. Referring to FIGS. 6-21, various embodiments of wearable devices of embodiments of the present technology are illustrated.

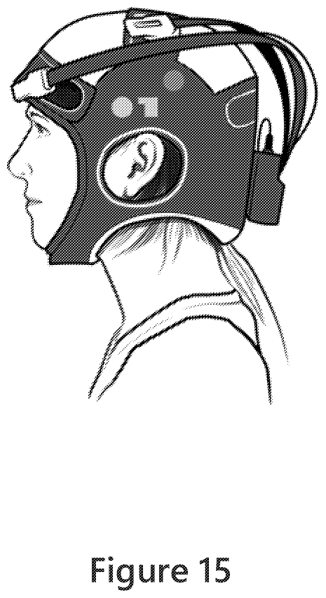

In certain embodiments, the wearable device is configured as a head piece which may cover the subject's head like a helmet (FIG. 15).

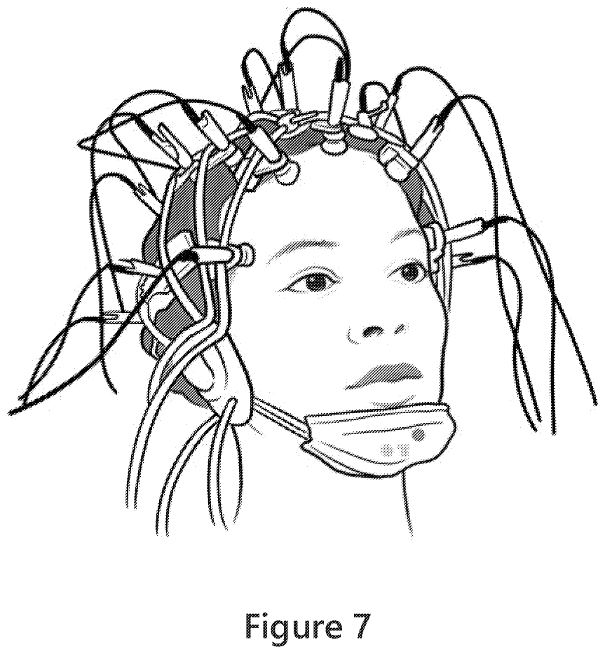

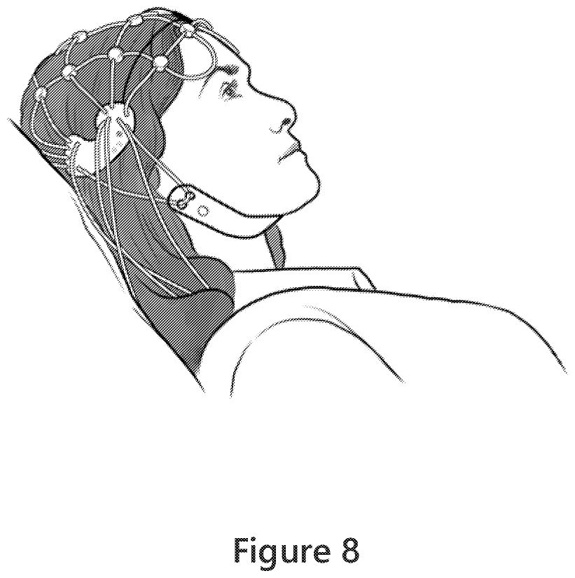

In certain embodiments, the wearable device is configured as a head piece which contacts or is configured to be positioned proximate only a portion of the subject's head. Such wearable devices may comprises discrete sensor modules, including one or more sensors, which are spaced apart and configured to be positioned at different locations on the subject's head. One or more straps may be provided for supporting the sensor modules on the subject's head and/or for interconnecting the sensor modules (FIGS. 7 and 8).

Alternatively, the different sensor modules or sensors may be housed in one enclosure which is configured to extend over a portion of the subject's head (FIGS. 6, 10, 11, 14).

In particular, sensors may be positioned so that, in use, they rest proximate one or more of the base of the skull, behind the ears, and the temples of the subject.

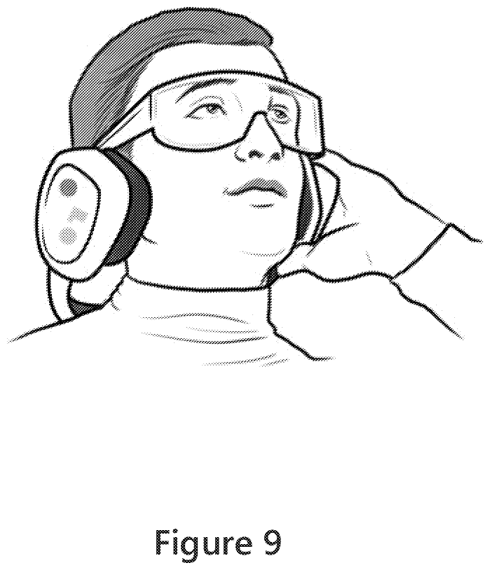

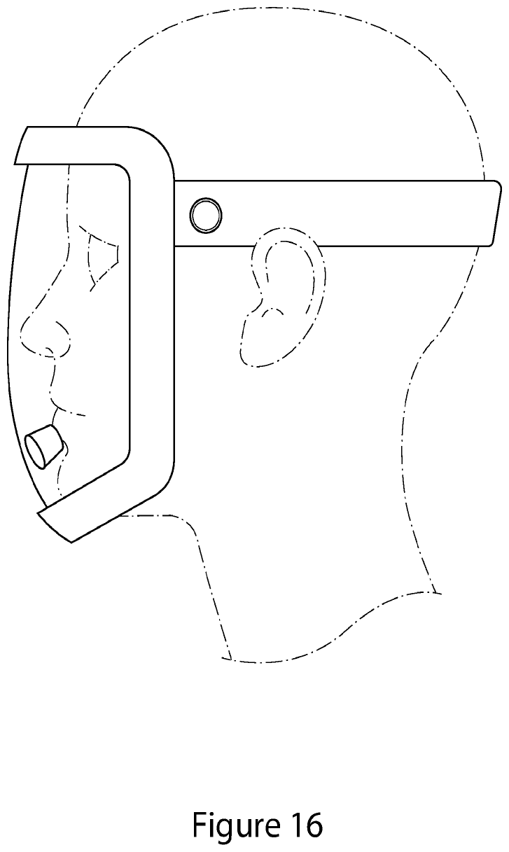

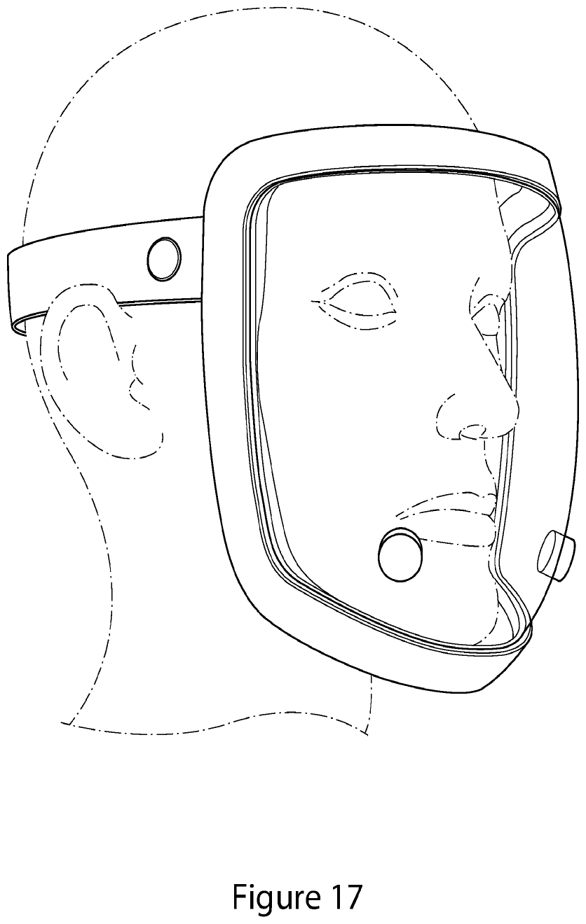

In certain embodiments, the wearable device is configured as an eye piece which may cover one or both of the subject's eyes (FIGS. 9, 16 and 17). The eye piece may be configured as glasses or as a full face mask (FIGS. 16 and 17). An example of a mask that can be used in the present technology is described in PCT/US21/63152 filed Dec. 13, 2021, the contents of which are herein incorporated by reference.

In certain embodiments, a Virtual Reality or an Augmented Reality head-set is also provided.

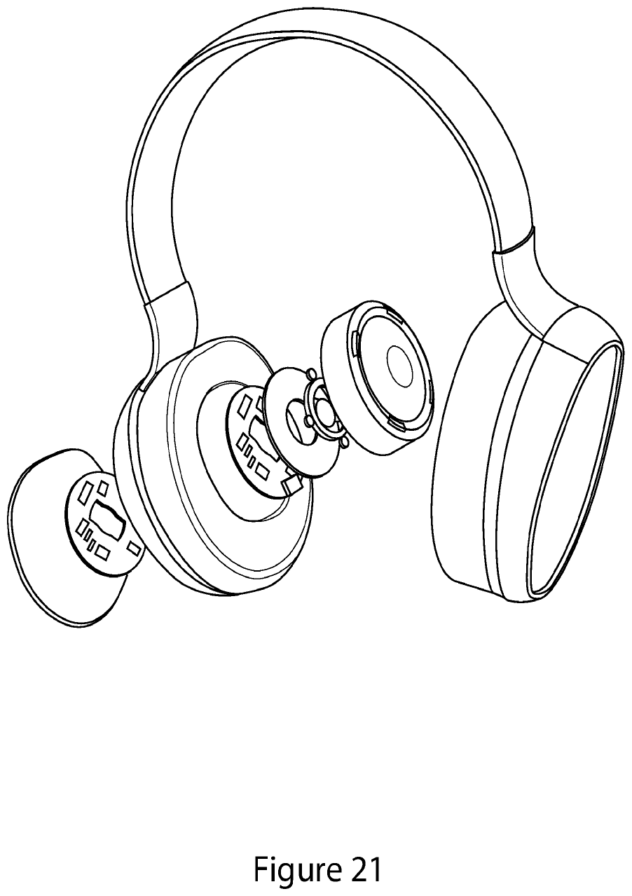

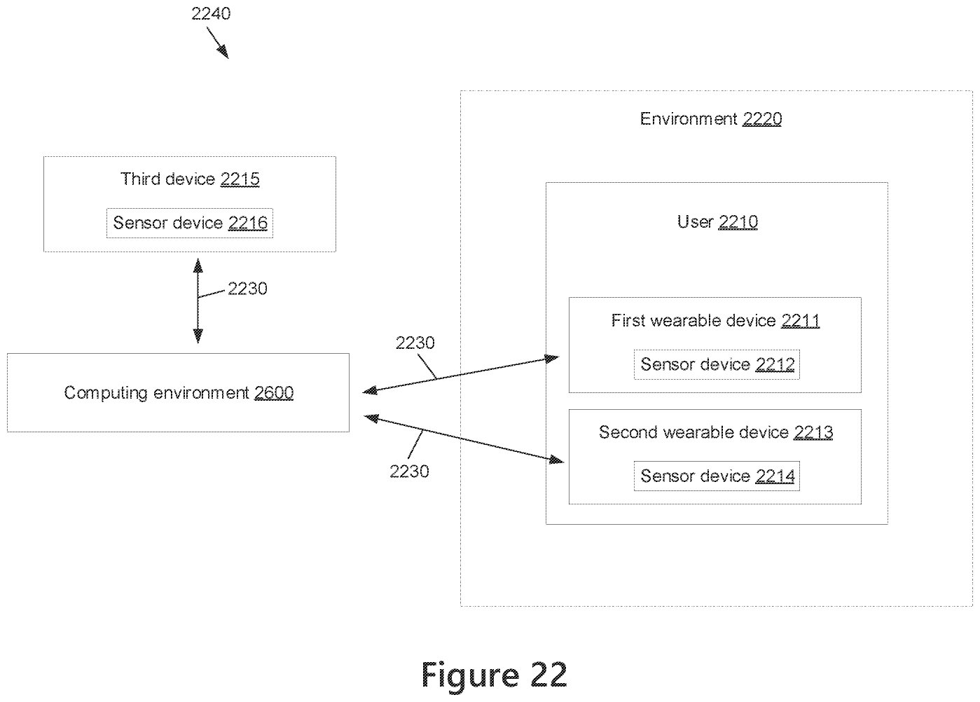

In certain embodiments, the wearable device is configured as an earpiece which may cover, or be at least partially insertable in, one or both of the subject's ears (FIGS. 6, 7, 9, 10, 11, 12, 13, 20 and 21). FIG. 21, for example, illustrates the wearable device as headphones including left and right ear portions and a connecting strap. A voice coil vibroacoustic sensor is included in at least one of the ear portions of the headphones. The headphones may also include a speaker, separated from the vibroacoustic transducer by a dampener to avoid signal interference. The speaker may be used to provide sound or haptic stimulation to the subject.

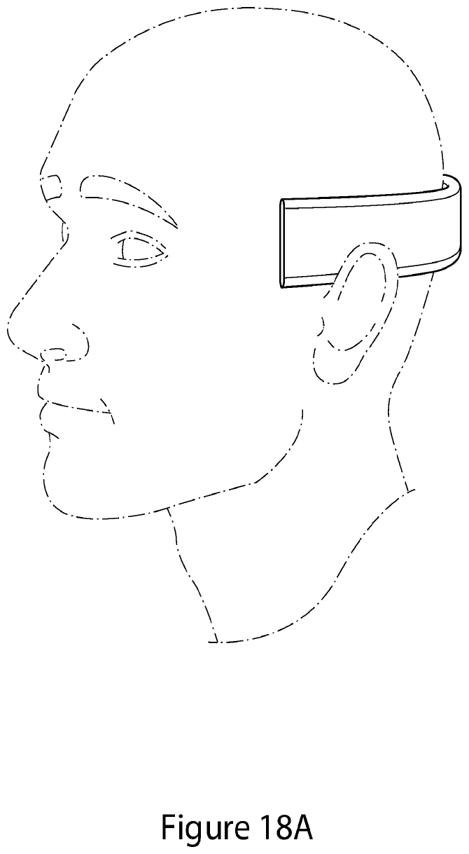

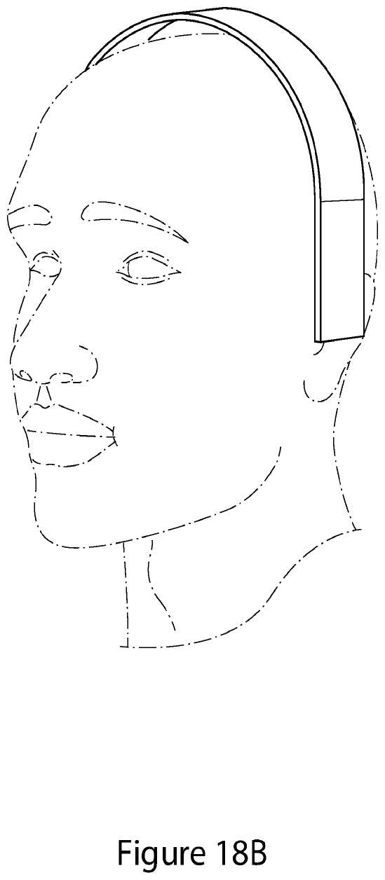

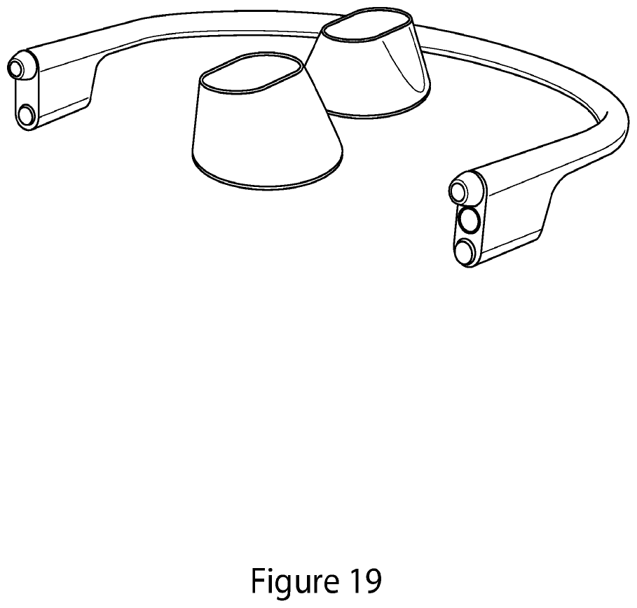

In certain embodiments, the wearable device is configured as a head band incorporated one or more of the sensors (FIGS. 18A, 18B and 19) which can be worn over the head and either cover, or not, the ears.

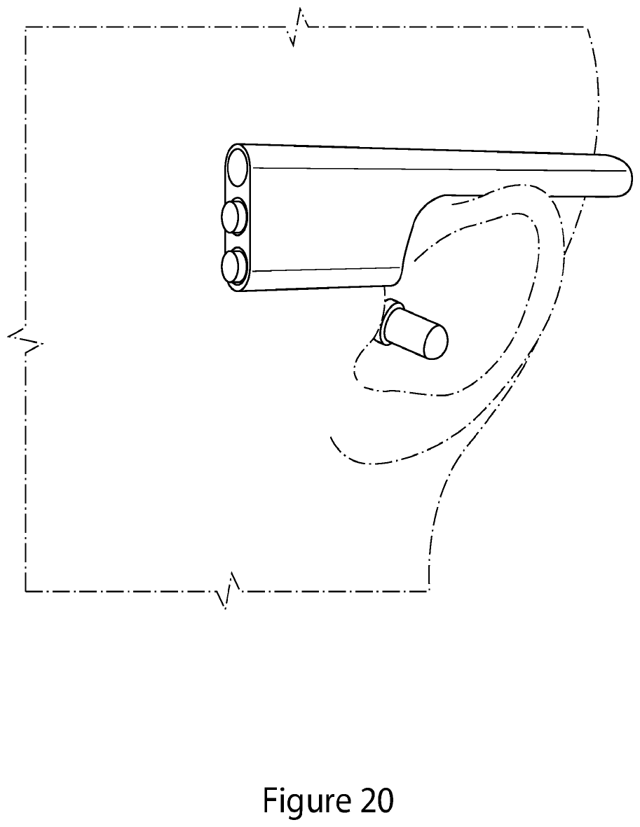

The wearable device may comprise one piece or more than one piece. For example, in FIG. 20, the wearable device comprises a head band portion configured to extend around a back portion of the head and an ear pod portion configured to be inserted in the ear.

In certain embodiments, the wearable device of FIG. 7 is configured to capture anechoic chamber activity and pressure change localization, as an example.

In certain embodiments, the wearable device of FIG. 9 is configured to measure cerebral metabolic oxygen utilization and auto regulation using AV/AR stimulation, as an example.

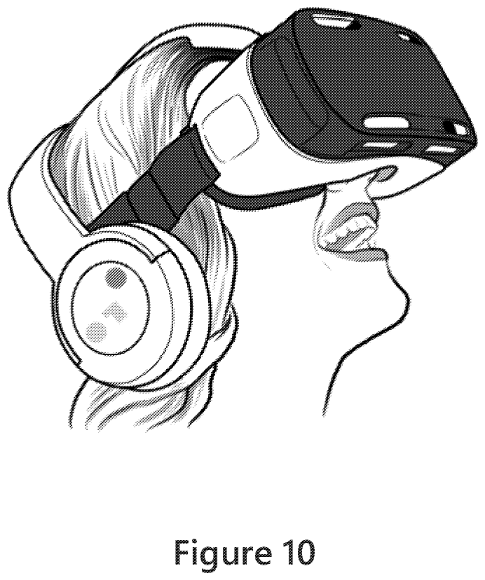

In certain embodiments, the wearable device of FIG. 10 is configured to provide AR/VR stimulation and measure anechoic chamber activity, as an example.

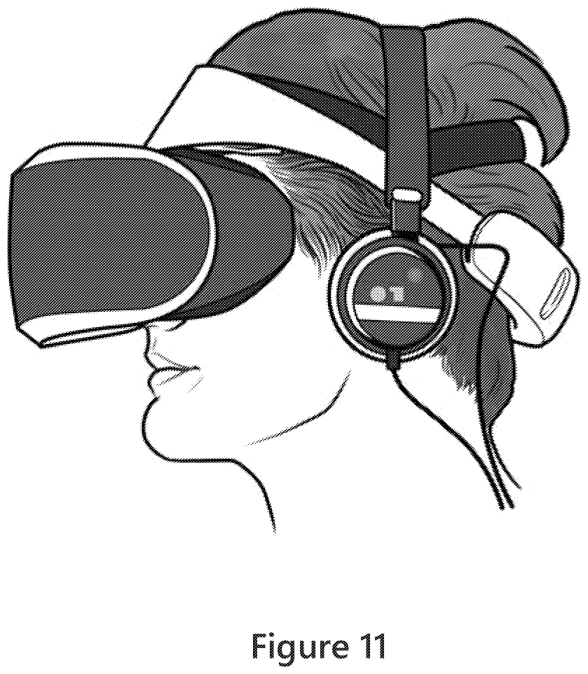

In certain embodiments, the wearable device of FIG. 11 is configured to provide AR/VR stimulation and have sensors positioned at a base of skull, as an example.

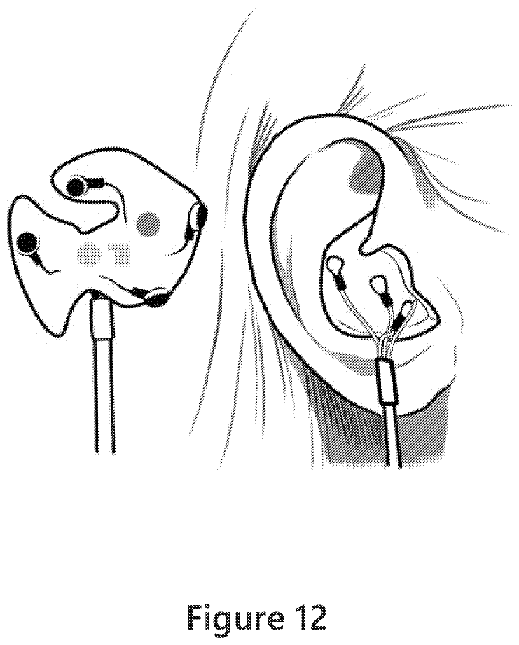

In certain embodiments, the wearable device of FIG. 12 is configured as an earbud and includes sensors configured to measure cerebral blood volume due to cerebral vasoconstriction or dilatation or the pressure-volume index to determine alterations in transmural pressure as optimally attenuated by cerebral arteriolar vasoconstriction as affected by autoregulatory status.

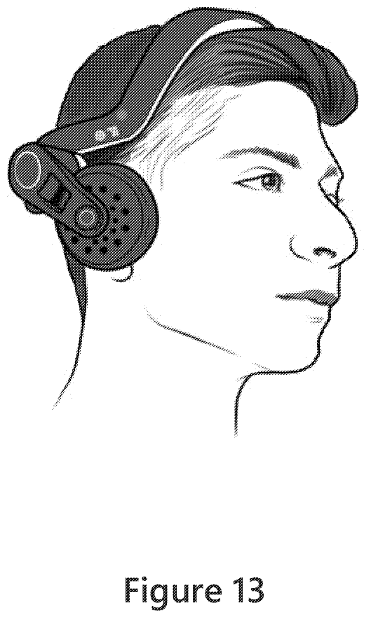

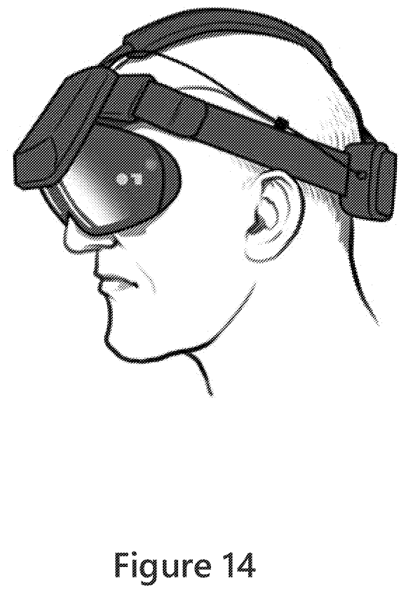

In certain embodiments, the wearable devices of FIGS. 13 and 14 have adjustable portions for adjusting a positioning of the sensors contained therein.

### Stimulator

As well as sensors, the devices and methods of the present technology may include one or more stimulator modules or devices for providing a signal to the subject. The stimulator is any sensor or device that can emit a signal that can stimulate the subject. The stimulator device or the stimulator module may be incorporated in the wearable device including the sensors, or be separate therefrom. The system of the present technology may be configured to determine, optimize and/or tune the signal to be applied to the subject based at least in part on the collected data.

In certain embodiments, the stimulator is an AR/VR module which can provide image data as stimulation and include an AR/VR head set or goggles (for example FIGS. 10, 11 and 14).

In certain embodiments, the stimulator is a speaker, such as a speaker included in a wearable device with a headphones configuration (for example FIGS. 9, 10, 11, 12, 13, 20, and 21).

The stimulator may comprise any of these driver technologies, or a combination thereof: dynamic or moving coil, balanced armature, planar magnetic, and electrostatic, magnetostriction/bone conduction.

The driver or drivers may be configured as in the ear, circum aural or supra aural device depending on the intended use.

In certain embodiments, the stimulator is a tablet comprising a display and a speaker for emitting visual and acoustic signals, respectively, to the subject. The tablet may further include a camera.

### Methods

### Method—Monitoring Intracranial Pressure

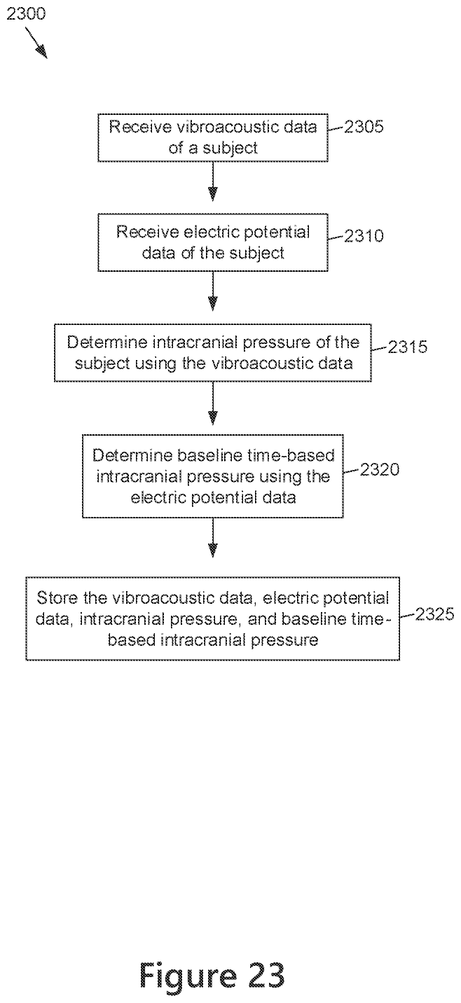

FIG. 23 is a flow diagram of a method 2300 for monitoring intracranial pressure in accordance with various embodiments of the present technology. In one or more aspects, the method 2300 or one or more steps thereof may be performed by a computing system, such as the computing environment 2600. All or a portion of the steps may be executed by any of the devices described herein. The method 2300 or one or more steps thereof may be embodied in computer-executable instructions that are stored in a computer-readable medium, such as a non-transitory mass storage device, loaded into memory and executed by a CPU. Some steps or portions of steps in the flow diagram may be omitted, changed in order, and/or executed in parallel.

At step 2305, vibroacoustic data of the subject may be received. The vibroacoustic data may have been measured by one or more vibroacoustic sensors. The vibroacoustic sensors may be placed on different locations on the head of the subject. The vibroacoustic sensors may include voice coil sensors.

The vibroacoustic data may be collected by sensors in a wearable device worn by the subject, such as a head-worn device. The wearable device may include one or more stimulators that output vibroacoustic signals. The wearable device may be an earpiece placed in the subject's ear or ears and/or over the subject's ear or ears. The earpiece may include a voice coil sensor and/or a speaker. The speaker may be separated from the voice coil sensor by a dampener. The vibroacoustic sensors may be placed against the subject's skin. The vibroacoustic data may include vibroacoustic signals within a bandwidth ranging from about 0.01 Hz to about 160 kHz, or about 0.01 Hz to about 20 kHz.

At step 2310, electric potential data of the subject may be collected. The electric potential data may have been measured by one or more electric potential sensors. The electric potential data may have been measured by a sensor integrated in a wearable device, such as the devices described above at step 2510 for capturing vibroacoustic data. The wearable device may include both the electric potential sensors and the vibroacoustic sensors. The electric potential data may be captured using a patch, which may be placed against the subject's neck. The patch may include electric potential sensors and/or vibroacoustic sensors. The vibroacoustic data and electric potential data may be collected during a same time period or different time periods. The vibroacoustic data and electric potential data may be collected simultaneously and be time-locked.

The electric potential sensor may be co-located with the vibroacoustic sensor. Alternatively, the electric potential sensor may be positioned on the subject but not in the wearable device. The electric potential sensor may be included in another wearable device, such as a patch. The electric potential sensor may be positioned remote from the subject and configured to detect the electric potential signals remotely. The electric potential data and/or vibroacoustic data may be collected non-invasively, such as by external sensors worn by the subject. The sensors and/or a wearable device containing the sensors may be non-invasively coupled to the subject's head.

The vibroacoustic data, electric potential data, and/or any other collected data may be time-stamped to indicate a time at which the vibroacoustic data and/or electric potential data was collected. The vibroacoustic data and/or electric potential data may be collected over a pre-determined length of time, such as ten seconds. The vibroacoustic data and/or electric potential data may be collected over a pre-determined number of heart cycles of the subject, such as over one hundred heart cycles. The vibroacoustic data and/or electric potential data may include data collected at multiple different non-contiguous time periods.

The vibroacoustic data, electric potential data, and/or any other collected data may be recorded at a pre-determined sampling rate. The sampling rate for the vibroacoustic data, electric potential data, and/or any other collected data may be a same sampling rate or a different sampling rate. The sampling rate may be selected to optimize a battery life of the vibroacoustic sensors, electric potential sensors, and/or wearable device containing the sensors. The sampling rate for the vibroacoustic sensor and/or electric potential sensor may be switched between a relatively high sampling rate and a relatively low sampling rate to optimize data resolution and/or optimize battery life.

At step 2315, an intracranial pressure of the subject may be determined. The intracranial pressure may be determined using the vibroacoustic data and/or the electric potential data. The intracranial pressure may include multiple components including an intracranial pressure component related to baseline time-based intracranial events of the subject caused by heartbeats and/or breaths of the subject. As the subject breaths and/or their heart beats, there will be pulsatile changes in the intracranial pressure component. The intracranial pressure component related to the subject's breath and/or heartbeat may therefore comprise pulsatile intracranial pressure gradients. The intracranial pressure may also include an intracranial pressure component which is not related to the breath and/or heartbeat but may be related to a condition of the subject or to a contextual event.

At step 2320, the electric potential data may be used to identify the baseline time-based intracranial events, i.e. a time signature of the heartbeat and/or the breath. As the electric potential data and the vibroacoustic data are time-locked, the corresponding vibroacoustic data corresponding to the heartbeat and/or the breath may be identified. The component of the vibroacoustic data corresponding to the heartbeat and/or breath may be separated from the vibroacoustic data. At least a portion of the remaining component of the vibroacoustic data may therefore be used to identify the component of the vibroacoustic data corresponding to the condition of the subject or the contextual event.

Baseline time-based events of the subject may be determined. Portions of the vibroacoustic data corresponding to those baseline time-based events may be determined. An occurrence of a change in the intracranial pressure due to a condition not related to the baseline time-based events may be determined by identifying portions of the vibroacoustic data not related to the baseline time-based events.

A change in the intracranial pressure of the subject may be determined. The change may be determined based on the vibroacoustic data and/or electric potential data. The change may be determined relative to the intracranial pressure related to the heartbeat and/or the breath. A rate of change of intracranial pressure may be determined.

The intracranial pressure changes may be detected by the electric potential sensor by disambiguating the base intracranial pressure gradients from the electric potential data or the vibroacoustic data. An occurrence of a time-based change in intracranial pressure may be determined by comparing a magnitude of the intracranial pressure to a threshold magnitude. The threshold may have been predetermined based on the base intracranial pressure.

Changes to intracranial pressure may be related to a condition of the subject or may be contextual related. The time-based changes to intracranial pressure can be compared to biomarkers of various conditions to identify if the subject has an onset of a given condition, a precursor to a given condition, and/or an increase/decrease in the condition. The condition can be an event such as a fall or an impact of the subject. The condition can be the presence or absence of a disease. The condition can be a progression of a disease state such as a tumor, a disease, etc.

A magnitude, a frequency pattern and/or an aperiodic pattern of the intracranial pressure changes may be quantified to determine a condition of the subject. The onset of the condition may be determined by comparing a magnitude of the detected time-based change in the intracranial pressure to a threshold magnitude. For contextual-related intracranial pressure changes (such as atmospheric conditions, external events, etc.) those time-based changes can then be compared to biomarkers.

A trained machine learning algorithm (MLA) may be applied to the vibroacoustic data and/or electric potential data. Additional collected data may be input to the MLA, such as temperature data of the subject, movement data of a body part of the subject, and/or volatile organic compound data of the subject. The temperature data may include temperature, or changes in temperature, of air flowing through a nose or a mouth of the subject.

At step 2325 the collected and determined data may be stored. The vibroacoustic data, electric potential data, base intracranial pressure, and/or changes to the intracranial pressure may be stored. All or a portion of this data may be stored in a database. The device or devices that collected the data may transmit it to a server. The data may be transmitted to the server using a communication module of the device. The server and/or a database may receive and store the data. The server may perform some of the steps described above, such as determine the base intracranial pressure and/or determining changes to the intracranial pressure.

Method—Receiving and Storing Detected Data from a Subject

FIG. 24 is a flow diagram of a method 2400 for applying vibroacoustic signals or acoustic signals and recording a response in accordance with various embodiments of the present technology. In one or more aspects, the method 2400 or one or more steps thereof may be performed by a computing system, such as the computing environment 2600. All or a portion of the steps may be executed by any of the devices described herein. The method 2400 or one or more steps thereof may be embodied in computer-executable instructions that are stored in a computer-readable medium, such as a non-transitory mass storage device, loaded into memory and executed by a CPU. Some steps or portions of steps in the flow diagram may be omitted, changed in order, and/or executed in parallel.

At step 2405, vibroacoustic signals may be applied to a subject. The vibroacoustic signals may be applied by one or more stimulators. The stimulators may be in a wearable device worn by the subject, such as a wearable device worn on a head, face, body, and/or neck of the subject. The wearable device may include one or more speakers configured to emit a signal. The speakers may be housed in an ear piece of the wearable device. The speakers may be separated from voice coil sensors of the wearable device by a dampener.

Multiple vibroacoustic signals may be output by the stimulators. The vibroacoustic signals may have various frequencies, intensities, durations, and/or directions. The vibroacoustic signals may include a sweep-frequency stimulation with a bandwidth of about 0.01 Hz to 80 kHz. The vibroacoustic signals may be a predetermined vibroacoustic signal pattern retrieved from a sound library.

The vibroacoustic signals may be binaural audio. The vibroacoustic signals may be retrieved from a binaural sound library containing multiple binaural sounds. The binaural audio may include a lower frequency signal and/or a higher frequency signal. The lower frequency signal and the higher frequency signal may be alternatingly applied to the subject's right and left ear. The frequency of the alternation between the respective signals being applied to the left and right ears being may be from about 0.001 Hz to 0.005 Hz, about 0.005 to 0.01 Hz, about 0.01 Hz to 0.05 Hz, about 0.05 Hz to 0.1 Hz, about 0.1 Hz to 0.5 Hz, about 0.5 Hz to 1 Hz, about 1 Hz to 5 Hz, about 5 Hz to 50 Hz, about 50 Hz to 200 Hz, about 200 Hz to 500 Hz, or about 500 Hz to 1000 Hz.

In addition to or instead of the vibroacoustic signals, sound signals, haptic signals, and/or visual signals may be applied to the subject. The vibroacoustic signals, sound signals, haptic signals, and/or visual signals may be emitted by a remote device that is remote from the subject. The remote device may include one or more electric potential sensors, which may be used to collect electric potential data of the subject.

At step 2410, vibroacoustic data of the subject may be received. The vibroacoustic data may have been measured by one or more vibroacoustic sensors. The vibroacoustic sensors may be placed on different locations on the head of the subject. The vibroacoustic sensors may include voice coil sensors. The vibroacoustic data may be responsive to the signals applied to the subject.

The vibroacoustic data may be collected by sensors in a wearable device worn by the subject, such as a head-worn device. The wearable device may include the stimulators that output the vibroacoustic signals at step 2405. The device may be an earpiece placed in the subject's ear or ears and/or over the subject's ear or ears. The earpiece may include a voice coil sensor and/or a speaker. The speaker may be separated from the voice coil sensor by a dampener. The vibroacoustic sensors may be placed against the subject's skin. The vibroacoustic data may include vibroacoustic signals within a bandwidth ranging from about 0.01 Hz to about 160 kHz.

The wearable device may comprise two earpieces. Each of the earpieces may be positionable in or over a respective ear of the subject. The vibroacoustic sensor may comprise at least one voice coil sensor in each of the earpieces. Because two earpieces on opposite sides of the individual's head are being used to collect vibroacoustic data, the vibroacoustic signals detected in each earpiece can be used to identify differences associated with left and right brain hemispheres of the subject. One of the earpieces may comprise a voice coil sensor, and the other earpiece may comprise a speaker configured to emit the vibroacoustic signals.

The vibroacoustic data may be collected by one or more patches that are non-invasively coupled to the subject's skin. The patches may include one or more electric potential sensors and/or one or more vibroacoustic sensors.

At step 2415, electric potential data of the subject may be collected. The electric potential data may have been measured by one or more electric potential sensors. The electric potential data may have been measured by a sensor integrated in a wearable device, such as the devices described above at step 2405 for capturing vibroacoustic data. The wearable device may include both the electric potential sensors and the vibroacoustic sensors. The electric potential data may be captured using a patch, which may be placed against the subject's neck. The patch may include electric potential sensors and/or vibroacoustic sensors. The vibroacoustic data and electric potential data may be collected during a same time period or different time periods. The electric potential data may be responsive to the signals applied to the subject.

The vibroacoustic data, electric potential data, and/or any other collected data may be time-stamped to indicate a time at which the vibroacoustic data and/or electric potential data was collected. The vibroacoustic data and/or electric potential data may be collected over a pre-determined length of time, such as ten seconds. The vibroacoustic data and/or electric potential data may be collected over a pre-determined number of heart cycles of the subject, such as over one hundred heart cycles. The vibroacoustic data and/or electric potential data may include data collected at multiple different non-contiguous time periods.

The vibroacoustic data, electric potential data, and/or any other collected data may be recorded at a pre-determined sampling rate. The sampling rate for the vibroacoustic data, electric potential data, and/or any other collected data may be a same sampling rate or a different sampling rate. The sampling rate may be selected to optimize a battery life of the vibroacoustic sensors, electric potential sensors, and/or wearable device containing the sensors. The sampling rate for the vibroacoustic sensor and/or electric potential sensor may be switched between a relatively high sampling rate and a relatively low sampling rate to optimize data resolution and/or optimize battery life.

At step 2420 the vibroacoustic data and/or electric potential data may be stored. Information regarding the vibroacoustic signals applied at step 2405 may also be stored and associated with the collected vibroacoustic data and/or electric potential data. The data may be stored in a database. The device that collected the data may transmit it to a server or other device for storage and/or analysis. The data may be transmitted using a communication module of the device. A server and/or database may receive and store the data.

### Method—Determining an Intracranial Pressure of a Subject

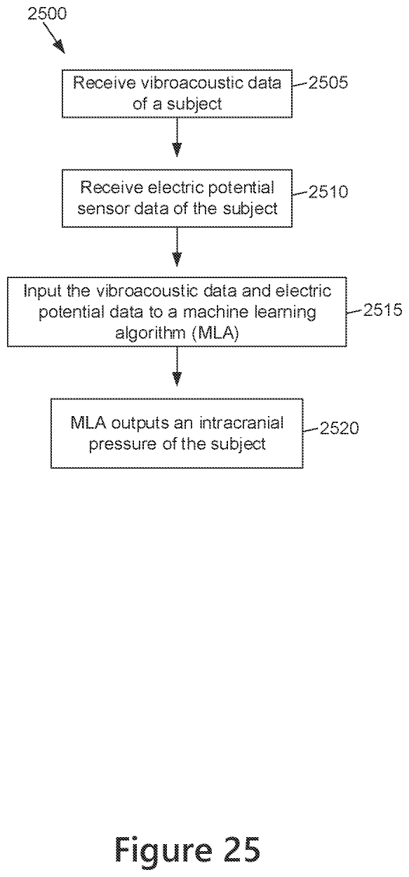

FIG. 25 is a flow diagram of a method 2500 for determining an intracranial pressure in accordance with various embodiments of the present technology. In one or more aspects, the method 2500 or one or more steps thereof may be performed by a computing system, such as the computing environment 2600. All or a portion of the steps may be executed by any of the devices described herein. The method 2500 or one or more steps thereof may be embodied in computer-executable instructions that are stored in a computer-readable medium, such as a non-transitory mass storage device, loaded into memory and executed by a CPU. Some steps or portions of steps in the flow diagram may be omitted, changed in order, and/or executed in parallel.

At step 2505, vibroacoustic data may be received. At step 2510, electric potential sensor data may be received. Actions performed at steps 2505 and 2510 may be similar to those described above with regard to steps 2305 and 2310 of the method 2300. Rather than receiving data at steps 2505 and 2510, the data may be retrieved, such as from a database and/or from a wearable device.

Additional data related to the subject may be received, such as temperature data of the subject, movement data of a body part of the subject, and/or a volatile organic compound from the subject.

At step 2515, the vibroacoustic data, electric potential data, and/or any additional data related to the subject may be input to a machine learning algorithm (MLA). The MLA may have been trained to use the vibroacoustic data and/or electric potential data to predict an intracranial pressure of a subject. In order to train the MLA, a labelled data set may have been developed. The labelled data set may include multiple data points, where each data point includes vibroacoustic data and/or electric potential data of a subject and a corresponding label. The label may include an intracranial pressure of the subject. The intracranial pressure in the label may be a measured intracranial pressure and/or an estimate intracranial pressure. Using the label, the MLA may be trained to predict intracranial pressure of a subject based on vibroacoustic data and/or electric potential data.

The MLA may have been trained using a high dimensional dissimilarity matrix. A high dimensional dissimilarity matrix is an efficient method to evaluate dissimilarity between any number of multi-dimensional distributions in some representational feature space where a distance measure between any number single features, the ground distance, can be explicitly calculated. The dissimilarity matrix summarizes this multitude of distances from individual features to full distributions.

At step 2520 the MLA may output the intracranial pressure of the subject. The outputted intracranial pressure may be a predicted intracranial pressure. The output may be displayed to a user, such as a health care provider for the subject.

### Computing Environment

FIG. 26 illustrates an embodiment of the computing environment 2600. In some embodiments, the computing environment 2600 may be implemented by any of a conventional personal computer, a network device and/or an electronic device (such as, but not limited to, a mobile device, a tablet device, a server, a controller unit, a control device, etc.), and/or any combination thereof appropriate to the relevant task at hand. In some embodiments, the computing environment 2600 comprises various hardware components including one or more single or multi-core processors collectively represented by processor 2610, a solid-state drive 2620, a random access memory 2630, and an input/output interface 2650. The computing environment 2600 may be a computer specifically designed to operate a machine learning algorithm (MLA). The computing environment 2600 may be a generic computer system.

In some embodiments, the computing environment 2600 may also be a subsystem of one of the above-listed systems. In some other embodiments, the computing environment 2600 may be an “off-the-shelf” generic computer system. In some embodiments, the computing environment 2600 may also be distributed amongst multiple systems. The computing environment 2600 may also be specifically dedicated to the implementation of the present technology. As a person in the art of the present technology may appreciate, multiple variations as to how the computing environment 2600 is implemented may be envisioned without departing from the scope of the present technology.

Those skilled in the art will appreciate that processor 2610 is generally representative of a processing capability. In some embodiments, in place of or in addition to one or more conventional Central Processing Units (CPUs), one or more specialized processing cores may be provided. For example, one or more Graphic Processing Units 2611 (GPUs), Tensor Processing Units (TPUs), and/or other so-called accelerated processors (or processing accelerators) may be provided in addition to or in place of one or more CPUs.

System memory will typically include random access memory 2630, but is more generally intended to encompass any type of non-transitory system memory such as static random access memory (SRAM), dynamic random access memory (DRAM), synchronous DRAM (SDRAM), read-only memory (ROM), or a combination thereof. Solid-state drive 2620 is shown as an example of a mass storage device, but more generally such mass storage may comprise any type of non-transitory storage device configured to store data, programs, and other information, and to make the data, programs, and other information accessible via a system bus 2660. For example, mass storage may comprise one or more of a solid state drive, hard disk drive, a magnetic disk drive, and/or an optical disk drive.

Communication between the various components of the computing environment 2600 may be enabled by a system bus 2660 comprising one or more internal and/or external buses (e.g., a PCI bus, universal serial bus, IEEE 1394 “Firewire” bus, SCSI bus, Serial-ATA bus, ARINC bus, etc.), to which the various hardware components are electronically coupled.

The input/output interface 2650 may allow enabling networking capabilities such as wired or wireless access. As an example, the input/output interface 2650 may comprise a networking interface such as, but not limited to, a network port, a network socket, a network interface controller and the like. Multiple examples of how the networking interface may be implemented will become apparent to the person skilled in the art of the present technology. For example the networking interface may implement specific physical layer and data link layer standards such as Ethernet, Fibre Channel, Wi-Fi, Token Ring or Serial communication protocols. The specific physical layer and the data link layer may provide a base for a full network protocol stack, allowing communication among small groups of computers on the same local area network (LAN) and large-scale network communications through routable protocols, such as Internet Protocol (IP).

The input/output interface 2650 may be coupled to a touchscreen 2690 and/or to the system bus 2660. The touchscreen 2690 may be part of the display. In some embodiments, the touchscreen 2690 is the display. The touchscreen 2690 may equally be referred to as a screen 2690. In the embodiments illustrated in FIG. 26, the touchscreen 2690 comprises touch hardware 2694 (e.g., pressure-sensitive cells embedded in a layer of a display allowing detection of a physical interaction between a user and the display) and a touch input/output controller 2692 allowing communication with the display interface 2640 and/or the system bus 2660. The display interface 2640 may include and/or be in communication with any type and/or number of displays. In some embodiments, the input/output interface 2650 may be connected to a keyboard (not shown), a mouse (not shown) or a trackpad (not shown) allowing the user to interact with the computing environment 2600 in addition to or instead of the touchscreen 2690.

According to some embodiments of the present technology, the solid-state drive 2620 stores program instructions suitable for being loaded into the random access memory 2630 and executed by the processor 2610 for executing acts of one or more methods described herein. For example, at least some of the program instructions may be part of a library or an application. Some or all of the components of the computing environment 2600 may be integrated in a multi-layer sensor device and/or in communication with the multi-layer sensor device. The processor may be configured to process the data obtained by the multi-layer sensor device, and provide an output, such as to a smartphone of an operator of the system.

### Uses—Operation

The devices, systems and methods of the present technology, in certain embodiments, harvest brain and skull passive vibroacoustics, active vibrometry, pressure fluctuations, and electric potentials in order to analyze the connectivity pattern of parts of the brain sensitive to sound with other non-auditory parts of the brain—parts of the brain responsible for speech, attention, learning, or fear, for example. Aspects and embodiments of the present system support real-time bio-feedback and can also compile a personalized library of audible and inaudible sounds that evoke specific biophysical responses with predictable health benefits.

The devices, systems and methods of the present technology, in certain embodiments, can harvest information discarded and/or ignored by current instrumentation as “noise” and can match individual skull/brain resonance frequencies in biofeedback experiments to tune and target audible and inaudible soundscapes on anxiety and depression, in cancer patients for example, and comfort patients, such as critically ill infants in intensive care units. Our algorithms can help individuals learn foreign languages easier, enjoy a wider variety of music by making their less preferred bright instruments fade into the mix, and fully experience the audible and inaudible soundscape around them.

The devices, methods and systems of the present technology can be integrated into non-contact, smart alarm solutions for screening and diagnosing pre-symptomatic and asymptomatic infectious diseases like COVID-19, influenza, and tuberculosis (TB); as well as high burden and high mortality diseases like carotid artery and coronary artery disease, and heart failure. This ability is enabled by tuning into data and information residing in what is traditionally thought of as biological “noise” and having for example, low frequency, and low amplitude.

The devices, methods and systems of the present technology, in certain embodiments, are able to accomplish non-contact diagnosis of infectious diseases and artery and heart disease by taking on the concept of GRAY data full on. Less than 10% of data generated by the heart, lung, gut and other tissues is available for decision-making at the bedside because the majority of gray and white data, together characterized as “noise” lies below or above the human ear's perception. The GRAY data, as referred to herein, are “known unknown” low frequency, low amplitude data and the WHITESPACE data, as referred to herein, are “unknown unknown” biological data.

The devices, methods and systems of the present technology, in certain embodiments, fuse data from an electric potential sensor that quantifies tissue and whole-body disturbance of static electric field covering the earth, with an ultrasensitive vibroacoustic sensor that passively harvests audible and inaudible biomechanical vibrations generated by the body. The electric potential/vibroacoustic cause-effect combination results in motion amplification so one can literarily see and feel from a distance heart, lung and gut activity cycles and for COVID-19 detect subtle vibrational changes in upper respiratory tract (sinuses, nose, and throat) and lower respiratory tract (windpipe and lungs) through clothing.

Without wishing to be bound by theory, brain rhythms—as recorded in the local field potential (LFP) or scalp electroencephalogram (EEG)—are believed to play a critical role in coordinating brain networks. By modulating neural excitability, these rhythmic fluctuations provide an effective means to control the timing of neuronal firing. Oscillatory rhythms have been categorized into different frequency bands (e.g., theta [4-10 Hz], gamma [30-80 Hz]) and associated with many functions: the theta band with memory, plasticity, and navigation the gamma band with local coupling and competition. In addition, gamma and high-gamma (80-200 Hz) activity have been identified as surrogate markers of neuronal firing, observable in the EEG and LFP.

In general, lower frequency rhythms engage larger brain areas and modulate spatially localized fast activity. For example, the phase of low frequency rhythms has been shown to modulate and coordinate neural spiking via local circuit mechanisms that provide discrete windows of increased excitability.

Vagal nerve stimulation (VNS) is used as treatment in depression and epilepsy. A positron emission tomography (PET) study has shown decreased blood flow to limbic brain regions during direct (cervical) VNS. Another functional magnetic resonance imaging (fMRI) study has shown significant deactivation of limbic brain regions during transcutaneous VNS. In this procedure electrical stimulus is applied over the inner part of the left tragus and hence the auricular branch of the vagus.

### Vagal Nerve Stimulation (VNS)

The vagus nerve serves as the body's superhighway, carrying information between the brain and the internal organs and controlling the body's response in times of rest and relaxation. The large nerve originates in the brain and branches out in multiple directions to the neck and torso, where it's responsible for actions such as carrying sensory information from the skin of the ear, controlling the muscles that you use to swallow and speak and influencing your immune system. Since this nerve is the primary communicator between the brain, heart, and digestive organs irregularities can lead to painful physical and mental health consequences. For this reason, it's the site of potential treatments for various disorders and conditions connected to the brain and body. VNS dampens sympathetic nerve activity that supplies many organs and where there is a dual sympathetic and vagal nerve. In this case, the vagus nerve exerts an opposing effect to the effects of the sympathetic nerves. There is also a sensory component of the vagus nerve that conveys information about the functioning and well-being of the visceral organs to the brain. The regions of the brain that receive this input are involved in regulating not only visceral organ functions, like the heart pumping blood to the body and how much oxygen is circulating through the blood vessels, but also modifying central autonomic and limbic systems. One benefit of VNS stimulation may be its activation of the afferent nerve fibers those going to the brain. The afferent fibers can exert widespread effects on the autonomic, reticular, and limbic areas of the brain to affect mood, alertness and attention, and emotional responses to our experience.

Irregularities in the vagus nerve can cause tremendous distress in physical and emotional health. Physical consequences can include irritable bowel syndrome (IBS), heart burn or GERD, nausea or vomiting, fainting, tinnitus, tachycardia, auto-immune disorders, seizures, and migraines. Mental health consequences include fatigue, depression, panic attacks, or a classic alternation between feeling overwhelmed and shut-down.

Vagus nerve stimulation (VNS) has been around since the 1990s. Doctors isolate the nerve typically in the neck, where it is most accessible and surgically attach electrodes directly to the nerve to help promote a resting and body restorative state. Doctors must set it at a specific frequency, determine how often the electric signals will fire, and regulate the activity, even after the device is installed in the body. Research on Vagus Nerve Stimulation suggests promising results for: anxiety, PTSD, heart disease, auto-immune disorders and systemic inflammation, memory problems and Alzheimer's disease, depression, migraines, fibromyalgia, tinnitus, thyroid disorders, digestive difficulties such as IBS, colitis, GERD, leaky gut, gastroparesis or coli, Traumatic Brain Injury (TBI)

Natural vagus nerve stimulation can be achieved through a wide range of behaviors that include: Mindfulness practices, loving kindness meditation, and yoga, Slow rhythmic breathing, Applying cold washcloth to the face (diving reflex), Vasalva maneuver (exhaling against a closed airway), Massage, craniosacral therapy, and acupuncture, Positive social connections, Humming, singing, or chanting, Healthy diet with probiotics, Polyvagal Perspectives.

Vagal nerve stimulation (VNS) is a medical treatment that involves delivering electrical impulses to the vagus nerve. It is used as an add-on treatment for certain types of intractable epilepsy and treatment-resistant depression. Stimulation of the vagus nerve has been effective in treating cases of epilepsy that do not respond to medication. Surgeons place an electrode around the right branch of the vagus nerve in the neck, with a battery implanted below the collarbone. The electrode provides regular stimulation to the nerve, which decreases, or in rare cases prevents, the excessive brain activity that causes seizures. Research has also shown that vagus nerve stimulation could be effective for treating psychiatric conditions that don't respond to medication. The FDA has approved vagus nerve stimulation for treatment-resistant depression and for cluster headaches. More recently, researchers have been investigating the vagus nerve's role in treating chronic inflammatory disorders such as sepsis, lung injury, rheumatoid arthritis (RA) and diabetes, according to a 2018 review in the Journal of Inflammation Research (Johnson R L, Wilson C G. A review of vagus nerve stimulation as a therapeutic intervention. J Inflamm Res. 2018; 11:203-213 https://doi.org/10.2147/JIR.S163248). Because the vagus nerve influences the immune system, damage to the nerve may have a role in autoimmune and other disorders. We propose an alternative evidence-based approach for targeted vagal nerve stimulation by adapting the Observe, Orient, Decide, and Act (OODA) Loop, a rapid cycle management strategy. We have developed personalized and targeted vagal nerve stimulation prophylaxis protocol that can be tuned and adapted until a stable desired effect of vagal nerve stimulation is achieved. A combination of vibroacoustic and electric potential resonance frequencies and aperiodic patterns is used in patients with minimum matching risk factors. Patients exceeding threshold risk factors receive updated vagal stimulation intervention. The OODA paradigm provides an effective technique for interfacing personalized health care with clinical practice.

### Gastric and Bladder OODA Loop Stimulation

Similar to the heart, the stomach has electrical activity that orchestrates muscle contractions. Gastroparesis is a condition in which the stomach takes too long to empty its contents. Food and liquid stay in the stomach for a long time, which can lead to symptoms such as nausea, vomiting and abdominal pain. Gastroparesis may potentially contribute to poor glycemic control in diabetics, and in extreme cases, carries a risk of dehydration or malnutrition. Modifying stomach contractions through gastric electrical stimulation (GES)—the equivalent of a gut pacemaker—holds potential for treating not only gastric motor disorders, but also eating disorders.

Gastric electrical stimulation may be considered instead of more invasive procedures, such as stomach banding, that are used to treat obesity along with dieting and other measures. Gastric stimulation involves using a pacemaker-like device to stimulate the vagus nerve and affect stomach muscles involved in digestion. The stimulation may make people feel full longer, or change how quickly food passes through the stomach. Gastric stimulation can be used to help control gastroparesis—delayed stomach-emptying of solid food—which causes bloating, distension, nausea and/or vomiting.

A gastric stimulator is a small device that is like a pacemaker for the stomach. It is implanted in the abdomen and delivers mild electrical impulses that stimulate the stomach. This allows food to move through the stomach more normally, relieving the symptoms of gastroparesis.

In one embodiment, a vibroacoustic and electric potential subsystem is non-invasively attached to the vagal nerve and the stomach to first collect gut motility resonance frequency data. A personalized and targeted vagal nerve stimulation prophylaxis protocol that can be tuned and adapted until a stable desired effect of vagal nerve stimulation is achieved is activated. A combination of vibroacoustic and electric potential resonance frequencies is used in patients with minimum matching risk factors. Patients exceeding threshold risk factors receive updated vagal stimulation intervention. The OODA paradigm provides an effective technique for interfacing personalized health care with clinical practice.

### Vibro-Electrical Stimulation for Overactive Bladder

Electrical stimulation may give better control over the muscles in the bladder, a sac-shaped organ that holds urine. Traditionally, a mild electric current is used to treat overactive bladder (OAB) and ease strong urge to pee. In sacral nerve stimulation (SNS), a pacemaker-like device is placed in the back at the base of the spine, the site of the sacral nerve, which carries signals between the bladder, spinal cord, and brain that tell you when you need to urinate. SNS interrupts those signals. SNS can cause side effects, including: pain, wire movement, infection, temporary electric shock-like feeling, bleeding at implant site. The device may also stop working. Up to ⅔ of people who have SNS will need another surgery within 5 years to fix the implant or to replace the battery.

Alternatively, non-surgical percutaneous tibial nerve stimulation (PTNS) is attempted, whereby a thin needle in inserted under the skin of the ankle near the tibial nerve. A stimulator on the outside of your body sends electrical impulses through the needle to the nerve, and on to other nerves in your spine that control the bladder.

Transcutaneous electrical nerve stimulation (TENS). This procedure strengthens the muscles that control urination. Thin wires are placed inside the vagina in females, or in the buttocks, if male. The system delivers pulses of electricity that stimulate the bladder muscles to make them stronger.

In one embodiment, a vibroacoustic and electric potential subsystem is non-invasively attached to the vagal nerve, tibial nerve, vagina and or buttocks and the bladder to first collect resonance frequency data. A personalized and targeted vagal nerve stimulation prophylaxis protocol that can be tuned and adapted until a stable desired effect of vagal and tibial nerves stimulation is achieved is activated. A combination of vibroacoustic and electric potential resonance frequencies is used in patients with minimum matching risk factors. Patients exceeding threshold risk factors receive updated vagal stimulation intervention. The OODA paradigm provides an effective technique for interfacing personalized health care with clinical practice.

### Placenta and Uterus OODA Loop Stimulation

The placenta is arguably the most important organ of the body, but paradoxically the most poorly understood. During its transient existence during growth and development of the fetus, it performs actions that are later taken on by diverse separate organs, including the lungs, liver, gut, kidneys and endocrine glands. Its principal function is to supply the fetus, and in particular, the fetal brain, with oxygen and nutrients. The placenta is structurally adapted to achieve this, possessing a large surface area for exchange and a thin interhaemal membrane separating the maternal and fetal circulations. In addition, it adopts other strategies that are key to facilitating transfer, including remodeling of the maternal uterine arteries that supply the placenta to ensure optimal perfusion. Furthermore, placental hormones have profound effects on maternal metabolism, initially building up her energy reserves and then releasing these to support fetal growth in later pregnancy and lactation postnatally. Bipedalism has posed unique hemodynamic challenges to the placental circulation, as pressure applied to the vena cava by the pregnant uterus may compromise venous return to the heart. These challenges, along with the immune interactions involved in maternal arterial remodeling, may explain complications of pregnancy that are almost unique to the human, including pre-eclampsia. Such complications may represent a trade-off against the provision for a large fetal brain.

Labor induction also known as inducing labor is the stimulation of uterine contractions during pregnancy before labor begins on its own to achieve a vaginal birth. Labor is a process through which the fetus moves from the intrauterine to the extrauterine environment. It is a clinical diagnosis defined as the initiation and perpetuation of uterine contractions with the goal of producing progressive cervical effacement and dilation. Induction of labor refers to the process whereby uterine contractions are initiated by medical or surgical means before the onset of spontaneous labor.

Over the past few years, there has been an increasing awareness that if the cervix is unfavorable, a successful vaginal birth is less likely. Various scoring systems for cervical assessment have been introduced. In 1964, Bishop systematically evaluated a group of multiparous women for elective induction and developed a standardized cervical scoring system. The Bishop score helps delineate patients who would be most likely to achieve a successful induction. The duration of labor is inversely correlated with the Bishop score; a score that exceeds 8 describes the patient most likely to achieve a successful vaginal birth. Bishop scores of less than 6 usually require that a cervical ripening method be used before other methods.

A health care provider might recommend labor induction for various reasons, primarily when there's concern for a mother's health or a baby's health. Induction of labor is common in obstetric practice. According to the most current studies, the rate varies from 9.5 to 33.7 percent of all pregnancies annually. In the absence of a ripe or favorable cervix, a successful vaginal birth is less likely. Therefore, cervical ripening or preparedness for induction should be assessed before a regimen is selected. Assessment is accomplished by calculating a Bishop score. When the Bishop score is less than 6, it is recommended that a cervical ripening agent be used before labor induction. Nonpharmacologic approaches to cervical ripening and labor induction have included herbal compounds, castor oil, hot baths, enemas, sexual intercourse, breast stimulation, acupuncture, acupressure, transcutaneous nerve stimulation, and mechanical and surgical modalities. Of these nonpharmacologic methods, only the mechanical and surgical methods have proven efficacy for cervical ripening or induction of labor.

All mechanical modalities share a similar mechanism of action-namely, some form of local pressure that stimulates the release of prostaglandins. Risks:

Mechanical induction of labor may cause vaginal or placental bleeding and be life-threatening to the mother or newborn. It may cause mother or the baby to get an infection. Amniotic fluid may leak into the mother's blood and cause her to have lung, heart, and bleeding problems. Mechanical induction may increase risk for a cesarean section (C-section). The amniotic fluid sac may break before the cervix softens and thins. The newborn baby's heartbeat may slow, putting the baby at risk for problems. There is a risk that the mother's uterus could rupture if the mother has had a C-section before.

In one embodiment, a vibroacoustic and electric potential subsystem is non-invasively attached to the vagal nerve and the cervix/uterus to first collect resonance frequency data. A personalized and targeted vagal nerve stimulation prophylaxis protocol that can be tuned and adapted until a stable desired effect of vagal and tibial nerves stimulation is achieved is activated. A combination of vibroacoustic and electric potential resonance frequencies is used in patients with minimum matching risk factors. Patients exceeding threshold risk factors receive updated vagal stimulation intervention. The OODA paradigm provides an effective technique for interfacing personalized health care with clinical practice.

### Autonomic Nervous System Stimulation

The use of ‘OM’ chanting for meditation is well known. Effective ‘OM’ chanting is associated with the experience of vibration sensation around the ears. It is expected that such a sensation is also transmitted through the auricular branch of the vagus nerve. We therefore hypothesized that like transcutaneous VNS, ‘OM’ chanting too produces limbic deactivation. Specifically, we predicted that ‘OM’ chanting would evoke similar neurohemodynamic correlates, deactivation of the limbic brain regions, amygdala, hippocampus, parahippocampal gyrus, insula, orbitofrontal and anterior cingulate cortices and thalamus) as were found in the previous study.

The devices, methods and systems of the present technology, in certain embodiments, can harvest autonomic nervous system vibroacoustic multi-modal biosignals separately or together with central nervous data. Autonomous data collection is well understood and follows percussive auscultation as a precedence. Central nervous system auscultation is unique. The skull bones are layered with a thinner, denser inner part that is separated from a thicker, tougher outer bone by a soft layer of cancellous tissue (diploe), each with varying coefficients of absorption and transference for acoustic vibrations. There are some indications that the anatomical and physiological differences between human skulls might produce unique resonances (i.e., additive energy at a specific frequency) and spectral filtering for incoming sounds. The devices, systems, and methods of the present technology harvests audible and inaudible vibroacoustic signals and quantifies the impact of unique filtering by an individual's skull regarding some individual characteristics of skulls). Autonomous and central nervous system vibroacoustic data collection may enable the deconvolution, and qualitative and quantitative characterization of physical, neuropsycho functional health state, behavioral, intelligence and prediction of the impact of individual variability relating to environmental and social exposures, on neurocognitive outcomes, trait EI, ability EI, and emotion information processing—may contribute to effective emotion-related performance and provide initial evidence supporting its usefulness in predicting EI-related outcomes—namely an alternative data-driven Theory of Mind concept.

Everything in the universe has energy and vibrates at different frequencies. Vibrations are defined as repeated oscillatory movements of a body. The transmission of inaudible and audible vibration energy can be localized or generalized. They can be transmitted through the air without contact and via structural surfaces, water and the ground. From the point of view of physics, vibrations can be differentiated on the basis of frequency, wavelength, amplitude of the oscillation, velocity and acceleration. As far as submarine structural health, two risk factors are dominant: the first involves low frequency vibrations (high energy inaudible sound, or infrasound <20 Hz), while the second involves high frequency vibrations (audible and inaudible percussion, 20-160 kHz).

Deep inside the inner ear, within a little nautilus-shaped bone called the cochlea, tiny little hairs vibrate to transform sound into brain signals. Sound waves flowing around in the cochlea don't just hit the hairs and go away, but rather they bounce around within the head-interacting with skull bones. Every object in the world vibrates at what is known as its “natural frequency,” the inside and outside of the skull included, and these vibrations affect the sound waves that the hairs in the cochlea have resonance with.

The natural frequency of the head is a combination of the skull's size, density, hair and shape, meaning that the vibrations of your skull are ever-so-slightly different than the person next to you. The natural vibrational frequency in people's heads is in the range from about 30 to 70 Hz (30-70 vibrations per second), with women's heads tending to vibrate faster than men's.

The variation of people's vibrating skulls is potentially predictive of intelligence, emotional iq, music preference, rate of loss of hearing, risk of dementia disease and determines the impact and rate of aging.

The skull is a resonant chamber that is tuned and modified by the cochlea. Simple and complex, integer/fractal-based ratios between the frequency of the skull, and the prominent frequencies in language, speech and voice patterns, used in a pieces of music, will tend to make that music sound somewhat louder and richer to a listener. In this way it is possible, to determine with quantitative accuracy how resonance frequency ratios to the fundamental frequencies of the skull influence experienced acoustic distortions and make music/language impenetrable or unattractive to an individual.

The ancient Tibetan metaphysical texts state that all sound is music, all music is mantra, and mantra is the essence of all sound. Mantra is a pattern of sound or sound vibration that is based upon primordial sound structures. By their sheer inherent potency and disciplined execution, these concentrated essential energies bring about direct spiritual phenomenon.

Through the use of ritual and mantric power, the Tibetans use sound to effect a specific change in the individual and the environment. Nuances of hisses, static, pure beats all wash over the auditory field, activating a sort of sensory overload that heightens other forms of mentation. Increased ideation, mental visions, and the emergence of memories bring listening beyond melody into the space. Images appear unbidden, and a trans-cranial cinema emerges from the fully engaged senses.

The sound artist Kim Cascone has developed a program that is characterized as theory-based, theoretical, abstractive, suppositious aural meditation program. He has designed what he calls a ‘Subtle Listening Seminar’ which engages people in developing a better understanding of the nuances of sound. Subtle Listening is a mode of listening where one's imagination is open to the sound world around them, helping their inner ear and outer world intersect. The Subtle Listening workshop is an ongoing workshop for musicians, media artists, filmmakers, composers, producers, sound designers, or any type of artist who wants to sharpen their listening skills. The workshop uses a wide range of techniques culled from Jungian psychology, Hermetic philosophy, paradox and Buddhist meditation, etc. Through guided meditation and various types of listening exercises, participants learn techniques they can use any time to help heighten their sensitivity to the sounds around them, and to bring out the depth of experience that is possible when humans interact with the natural soundscape around them. The human skull is a vibroacoustic chamber, a place for enhanced stimulation for an aural engagement that can lead to spaces in which it is possible to work directly with the mental states and symbolic imagery evoked through a dutiful attention to the art of merging listening, feeling and being.

Embodiments of the devices, methods and systems of the present technology provide for the quantification of the entire vibroacoustic soundfield, combined with data-driven insight on the interaction of sound source and observer resonances we can include, tune and target binaural sounds different audio tracks to affect an active change in the brainwave patterns of the listener, allowing these intentional therapeutic/re-wiring/brain-activity enhancing compositions to be what Stephan Schwartz, one of the scientists active in studying Remote Viewing, calls a “ground for working” with the ambient mental field.

The devices, methods and systems of the present technology provide for vibroacoustic soundfield bio-feedback creations that are customizable, psyche-summoning sound sculptures that invite “a mode of listening where one's imagination is open to the sound world around them, helping their inner ear and outer world intersect.” These vibroacoustic soundfields act as substructures that bring about visualized equations of symbolic exchange, with sound acting as the ambient bed on which a lucid mental field emerges in which to work.

Resonances occur naturally, when there are two or more energy storage modes with coupling between them. In mechanical structures the common modes are potential and kinetic energy. In electrical it's E-field and H-field energies. Resonances have been used for measurements in many fields. Embodiments of the devices, methods and systems of the present technology provide for means of probing devices passively or actively (vibroacoustically and electromagnetically), looking for resonant signatures (or munition fingerprints) which are compared against known knowns, or digital twin simulations.

### Binaural Fusion

Binaural fusion or binaural integration is a cognitive process that involves the combination of different auditory information presented binaurally, or to each ear. In humans, this process is essential in understanding speech as one ear may pick up more information about the speech stimuli than the other. The frequency resonances of the skull therefore have an essential role in the understanding and appreciation of the vibroacoustic soundfield around us.

The process of binaural fusion is important for computing the location of sound sources in the horizontal plane (sound localization), and it is important for sound segregation. Sound segregation refers to the ability to identify acoustic components from one or more sound sources. The binaural auditory system is highly dynamic and capable of rapidly adjusting tuning properties depending on the context in which sounds are heard. Each eardrum moves one-dimensionally; the auditory brain analyzes and compares movements of both eardrums to extract physical cues and synthesize auditory objects.

When stimulation from a sound reaches the ear, the eardrum deflects in a mechanical fashion, and the three middle ear bones (ossicles) transmit the mechanical signal to the cochlea, where hair cells transform the mechanical signal into an electrical signal. The auditory nerve, also called the cochlear nerve, then transmits action potentials to the central auditory nervous system 3.

In binaural fusion, inputs from both ears integrate and fuse to create a complete auditory picture at the brainstem. Therefore, the signals sent to the central auditory nervous system are representative of this complete picture, integrated information from both ears instead of a single ear. The binaural squelch effect is a result of nuclei of the brainstem processing timing, amplitude, and spectral differences between the two ears. Sounds are integrated and then separated into auditory objects. For this effect to take place, neural integration from both sides is required.

### Binaural Sound

Binaural beats are considered auditory illusions. When you hear two tones, one in each ear, that are slightly different in frequency, your brain processes a beat at the difference of the frequencies. This is called a binaural beat. Here's an example: Let's say you're listening to a sound in your left ear that's at a frequency of 84 Hertz (Hz), and in your right ear, you're listening to a sound that's at a frequency of 105 Hz. Your brain gradually falls into synchrony with the difference—or 21 Hz. Instead of hearing two different tones, you instead hear 3 tones—a tone at 21 Hz (in addition to the two tones given to each ear, 84 Hz and 105 Hz).

For a binaural beat to work, the two tones must have frequencies less than about 1000 Hz, and the difference between the two tones can't be more than about 30 Hz. The tones also have to be listened to separately, one through each ear. Binaural beats have been explored in music and are sometimes used to help tune instruments, such as pianos and organs. More recently, they have been connected to potential health benefits.

The ear functions to analyze and encode a sound's dimensions. Binaural fusion is responsible for avoiding the creation of multiple sound images from a sound source and its reflections.

The central auditory system converges inputs from both ears (inputs contain no explicit spatial information) onto single neurons within the brainstem. This system contains many subcortical sites that have integrative functions. The auditory nuclei collect, integrate, and analyze afferent supply, the outcome is a representation of auditory space (3). The subcortical auditory nuclei are responsible for extraction and analysis of dimensions of sounds (5).

The integration of a sound stimulus involves inline analysis of the frequency (pitch), intensity, and spatial localization of the sound source. Once a sound source has been identified, the cells of lower auditory pathways are specialized to analyze physical sound parameters (3). Summation is observed when the loudness of a sound from one stimulus is perceived as having been doubled when heard by both ears instead of only one. This process of summation is called binaural summation and is the result of different acoustics at each ear, depending on where sound is coming from (4).

The medial superior olive (MSO) contains cells that function in comparing inputs from the left and right cochlear nuclei. The tuning of neurons in the MSO favors low frequencies, whereas those in the lateral superior olive (LSO) favor high frequencies.

### Sound Localization

Sound localization is the ability to correctly identify the directional location of sounds. A sound stimulus localized in the horizontal plane is called azimuth; in the vertical plane it is referred to as elevation. The time, intensity, and spectral differences in the sound arriving at the two ears are used in localization. Localization of low frequency sounds is accomplished by analyzing interaural time difference (ITD). Localization of high frequency sounds is accomplished by analyzing interaural level difference (ILD) (4).

### Mechanism—Binaural Hearing

Action potentials originate in the hair cells of the cochlea and propagate to the brainstem; both the timing of these action potentials and the signal they transmit provide information to the superior olivary complex (SOC) about the orientation of sound in space. The processing and propagation of action potentials is rapid, and therefore, information about the timing of the sounds that were heard, which is crucial to binaural processing, is conserved. Each eardrum moves in one dimension, and the auditory brain analyzes and compares the movements of both eardrums in order to synthesize auditory objects (3). This integration of information from both ears is the essence of binaural fusion. The binaural system of hearing involves sound localization in the horizontal plane, contrasting with the monaural system of hearing, which involves sound localization in the vertical plane (3).

### Superior Olivary Complex

The primary stage of binaural fusion, the processing of binaural signals, occurs at the SOC, where afferent fibers of the left and right auditory pathways first converge. This processing occurs because of the interaction of excitatory and inhibitory inputs in the LSO and MSO (1,3). The SOC processes and integrates binaural information, in the form of ITD and ILD, entering the brainstem from the cochleae. This initial processing of ILD and ITD is regulated by GABAB receptors (1).

### ITD and ILD

The auditory space of binaural hearing is constructed based on the analysis of differences in two different binaural cues in the horizontal plane: sound level, or ILD, and arrival time at the two ears, or ITD, which allow for the comparison of the sound heard at each eardrum (1,3). ITD is processed in the MSO and results from sounds arriving earlier at one ear than the other; this occurs when the sound does not arise from directly in front or directly behind the hearer. ILD is processed in the LSO and results from the shadowing effect that is produced at the ear that is farther from the sound source. Outputs from the SOC are targeted to the dorsal nucleus of the lateral lemniscus as well as the IC(3).

### Lateral Superior Olive

LSO neurons are excited by inputs from one ear and inhibited by inputs from the other, and are therefore referred to as IE neurons. Excitatory inputs are received at the LSO from spherical bushy cells of the ipsilateral cochlear nucleus, which combine inputs coming from several auditory nerve fibers. Inhibitory inputs are received at the LSO from globular bushy cells of the contralateral cochlear nucleus (3).

### Medial Superior Olive

MSO neurons are excited bilaterally, meaning that they are excited by inputs from both ears, and they are therefore referred to as EE neurons (3). Fibers from the left cochlear nucleus terminate on the left of MSO neurons, and fibers from the right cochlear nucleus terminate on the right of MSO neurons (5). Excitatory inputs to the MSO from spherical bushy cells are mediated by glutamate, and inhibitory inputs to the MSO from globular bushy cells are mediated by glycine. MSO neurons extract ITD information from binaural inputs and resolve small differences in the time of arrival of sounds at each ear (3). Outputs from the MSO and LSO are sent via the lateral lemniscus to the IC, which integrates the spatial localization of sound. In the IC, acoustic cues have been processed and filtered into separate streams, forming the basis of auditory object recognition (3).

### Binaural Beats Health Benefits

Becoming a master at meditation is not easy. Meditation is the practice of calming the mind and tuning down the number of random thoughts that pass through it. A regular meditation practice has been shown to reduce stress and anxiety, slow down the rate of brain aging and memory loss, promote emotional health, and lengthen attention span. Practicing meditation regularly can be quite difficult, so people have looked to technology for help.

While most studies on the effects of binaural beats have been small, there are several that provide evidence that this auditory illusion does indeed have health benefits, especially related to anxiety, mood, and performance. Even without an established empirical basis or approach, binaural beats are claimed to induce the same mental state associated with deep meditation practice, but much more quickly. In effect, binaural beats are said to: reduce anxiety, increase focus and concentration, lower stress, increase relaxation, foster positive moods, promote creativity, help manage pain.

Binaural beats between about 1 and 30 Hz (Note the resonance limit) are alleged to create the same brainwave pattern that one would experience during meditation. When you listen to a sound with a certain frequency, your brain waves will synchronize with that frequency. The theory is that binaural beats can help create the frequency needed for your brain to create the same waves commonly experienced during a meditation practice. The use of binaural beats in this way is sometimes called brainwave entrainment technology.

Currently there is no empiric way to determine and personalize binaural beat self-treatment. All you need to experiment with binaural beats is a binaural beat audio and a pair of headphones or earbuds. Audio files of binaural beats are available online, such as on YouTube, or you can purchase CDs or download audio files directly to your mp3 player or other device. As mentioned earlier, for a binaural beat to work, the two tones must have frequencies of less than about 1000 Hz, and the difference between the two tones can't be more than about 30 Hz.

Current practice is that you will need to decide and determine which brainwave fits your desired state. In general: Binaural beats in the delta (about 1 to 4 Hz) range have been associated with deep sleep and relaxation. Binaural beats in the theta (about 4 to 8 Hz) range are linked to REM sleep, reduced anxiety, relaxation, as well as meditative and creative states. Binaural beats in the alpha frequencies (about 8 to 13 Hz) are thought to encourage relaxation, promote positivity, and decrease anxiety. Binaural beats in the lower beta frequencies (about 14 to 30 Hz) have been linked to increased concentration and alertness, problem solving, and improved memory.

Volume, duration of exposure and timing between binaural beat exposure sessions is guesswork based on individual preferences rather than health benefit. You have to experiment with the length of time you listen to the binaural beats to find out what works for you. For example, if you're experiencing high levels of anxiety or stress, you may want to listen to the audio for longer. Use of headphones with eyes closed is recommended for beneficial binaural beats effects.

One blinded study in 29 people found that listening to binaural beats in the beta range (about 16 and 24 Hz) was associated with both improved performance on a given task as well as a reduction in negative moods compared to listening to binaural beats in the theta and delta (about 1.5 and 4 Hz) range or to simple white noise.

Another controlled study in roughly 100 people about to undergo surgery also found that binaural beats were able to significantly reduce pre-operative anxiety compared to similar audio without the binaural tones and no audio at all. In the study, anxiety levels were cut in half for people who listened to the binaural beat audio.

Another uncontrolled study asked eight adults to listen to a binaural beat CD with delta (about 1 to 4 Hz) beat frequencies for 60 days straight. The participants filled out surveys before and after the 60-day period that asked questions about their mood and quality of life. The results of the study found that listening to binaural beats for 60 days significantly reduced anxiety and increased the overall quality of life of these participants. Since the study was small, uncontrolled, and relied on patient surveys to collect data, larger studies will be needed to confirm these effects.

One larger and more recent randomized and controlled trial looked at the use of binaural beats in 291 patients admitted to the emergency department at a hospital. The researchers observed significant decreases in anxiety levels in patients exposed to audio with embedded binaural beats compared to those who listened to audio without binaural beats or no audio at all (headphones only).

There are no known side effects to listening to binaural beats. However, lengthy exposure to sounds at or above about 85 decibels can cause hearing loss over time. More research is needed to see if there are any side effects to listening to binaural beats over a long period of time.

In certain embodiments, the present technology provides for a method of personalizing audio, audio-visual and audio-tactile media by: Applying a sweep-frequency stimulation to a subject with a bandwidth of about 0.01 Hz to 80 KHz; Measuring the damping, resonant and reflective responses of the subject to obtain a resonant frequency-response function; and Applying the resonant frequency-response function to an audio program thus selectively enhancing or attenuating the energy content of particular frequency bands of the audio program.

In another embodiment, the present technology provides for the compilation of a personalized library of sounds from about 0.01 Hz to 80 kHz by stimulating the subject with stimuli from a vibroacoustic sound library, measuring brain and skull passive vibroacoustic responses, measuring brain electrical potentials, correlating the vibroacoustic and electrical potential measurements to desired or undesired psychological or physiological responses, and creating a subject specific sound library to selectively attenuate or enhance the undesired, or desired responses, respectively, when played to the subject.

In still another embodiment, the present technology provides for a tinnitus treatment comprising: determining the frequency and phase of the perceived tinnitus sound, applying a phase inverted acoustic signal to cancel the perceived signal.

In still another embodiment, the present technology provides for a method of tuning binaural beat audio stimulation by: stimulating the subject with stimuli from a binaural sound library, measuring brain and skull passive vibroacoustic responses, measuring brain electrical potentials, correlating the vibroacoustic and electrical potential measurements to desired or undesired psychological or physiological responses, and creating a subject specific sound library to selectively attenuate or enhance the undesired, or desired responses, respectively, when played to the subject.

In yet another embodiment, the present technology provides for a method of suppressing a subject's default mode network activity by having a subject listen to sounds from a subject specific sound library that has been selected by the method of tuning binaural beat audio stimulation.

In yet another embodiment, the present technology provides for a method of exposing a subject to binaural stimulation involving the swapping of the lower frequency binaural beat signal from one opposing ear to the other at a predetermined frequency, where the frequency of swapping is from about 0.001 Hz to 0.005 Hz, about 0.005 to 0.01 Hz, about 0.01 Hz to 0.05 Hz, about 0.05 Hz to 0.1 Hz, about 0.1 Hz to 0.5 Hz, about 0.5 Hz to 1 Hz, about 1 Hz to 5 Hz, about 5 Hz to 50 Hz, about 50 Hz to 200 Hz, about 200 Hz to 500 Hz, or about 500 Hz to 1000 Hz.

In certain embodiments, the binaural beat comprises a lower frequency signal and a higher frequency signal which are applied alternatingly to the right and left ear of the subject.

The binaural beats applications: With several human studies to back up the health claims, binaural beats appear to be a promising tool in the fight against anxiety, stress, and negative mental states. Research has found that listening daily to CDs or audio files with binaural beats has positive effects on: anxiety, memory, mood, creativity, attention. Binaural beats won't work for everyone, and they aren't considered a cure for any particular condition. However, they might offer a perfect escape for those interested in relaxing, sleeping more peacefully, or entering a meditative state.

### Additional Applications

Besides meditation and wellbeing use cases—skull acoustic chamber vibroacoustic, vibrometry and electric potential evaluation has added health benefits indicated by hearing loss and or changes in the skull vibroacoustic biofield signature

### Types/Causes of Hearing Loss

Hearing loss is caused by many factors, most frequently from natural aging or exposure to loud noise. The most common causes of hearing loss are: Aging, Noise exposure, Head trauma, Virus or disease, Genetics, Ototoxicity.

There are three types of hearing loss—sensorineural hearing loss, conductive hearing loss, and mixed hearing loss.

Sensorineural hearing loss: Sensorineural hearing loss is the most common type of hearing loss. It occurs when the inner ear nerves and hair cells are damaged—perhaps due to age, noise damage or something else. Sensorineural hearing loss impacts the pathways from your inner ear to your brain. Most times, sensorineural hearing loss cannot be corrected medically or surgically, but can be treated and helped with the use of hearing aids.

Sensorineural hearing loss can be caused by: aging, injury, excessive noise exposure, Viral infections (such as measles or mumps), shingles, ototoxic drugs (medications that damage hearing), meningitis, diabetes, stroke, high fever or elevated body temperature, Ménière's disease (a disorder of the inner ear that can affect hearing and balance), acoustic tumors, heredity, obesity, smoking, hypertension.

Conductive hearing loss: Conductive hearing loss is typically the result of obstructions in the outer or middle ear perhaps due to fluid, tumors, earwax or even ear formation. This obstruction prevents sound from getting to the inner ear. Conductive hearing loss can often be treated surgically or with medicine.

Conductive hearing loss can be caused by: infections of the ear canal or middle ear resulting in fluid or pus buildup, perforation or scarring of the eardrum, wax buildup, dislocation of the middle ear bones (ossicles), foreign object in the ear canal, otosclerosis (an abnormal bone growth in the middle ear) and abnormal growths or tumors.

Mixed hearing loss: Mixed hearing loss is a combination of sensorineural and conductive hearing loss.

Hearing loss and rare diseases: Many rare diseases cause hearing loss. Scientists have identified 7,000 diseases, like Myhre syndrome, that are considered rare. As defined in the U.S by the Orphan Drug Act of 1983, rare diseases each affect fewer than 200,000 people. However, up to 30 million Americans live with a rare disease. Many, but not all, have been traced at least in part to genes, with signs that appear at birth or early in life. At least 400 rare syndromes include hearing loss as a symptom, according to BabyHearing.org. These rare syndromes can lead to different types of hearing loss, the main types being sensorineural and conductive. At least 400 rare syndromes include hearing loss as a symptom. The degree of loss can vary widely from person to person. For some people, hearing aids will be sufficient. For others, cochlear implants and/or learning American Sign Language will be recommended. In many cases, a rare disease can cause multiple anatomical and functional changes in the ears. A prime example of this is Turner syndrome. Hearing loss may be apparent at birth or soon after. Because of state programs aided by the federal government, nearly all American babies have a hearing test within the first month of life. About two or three out of every 1,000 newborns in the U.S. have a detectable hearing loss in one or both ears. Hearing loss may be a sign of a rare disease.

Babies with Mondini dysplasia, for example, are born with one and a half coils in the cochlea instead of the standard two, in either one or both ears. Most children with this condition have profound hearing loss. They may need a surgical repair, as well as a cochlear implant, but some can benefit from hearing aids. Babies with KID syndrome, Donnai-Barrow syndrome, and Wildervanck syndrome—among other rare diseases—may have hearing loss.

Sometimes the loss is not present at birth but develops soon after. Babies with the most common and severe form of Krabbe disease develop symptoms in the first six months, which include fevers, muscle weakness and hearing and vision loss.

Later occurring hearing loss: Hearing loss often comes much later. People with Alport syndrome, for example, often lose hearing in late childhood or early adolescence and may be treated with hearing aids. Similarly, people with Alstrom syndrome tend to have progressive hearing loss in both ears that may begin in childhood and be treated with hearing aids.

Other notable rare disorders linked to hearing loss: Usher syndrome includes three types of hearing loss, depending on the onset and severity of symptoms.

Auditory neuropathy spectrum disorder can appear at any age. Although it runs in some families, it can occur in people with no family history. In this disorder, signals from the inner ear to the brain are not transmitted properly, which leads to mild to severe hearing loss.

Waardenburg syndrome is a group of six genetic conditions that in at least 80 percent of patients involves hearing loss or deafness. People with this syndrome may also have pale blue eyes, different colored eyes, or two colors within one eye; a white forelock (hair just above the forehead); or gray hair early in life.

Vogt-Koyanagi-Haradi disease is an autoimmune disease that causes chronic inflammation of melanocytes, specialized cells that give skin, hair, and eyes their color. Because melanin occurs in the inner ear as well, the early symptoms of Vogt-Koyanagi-Haradi disease may include distorted hearing (dysacusis), ringing in the ears (tinnitus), and a spinning sensation (vertigo). Although most people with this illness eventually develop hearing loss, it may be mild enough to manage with hearing aids.

In Cogan's syndrome, similarly, the immune system attacks the tissues of the eyes and inner ears.

Children with Carpenter syndrome may be of normal intelligence but it is common for them to have an intellectual disability and sometimes hearing loss.

At least 80 percent of people with Myhre syndrome have a hearing impairment, as well as intellectual disability and stiff joints.

Binaural fusion abnormalities in autism. Current research is being performed on the dysfunction of binaural fusion in individuals with autism. The neurological disorder autism is associated with many symptoms of impaired brain function, including the degradation of hearing, both unilateral and bilateral. Individuals with autism who experience hearing loss maintain symptoms such as difficulty listening to background noise and impairments in sound localization. Both the ability to distinguish particular speakers from background noise and the process of sound localization are key products of binaural fusion. They are particularly related to the proper function of the SOC, and there is increasing evidence that morphological abnormalities within the brainstem, namely in the SOC, of autistic individuals are a cause of the hearing difficulties. The neurons of the MSO of individuals with autism display atypical anatomical features, including atypical cell shape and orientation of the cell body as well as stellate and fusiform formations. Data also suggests that neurons of the LSO and MNTB contain distinct dysmorphology in autistic individuals, such as irregular stellate and fusiform shapes and a smaller than normal size. Moreover, a significant depletion of SOC neurons is seen in the brainstem of autistic individuals. All of these structures play a crucial role in the proper functioning of binaural fusion, so their dysmorphology may be at least partially responsible for the incidence of these auditory symptoms in autistic patients (9).

Ménière's disease is a disorder of the inner ear that causes severe dizziness (vertigo), ringing in the ears (tinnitus), hearing loss, and a feeling of fullness or congestion in the ear. Ménière's disease usually affects only one ear. Attacks of dizziness may come on suddenly or after a short period of tinnitus or muffled hearing. Some people will have single attacks of dizziness separated by long periods of time. Others may experience many attacks closer together over a number of days.

Some people with Ménière's disease have vertigo so extreme that they lose their balance and fall. These episodes are called “drop attacks. Ménière's disease can develop at any age, but it is more likely to happen to adults between 40 and 60 years of age. The National Institute on Deafness and Other Communication Disorders (NIDCD) estimates that approximately 615,000 individuals in the United States are currently diagnosed with Ménière's disease and that 45,500 cases are newly diagnosed each year.

The symptoms of Ménière's disease are caused by the buildup of fluid in the compartments of the inner ear, called the labyrinth. The labyrinth contains the organs of balance (the semicircular canals and otolithic organs) and of hearing (the cochlea). It has two sections: the bony labyrinth and the membranous labyrinth. The membranous labyrinth is filled with a fluid called endolymph that, in the balance organs, stimulates receptors as the body moves. The receptors then send signals to the brain about the body's position and movement. In the cochlea, fluid is compressed in response to sound vibrations, which stimulates sensory cells that send signals to the brain.

In Ménière's disease, the endolymph buildup in the labyrinth interferes with the normal balance and hearing signals between the inner ear and the brain. This abnormality causes vertigo and other symptoms of Ménière's disease. Ménière's disease is most often diagnosed and treated by an otolaryngologist (commonly called an ear, nose, and throat doctor, or ENT). However, there is no definitive test or single symptom that a doctor can use to make the diagnosis. Diagnosis is based upon medical history and the presence of: two or more episodes of vertigo lasting at least 20 minutes each, tinnitus, temporary hearing loss, feeling of fullness in the ear.

Some doctors will perform a hearing test to establish the extent of hearing loss caused by Ménière's disease. To rule out other diseases, a doctor also might request magnetic resonance imaging (MRI) or computed tomography (CT) scans of the brain

### Traumatic Brain Injury Detection

Mild traumatic brain injuries (mTBI) are caused by trauma to the head or neck that results in physiological dysfunction manifest as loss of consciousness, altered mental status, or transient memory loss. It is estimated that 42 million people worldwide suffer some form of mTBI every year and that the majority of them do not seek medical attention. Concussion, a subcategory of mTBI, is thought to be reversible and is often caused by sports. It is estimated that 1.6 to 3.8 million brain injuries occur in sports every year in the USA, the majority of them being mTBI. Elite athletes and warfighters often do not realize that they have been injured because they are so consumed with the task at hand.

### Intracranial Pressure

Intracranial pressure (ICP) is the pressure of the cerebrospinal fluid in the subarachnoid space. Normal values are 7-15 mmHg in a healthy supine adult and −10 mmHg in the standing position. Increased ICP is well documented in moderate and severe forms of traumatic brain injury (TBI) due to gross swelling or mass effect from bleeding. Since the brain exists within a stiff skull, increased ICP can impair cerebral blood flow (CBF) and cause secondary ischemic insult. The symptoms of increased ICP include but are not limited to headache, behavioural problems, nausea, and vision problems, which overlap with the symptoms of mTBI and concussion.

Increased ICP during severe or moderate TBI is a well-known phenomenon due to the mass effect of bleeding or gross swelling of the brain. Changes in ICP can be due to alterations in CBF and autonomic nervous system (ANS) seen in mTBI patients. The primary ANS control center located in the brainstem may be damaged particularly if there is a rotational force applied to the upper cervical spine as seen in head injuries. Direct and indirect measurement of ICP is important to collect noninvasively because the symptoms of intracranial hypertension include but are not limited to headache, behavioral problems, nausea, and vision problems, which overlap with the symptoms of mTBI and concussion. Human and animal data support that there may be increased ICP after mTBI and this increase can remain elevated for several days after injury which is similar to the symptom recovery time reported in humans after sports-related concussion.

### REFERENCES

1. Grothe, Benedikt; Koch, Ursula (2011). “Dynamics of binaural processing in the mammalian sound localization pathway—the role of GABA(B) receptors”. Hearing Research. 279 (1-2): 43-50.
2. Schwartz, Andrew; McDermott, Josh (2012). “Spatial cues alone produce inaccurate sound segregation: The effect of inter aural time differences”. Journal of the Acoustical Society of America. 132 (1): 357-368.
3. Grothe, Benedikt; Pecka, Michael; McAlpine, David (2010). “Mechanisms of sound localization in mammals”. Physiol Rev. 90 (3): 983-1012
4. Tyler, R. S.; Dunn, C. C.; Witt, S. A.; Preece, J. P. (2003). “Update on bilateral cochlear implantation”. Current Opinion in Otolaryngology & Head and Neck Surgery. 11 (5): 388-393.
5. Masterton, R. B. (1992). “Role of the central auditory system in hearing: the new direction”. Trends in Neurosciences. 15 (8): 280-285.
6. Eldredge, D. H.; Miller, J. D. (1971). “Physiology of hearing”. Annu. Rev. Physiol. 33: 281-310.
7. Guinan, J J; Norris, B E; Guinan, S S (1972). “Single auditory units in the superior olivary complex II: Locations of unit categories and tonotopic organization”. Int J Neurosci. 4 (4): 147-166.
8. Forsythe, Ian D. “Excitatory and inhibitory transmission in the superior olivary complex”. 6^{th} Bienniel Symposium Center for neural Science at New York University, Jun. 10-11, 2001 Processing the Auditory Environment. https://www.cns.nvu.edu/events/symposia/abstracts-2001/Forsythe.pdf accessed 16 Mar. 2021.
9. Kulesza Jr., Randy J.; Lukose, Richard; Stevens, Lisa Veith (2011). “Malformation of the human superior olive in autism spectrum disorders”. Brain Research. 1367: 360-371.

### Mind Reading

We recognize words as pictures. As your eyes scan these words, your brain seems to derive their meaning instantaneously. How are we able to recognize and interpret marks on a page so rapidly? Studies confirm that a specialized brain area recognizes printed words as pictures rather than by their meaning.

Focused functional MRI studies of a tiny area of the brain known to be involved in recognizing words, the visual word form area (VWFA), found on the surface of the brain, behind the left ear. The VWFA's right hemisphere analogue is the fusiform face area, which allows us to recognize faces. In young children and people who are illiterate, the VWFA region and the fusiform face area both respond to faces. As people learn to read, the VWFA region is co-opted for word recognition.

In research with participant presented the subjects with a series of real words and made-up words, the nonsense words elicited responses from a wide pool of neurons in the VWFA, whereas distinct subsets of neurons responded to real words. After subjects were trained to recognize pseudo words, however, neurons responded as they did to real words. Because initially the nonsense words had no meaning, neurons must respond to words' orthography-how they look-rather than their meaning.

The predominant model of VWFA function, referred to here as the language model, states that the VWFA has a specific computational role in decoding written forms of words and is considered a crucial node of the brain's language network (10) (11). Consistent with the language model of VWFA function, a large body of evidence has accumulated showing regional activation for orthographic symbols in VWFA, including letters (12) and words (13) (14) compared to a range of visual control stimuli. Additional support for the language node model has been provided by studies examining structural and intrinsic functional connectivity of VWFA. For example, recent studies have shown strong profiles of white-matter 10,11 and functional connectivity 12,13 between VWFA and lateral prefrontal, superior temporal, and inferior parietal regions implicated in language-related functions. These results support the language model by suggesting that the VWFA has privileged connectivity to other nodes of the distributed language network.

Reported data suggest that brain regions involved in speech production display largely parallel activity. For example, early neurophysiological studies of intracranial recordings in monkey have revealed the near-instantaneous and parallel activity of many areas of the brain during manual motor decision tasks. Our “mind-reading” novel approach for non-invasively capturing the real-time vibroacoustic and electric potential biofield activity of the human brain builds on multivariate pattern analysis method, spatiotemporal searchlight representational similarity analysis (ssRSA), to interpret and decode subtle pressure and resonance frequency changes, preference and selectivity directly from the dynamic neural activity of the brain as reconstructed in combined real-time vibroacoustic and electric potential biofield word-association source space. This method is an extension of fMRI-based time-resolved imaging methods. Mind-reading word libraries and feedforward/feedback algorithms are computed based on learned similarities and dissimilarities between modelled representations of unspoken words, rather than just modelling the response stimulated by the words themselves. Initial words for model construction were constrained to a set of Dr. Seuss and Dolch's 400 sight words. Borrowing from educator's perspective, Dr. Seuss' books help children learn to read through repetitive use of sight words. Sight words represent over 50% of all English print media. These high frequency words have an even higher concentration (75% to 90%) in Dr. Seuss and other “learn to read” books.

The real-time vibroacoustic and electric potential biofield activity captures the temporal dynamics of brain activity during non-verbalized speech production. Participants are asked to think of specific written words and actively say them over and over in their head without vocalization while their “mind vocalization” latencies and vibroacoustic and electric potential biofield activity are recorded. We use group temporal Independent Component Analysis (group tICA) to obtain temporally independent component time courses and their corresponding vibroacoustic and electric potential biofield activity topographic maps in order to quantitatively characterize anatomical sources and their spatio-temporal dynamics.

This is a new method for non-invasively investigating the real-time activity of the human brain. We build here on earlier combined MEG and EEG (EMEG) work which used a novel multivariate pattern analysis method, called spatiotemporal searchlight representational similarity analysis (ssRSA), to decode information about frequency preference and selectivity directly from the dynamic neural activity of the brain as reconstructed in real-time vibroacoustic and electric potential biofield activity source space. This method is an extension of fMRI-based RSA to time-resolved imaging modalities.

The key procedure underpinning ssRSA is the construction of similarity structures that capture the dynamic spatiotemporal patterns of neural activation in EMEG source space. These similarity structures are encoded in a representational dissimilarity matrix (RDM), where each entry in the RDM denotes the computed dissimilarity between the source-space neural responses to pairs of experimental conditions (for example, pairs of different thought words).

In our non-invasive “mind reading” solution, brain data real-time vibroacoustic and electric potential biofield activity RDMs capture the pattern of brain activity at each point of interest in neural space and time, as sampled by ssRSA searchlight parameters. These brain-based similarity/dissimilarity matrices are then related to parallel, theoretically defined similarity structures, known as real-time vibroacoustic and electric potential biofield activity model RDMs for our training set of 400 Dr. Seuss sight word list. Focusing on frequency preferences and selectivity in human auditory processing regions in the temporal cortex, the model real-time vibroacoustic and electric potential biofield activity RDMs encode hypothesized similarities/dissimilarities between sight word resonance frequencies, as derived from a computational model of auditory processing. Critically, the real-time vibroacoustic and electric potential biofield activity ssRSA technique made it possible to relate neural-level patterns of activation directly to abstract functional theories about how auditory cortex is organized.

We structured machine learning sMLssRSA to transparently compute representations of the similarity structure of the brain states generated incrementally as human participants think of unspoken words. These brain data sMLRDMs can then be related to time-varying model sMLRDMs which capture the similarity structure of the machine states extracted during an ASR analysis of the same sets of spoken words.

In certain embodiments, the brain reading system can be trained surreptitiously by observing the environment the subject is in, or responding to, and using the measured responses to train the system. For example, if a subject is passing a billboard and seen looking up at it, their signal output could be assumed to be in response to the words or images on the billboard. Though much more difficult than in a controlled environment, given enough time with a subject, the systems may be trained to an extent that they provide useful data when a subject is subsequently performing subvocalizations.

The ability to understand spoken language is a defining human capacity. But despite decades of research, there is still no well-specified account of how sound entering the ear is neurally interpreted as a sequence of meaningful words. A fundamental concern in the human sciences is to relate the study of the neurobiological systems supporting complex human cognitive functions to the development of computational systems capable of emulating or even surpassing these capacities. Spoken language comprehension is a salient domain that depends on the capacity to recognize fluent speech, decoding word identities and their meanings from a stream of rapidly varying auditory input, and in one example—unspoken, thought speech.

In humans, the language vocalization process is learned subconsciously and very quickly by newborns and depends on a highly dynamic set of electrophysiological processes in speech- and language-related brain areas. These processes extract salient phonetic cues which are mapped onto abstract word identities as a basis for linguistic interpretation. But the exact nature of these processes, their computational content, and the organization of the neural systems that support them, are far from being understood. The rapid, parallel development of Automatic Speech Recognition (ASR) systems, with near-human levels of performance, means that computationally specific solutions to the speech recognition problem are now emerging, built primarily for the goal of optimizing accuracy, with little reference to potential neurobiological constraints and/or physiobiological underpinnings.

With advancements in human-computer interfaces, communication with machines is more intuitive than ever. These natural user interfaces, however, rely on a person's ability to control voluntary movements. What about people who are immobilized or situationally impaired and cannot type, gesticulate, tap, or speak? With these issues in mind, we describe devices, systems, and methods of the present technology that employ novel sensors and data integration methods to provide for non-contact and non-invasive methods of detecting psychological states, intents, and actions, including the detection of unspoken, thought speech, generally referred to herein as “brain reading”.

The US patent application US2006/01293394 describes a subvocalization based computer synthesized speech system for communication. US patent application describes a computer-based shopping assistant employing subvocalization detection. Pasley has described a method of reconstructing speech from the human auditory cortex using spectro-temporal analysis of neurosignals harvested through implantable electrodes in patient undergoing neurosurgical treatment for epilepsy (1a).

Such technology is applicable to many uses, such as, but not limited to facilitating communications in noisy environments, detection of deceptive intent, clandestine operations, brain-machine interfaces and psychotherapy. Unfortunately, existing technology requires the use of invasive sensors in order to achieve useful sensitivity and specificity such as the implantable electrodes used by Pasley. Moreover, because the invasive neural sensors harvest a superposition of electrical fields representing myriad neural functions, it is difficult to disambiguate the signals into intelligible data that accurately represents the desired psychological actions and states.

The devices systems and methods of the current technology provide for non-contact and non-invasive detection and disambiguation of subvocalization and other psychological events and states. Furthermore, the sensors described herein allow for the harvesting of data provides for more sensitive and specific methods of non-contact and non-invasive detection and disambiguation of subvocalization and other psychological events and states than currently existing technologies allow for.

### Airflow Detection

In some embodiments, the vibroacoustic and electrical potential measurements are supplanted or entirely replaced by infrared thermographic imaging of the mouth or nostril regions. We surprisingly found that “mind vocalization” induces subtle air movements through the subject's respiratory system that may be detected by the changing temperature caused by exhaled warm humid air. When this exhalation takes place through the nostrils or mouth, the changes in the thermographic signature around the subject's nostrils or mouth can be readily detected via thermopile sensors. In some embodiments, these measurements can be guided by a separate 3-d imaging system that uses stereoscopic camera arrays, phase detect distance ranging, generally known facial recognition technologies, or through image analysis of images obtained by an array of thermal sensors. The advantage of this method is that it can be deployed surreptitiously and from very far distances such as 1, 2, 3, 4, 5, 10, 25, 50, 100 or more feet. Under certain conditions and using suitable equipment such surveillance may be accomplished from a distance of 100, 200, 300, 400, 500, 1000, 1500, 2500, 5000, or more feet. Under certain weather conditions, where the exhaled air generates condensate as it hits the cold environmental air, the formation of the condensate. In other embodiments, the increased CO₂ or water content of the exhaled air may e detected spectrophotometrically.

For non-surreptitious applications, the airflow, vibroacoustic and electrical potential signals may be detected through sensors placed in earplugs, headsets, headwear, visors, sweatbands, masks, scarfs, eyewear or adhesive patches.

In some embodiments, the devices, systems, and methods of the present technology may be used to help subjects who are paralyzed regain the ability to interact with computers or physical objects.

In some embodiments the devices, systems, and methods of the present technology can be used to interact with social media.

In some embodiments, the devices, systems, and methods of the present technology may be used in music therapy. For example, a system that could analyze a person's emotional state using their neural signals, and then automatically develop an appropriate piece of music. For example, if you're feeling down, the system's algorithms could write you a piece of music to help lift your mood. In other embodiments, the system can drive a speech synthesizer to externally mirror the “mind vocalizations.

In some embodiments, the system can be used to drive a neural stimulation system connected to a second subject allowing direct brain to brain communication. In some embodiments the neural stimulation system is an intracranial magnetic stimulation system.

In another embodiment, the devices, systems, and methods of the present technology can qualify and quantitate emotional states, interpret intent and allow people to control their environment, virtual reality environments, and workplace training, and education using their thoughts. By training workers in a simulated environment and measuring their emotional response, employers can gauge their performance and emotional response, and adapt the training as necessary.

In still other embodiments, the devices, systems, and methods of the present technology reduce or eliminate physical repetitive strain injuries associated with computer-human interface devices.

In other embodiments, the devices, systems, and methods of the present technology are sensitive to the attentional status of a subject and can engage an alarm when a subject becomes inattentive to a particular task, or, overly attentive another task. In other embodiments AR/R may be used to trigger responses or guide the system for testing and/or algorithm training purposes.

People with locked-in syndrome are entirely mentally aware, but can move none, or almost none, of their muscles. They can't speak or write; their ability to communicate with the outside world is limited to perhaps moving an eyelid or a single finger when asked a question. The devices, systems, and methods of the present technology can be employed by those with locked-in syndrome to communicate more fully, being able to use their brain signals to choose letters in order to write messages, send emails and respond to questions.

While useful as brain-machine interfaces, the devices, systems, and methods of the present technology are also useful in the diagnosis and treatment of medical conditions.

### Intracranial Pressure

Serious symptoms that might indicate a life-threatening condition related increased intracranial pressure include: abnormal pupil size or nonreactivity to light, bleeding from the ear after head injury, bruising and swelling around the eyes, change in consciousness, lethargy, or passing out, confusion or disorientation, difficulty breathing or shortness of breath, double vision or other visual symptoms, neurological problems, such as balance issues, numbness and tingling, memory loss, paralysis, slurred or garbled speech, or inability to speak, projectile vomiting, seizure or convulsion, stiff neck, sudden changes or problems with vision, and severe headache.

Symptoms that might indicate a serious or life-threatening condition in infants or toddlers include: abnormal pupil size or nonreactivity to light, bulging of the soft spot on top of the head (fontanel), drowsiness or lethargy, not feeding or responding normally, projectile vomiting.

Increased intracranial pressure is a serious condition in which there is higher than normal pressure inside the skull. Causes include: brain aneurysm rupture (weak area in a brain blood vessel that can rupture and bleed), brain hemorrhage or hematoma (bleeding in the brain due to such causes as head trauma, stroke, or taking “blood thinners”), brain tumor causing pressure within the head, encephalitis (inflammation of the brain commonly due to a viral infection), head injury, hydrocephalus (high levels of fluid in the brain or “water on the brain”), intracranial hypertension (abnormally high pressure of the cerebrospinal fluid in the skull), meningitis (infection or inflammation of the sac around the brain and spinal cord), seizure disorder, and stroke.

Adverse effects of treatments that lower cerebrospinal fluid pressure. Coma, disability and poor quality of life, paralysis, permanent brain damage, including intellectual and cognitive deficits and difficulties moving and speaking, respiratory arrest, seizures and stroke.

### Idiopathic Intracranial Hypertension

Synonyms of Idiopathic Intracranial Hypertension: benign intracranial hypertension and/or pseudotumor cerebri.

In the idiopathic or primary type (IIH), obesity is considered a factor in young women. However, only a small fraction of obese individuals develop IH, so other unknown causes are yet to be determined.

The many potential causes of secondary intracranial hypertension have been noted above. Note that in secondary IH, unlike IIH, obesity, gender, age and race are NOT risk factors, but may be present.

The mechanism by which IH occurs is not known, but several possibilities have been suggested. Most research supports the theory that there is resistance or obstruction to CSF outflow through the normal existing pathways in the brain, leading to relative over-production of CSF.

### Affected Populations

The incidence of IIH in the general population is thought to be about 1 per 100,000. In obese young females the incidence of IIH is about 20 per 100,000. IIH occurs in men and children as well, but with substantially lower frequency. Weight is not usually a factor in men and in children under 10 years of age.

The true incidence of secondary IH remains unknown because of the wide range of underlying causes and the lack of published surveys on the subject. Current statistics are not available on how many people have secondary intracranial hypertension.

### Related Disorders

Symptoms of the following disorders can be similar to those of IIH. Comparisons may be useful for a differential diagnosis:

Arachnoiditis is a progressive inflammatory disorder affecting the middle membrane surrounding the spinal cord and brain (arachnoid membrane). It may affect both the brain and the spinal cord and may be caused by foreign solutions (such as dye) being injected into the spine or arachnoid membrane. Symptoms may include severe headaches, vision disturbances, dizziness, nausea and/or vomiting. If the spine is involved, pain, unusual sensations, weakness and paralysis can develop.

Epiduritis is characterized by inflammation of the tough, outer canvas-like covering surrounding the brain and spinal cord known as the dura mater. Symptoms of this disorder can be similar to IIH.

Meningitis is an inflammation of the membranes around the brain and the spinal cord. It may occur as three different forms; adult, infantile and neonatal. It may also be caused by a number of different infectious agents such as bacteria, viruses, or fungi, or it may be caused by malignant tumors. Meningitis may develop suddenly or have a gradual onset. Symptoms may include fever, headache, a stiff neck, and vomiting. The patient may also be irritable, confused and go from drowsiness, to stupor to coma. (For more information on this disorder, choose “Meningitis” as your search term in the Rare Disease Database.)

Brain tumors may also cause symptoms similar to IIH. Neuroimaging will help with this diagnosis.

### Standard Therapies

Treatment should first and foremost involve lifestyle and dietary modifications in order to promote weight loss for those patients who are overweight or obese. This may even include consultation with a nutritionist or dietician.

Medical treatment consists of using drugs called carbonic anhydrase inhibitors to suppress the production of CSF. The most commonly used of the carbonic anhydrase inhibitors is acetazolamide. A large multicenter, randomized, controlled trial published in 2014 demonstrated that acetazolamide combined with dietary weight loss resulted in improved visual field function, nerve swelling, and quality of life measures, compared to the treatment of dietary changes alone. Carbonic anhydrase inhibitors inhibit the enzyme system needed to produce CSF and control the pressure (by controlling the volume) to some degree. These drugs do not work in all cases and can have potentially serious side effects. Acetazolamide should be avoided in early (1st trimester) pregnancy, and should be used with caution in later stages of pregnancy.

Topiramate is another, second-line, agent sometimes used to treat IH. While it has less potent carbonic anhydrase inhibition, it may be helpful in its capacity as a migraine headache medication. Other potential treatment options include methazolamide and furosemide, however these all of the above agents have not been evaluated as thoroughly as acetazolamide, and further study is needed to establish their utility. Corticosteroids, while used in the past to treat IH, are no longer recommended.

When medical treatment fails and vision is at risk, surgical intervention may be necessary. One of two types of surgery may be performed: optic nerve sheath fenestration, neurosurgical shunt Optic nerve fenestration is a procedure in which a small opening is made in the sheath around the optic nerve in an attempt to relieve swelling (papilledema). Optic nerve sheath fenestration has a high rate of success in protecting vision, but usually does not significantly reduce headaches. Implantation of neurosurgical shunts (internal tubes) is used to drain CSF into other areas of the body. These shunts protect vision and reduce headache, but typically have a higher complication rate than optic nerve sheath fenestration.

### REFERENCES

1a. Pasley B N, David S V, Mesgarani N, Flinker A, Shamma S A, et al. (2012) Reconstructing Speech from Human Auditory Cortex. PLoS Biol 10(1): e1001251. doi:10.1371/journal.pbio.1001251
1b. Matsumae, M. et al. Research into the physiology of cerebrospinal fluid reaches a new horizon: intimate exchange between cerebrospinal fluid and interstitial fluid may contribute to maintenance of homeostasis in the central nervous system. Neurol. medico-chirurgica 56, 416-441 (2016).
2. Eide, P. K. & Sorteberg, W. Diagnostic intracranial pressuremonitoring and surgicalmanagement in idiopathic normal pressure hydrocephalus: A 6-year review of 214 patients. Neurosurg. 66, 80-91 (2010).
3. Jessen, N. A., Munk, A. S. F., Lundgaard, I. & Nedergaard, M. The glymphatic system: a beginner's guide. Neurochem. research 40, 2583-2599 (2015).
4. Czosnyka, M. & Pickard, J. D. Monitoring and interpretation of intracranial pressure. J. Neurol. Neurosurg. & Psychiatry 75, 813-821 (2004).
5. Lindstrom, E. K., Ringstad, G., Mardal, K.-A. & Eide, P. K. Cerebrospinal fluid volumetric net flow rate and direction in idiopathic normal pressure hydrocephalus. NeuroImage: Clin. 20, 731-741 (2018).
6. Bardan, G., Plouraboud, F., Zagzoule, M. & Baledent, O. Simple patient-based transmantle pressure and shear estimate from cine phase-contrast MRI in cerebral aqueduct. IEEE Transactions on Biomed. Eng. 59, 2874-2883 (2012).
7. Ringstad, G. et al. Non-invasive assessment of pulsatile intracranial pressure with phase-contrast magnetic resonance imaging. Plos one 12, e0188896 (2017).
8. Eide, P. K. & Sohle, T. Is ventriculomegaly in idiopathic normal pressure hydrocephalus associated with a transmantle gradient in pulsatile intracranial pressure? Acta neurochirurgica 152, 989-995 (2010).
9. Stephensen, H., Tisell, M. & Wikkelsö, C. There is no transmantle pressure gradient in communicating or noncommunicating hydrocephalus. Neurosurg. 50, 763-773 (2002).
10. Dehaene, S. & Cohen, L. The unique role of the visual word form area in reading. Trends Cogn. Sci. 15, 254-262 (2011).
11. McCandliss, B. D., Cohen, L. & Dehaene, S. The visual word form area: expertise for reading in the fusiform gyrus. Trends Cogn. Sci. 7, 293-299 (2003).
12. Hannagan, T., Amedi, A., Cohen, L., Dehaene-Lambertz, G. & Dehaene, S. Origins of the specialization for letters and numbers in ventral occipitotemporal cortex. Trends Cogn. Sci. 19, 374-382 (2015).
13. Glezer, L. S., Jiang, X. & Riesenhuber, M. Evidence for highly selective neuronal tuning to whole words in the ‘visual word form area’. Neuron 62, 199-204 (2009).
14. Plaut, D. C. & Behrmann, M. Complementary neural representations for faces and words: a computational exploration. Cogn. Neuropsychol. 28, 251-275 (2011).

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

1. A system for monitoring, non-invasively, intracranial pressure of a subject, the system comprising:
a vibroacoustic sensor configured to detect vibroacoustic signals associated with intracranial pressure of the subject, within a bandwidth ranging from about 0.01 Hz to about 20 kHz; and
an electric potential sensor configured to detect electric potential signals reflective of baseline time-based events in the subject for identifying baseline time-based intracranial pressure changes from the detected vibroacoustic signals, wherein the at least one vibroacoustic sensor is housed in a wearable device which is configured to be non-invasively coupled to a head of the subject.

2. The system of claim 1, wherein the system includes a plurality of the vibroacoustic sensors configured to be positioned at different locations on the head of the subject.

3. The system of claim 1, wherein the vibroacoustic sensor comprises at least one voice coil sensor.

4. The system of claim 1, wherein the electric potential sensor is housed in the wearable device.

5. The system of claim 1, wherein the wearable device comprises an earpiece positionable in or over the ear of the subject, and the vibroacoustic sensor comprises a voice coil sensor in the earpiece.

6. The system of claim 5, further comprising a speaker configured to emit a signal, the speaker housed in the earpiece and separated from the voice coil sensor by a dampener.

7. The system of claim 6, wherein the signal is a predetermined vibroacoustic signal pattern retrieved from a sound library.

8. The system of claim 6, wherein the system is configured such that one or both of the vibroacoustic and electric potential sensors measure a respectively one or both of the vibroacoustic and electric potential signals of the subject responsive to the signal being provided to the subject.

9. The system of claim 1, wherein the wearable device comprises two ear pieces, each ear piece positionable in or over a respective ear of the subject, and the vibroacoustic sensor comprises at least one voice coil sensor in each ear piece, whereby the vibroacoustic signals detected in each ear piece can identify differences associated with left and right brain hemispheres of the subject.

10. The system of claim 1, wherein the wearable device comprises two ear pieces, each ear piece positionable in or over a respective ear of the subject, and the vibroacoustic sensor comprises at least one voice coil sensor housed in one ear piece, and a speaker configured to emit a signal housed in the other ear piece.

11. The system of claim 10, wherein the signal is a predetermined vibroacoustic signal pattern retrieved from a sound library, the speaker being configured to emit the predetermined signal pattern.

12. The system of claim 10, wherein the system is configured such that one or both of the vibroacoustic and electric potential sensors measure one or both of the respective vibroacoustic and electric potential signals responsive to the speaker signal provided to the subject.

13. The system of claim 1, wherein the wearable device comprises a patch configured to be non-invasively coupled to a skin of the subject.

14. The system of claim 1, further comprising: a patch configured to be non-invasively coupled to a skin of the subject, the patch including the electric potential sensor or another electric potential sensor.

15. The system of claim 1, further comprising: a patch configured to be non-invasively coupled to a skin of the subject, the patch including another vibroacoustic sensor.

16. The system of claim 1, further comprising: a patch configured to be non-invasively coupled to a skin of the subject, the patch including another vibroacoustic sensor and the electric potential sensor and/or another electric potential sensor.

17. The system of claim 1, further comprising a remote device for providing a signal to the subject, the signal being one or more of a vibroacoustic signal, a sound signal, a haptic signal, and a visual signal.

18. The system of claim 17, wherein the signal is a predetermined vibroacoustic signal pattern retrieved from a sound library, the remote device being configured to emit the predetermined vibroacoustic signal pattern.

19. The system of claim 18, wherein the system is configured such that one or both of the vibroacoustic and electric potential sensors measure one or both of the respective vibroacoustic and electric potential signals from the subject responsive to the signal being provided to the subject by the remote device.

20. The system of claim 17, wherein the remote device includes another electric potential sensor for remotely detecting an electric potential associated with the subject.

21. The system of claim 1, further comprising one or more sensors selected from: an infrared thermographic camera for detecting temperature changes associated with airflow through a nose or a mouth of the subject; a machine vision camera for detecting one or more of: facial movement of the subject, chest movement of the subject, eye tracking of the subject and iris color scanning of the subject; and a sensor for detecting volatile organic compounds emanating from the subject;

22. The system of claim 1, further comprising: an augmented/virtual reality head-piece wearable by the subject.

23. The system of claim 1, wherein the vibroacoustic sensor has a vibroacoustic sensor sampling rate for capturing the vibroacoustic signals and the electric potential sensor has an electric potential sensor sampling rate for capturing the electric potential signals, each of the vibroacoustic sensor sampling rate and the electric potential sensor sampling rate being determined to optimize the battery life of the respective vibroacoustic sensor and the electric potential sensor.

24. The system of claim 1, wherein the vibroacoustic sensor has a vibroacoustic sensor sampling rate for capturing the vibroacoustic signals and the electric potential sensor has an electric potential sensor sampling rate for capturing the electric potential signals, and the respective sampling rates of the vibroacoustic sensor and the electric potential sensor can be switched between a relatively high sampling rate and a relatively low sampling rate to optimize sections of high resolution and optimize battery life respectively.

25-39. (canceled)
